SEQUENCE LISTING (235 sequences)

SEQ ID NO: 1 - DNA, 1219 bp, synthetic construct
  source 1..1219: mol_type = other DNA
gattgcctcagatcacactatctccacttgcccagccctgtggaagattagcggccatgtattccaatgtgataggaactgtaacctctggaaaaaggtacgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagcttgagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggtaccagaggggtgagctttccccttgcctgcccctactgggttttgtgacctccaaaggactcacaggaatgacctccaacacctttgagaagaccaggccctc

SEQ ID NO: 2 - DNA, 25 bp, synthetic construct
  source 1..25: mol_type = other DNA
gattgcctcagatcacactatctcc

SEQ ID NO: 3 - DNA, 21 bp, synthetic construct
  source 1..21: mol_type = other DNA
gagggcctggtcttctcaaag

SEQ ID NO: 4 - DNA, 25 bp, synthetic construct
  source 1..25: mol_type = other DNA
gattgcctcagatcacactatctcc

SEQ ID NO: 5 - DNA, 21 bp, synthetic construct
  source 1..21: mol_type = other DNA
gagggcctggtcttctcaaag

SEQ ID NO: 6 - DNA, 25 bp, synthetic construct
  source 1..25: mol_type = other DNA
gattgcctcagatcacactatctcc

SEQ ID NO: 7 - DNA, 21 bp, synthetic construct
  source 1..21: mol_type = other DNA
gagggcctggtcttctcaaag

SEQ ID NO: 8 - DNA, 25 bp, synthetic construct
  source 1..25: mol_type = other DNA
gattgcctcagatcacactatctcc

SEQ ID NO: 9 - DNA, 21 bp, synthetic construct
  source 1..21: mol_type = other DNA
gagggcctggtcttctcaaag

SEQ ID NO: 10 - DNA, 25 bp, synthetic construct
  source 1..25: mol_type = other DNA
gattgcctcagatcacactatctcc

SEQ ID NO: 11 - DNA, 21 bp, synthetic construct
  source 1..21: mol_type = other DNA
gagggcctggtcttctcaaag

SEQ ID NO: 12 - DNA, 25 bp, synthetic construct
  source 1..25: mol_type = other DNA
gattgcctcagatcacactatctcc

SEQ ID NO: 13 - DNA, 21 bp, synthetic construct
  source 1..21: mol_type = other DNA
gagggcctggtcttctcaaag

SEQ ID NO: 14 - DNA, 25 bp, synthetic construct
  source 1..25: mol_type = other DNA
gattgcctcagatcacactatctcc

SEQ ID NO: 15 - DNA, 21 bp, synthetic construct
  source 1..21: mol_type = other DNA
gagggcctggtcttctcaaag

SEQ ID NO: 16 - DNA, 25 bp, synthetic construct
  source 1..25: mol_type = other DNA
gattgcctcagatcacactatctcc

SEQ ID NO: 17 - DNA, 21 bp, synthetic construct
  source 1..21: mol_type = other DNA
gagggcctggtcttctcaaag

SEQ ID NO: 18 - DNA, 25 bp, synthetic construct
  source 1..25: mol_type = other DNA
gattgcctcagatcacactatctcc

SEQ ID NO: 19 - DNA, 21 bp, synthetic construct
  source 1..21: mol_type = other DNA
gagggcctggtcttctcaaag

SEQ ID NO: 20 - DNA, 25 bp, synthetic construct
  source 1..25: mol_type = other DNA
gattgcctcagatcacactatctcc

SEQ ID NO: 21 - DNA, 21 bp, synthetic construct
  source 1..21: mol_type = other DNA
gagggcctggtcttctcaaag

SEQ ID NO: 22 - DNA, 20 bp, synthetic construct
  source 1..20: mol_type = other DNA
acctctggaaaaaggtaaga

SEQ ID NO: 23 - DNA, 19 bp, synthetic construct
  source 1..19: mol_type = other DNA
agaaccagttttcaggcgg

SEQ ID NO: 24 - DNA, 17 bp, synthetic construct
  source 1..17: mol_type = other DNA
accgcatgtcagcagac

SEQ ID NO: 25 - DNA, 26 bp, synthetic construct
  source 1..26: mol_type = other DNA
tggaaaaaggtacgaattcgagggca

SEQ ID NO: 26 - DNA, 18 bp, synthetic construct
  source 1..18: mol_type = other DNA
cgctaatcttccacaggg

SEQ ID NO: 27 - DNA, 29 bp, synthetic construct
  source 1..29: mol_type = other DNA
tctggaaaaaggtagattgcctcagatca

SEQ ID NO: 28 - DNA, 122 bp, synthetic construct
  source 1..122: mol_type = other DNA
attccaatgtgataggaactgtaacctctggaaaaaggtagaattcttagctctgtttacgtcccagcgggcatgagagtaaagaggggtgagctttccccttgcctgcccctactgggttt

SEQ ID NO: 29 - DNA, 26 bp, synthetic construct
  source 1..26: mol_type = other DNA
attccaatgtgataggaactgtaacc

SEQ ID NO: 30 - DNA, 19 bp, synthetic construct
  source 1..19: mol_type = other DNA
aaacccagtaggggcaggc

SEQ ID NO: 31 - DNA, 26 bp, synthetic construct
  source 1..26: mol_type = other DNA
attccaatgtgataggaactgtaacc

SEQ ID NO: 32 - DNA, 19 bp, synthetic construct
  source 1..19: mol_type = other DNA
aaacccagtaggggcaggc

SEQ ID NO: 33 - DNA, 26 bp, synthetic construct
  source 1..26: mol_type = other DNA
attccaatgtgataggaactgtaacc

SEQ ID NO: 34 - DNA, 19 bp, synthetic construct
  source 1..19: mol_type = other DNA
aaacccagtaggggcaggc

SEQ ID NO: 35 - DNA, 30 bp, synthetic construct
  source 1..30: mol_type = other DNA
attccaatgtgataggaactgtaacctctg

SEQ ID NO: 36 - DNA, 19 bp, synthetic construct
  source 1..19: mol_type = other DNA
aaacccagtaggggcaggc

SEQ ID NO: 37 - DNA, 26 bp, synthetic construct
  source 1..26: mol_type = other DNA
attccaatgtgataggaactgtaacc

SEQ ID NO: 38 - DNA, 19 bp, synthetic construct
  source 1..19: mol_type = other DNA
aaacccagtaggggcaggc

SEQ ID NO: 39 - DNA, 26 bp, synthetic construct
  source 1..26: mol_type = other DNA
attccaatgtgataggaactgtaacc

SEQ ID NO: 40 - DNA, 19 bp, synthetic construct
  source 1..19: mol_type = other DNA
aaacccagtaggggcaggc

SEQ ID NO: 41 - DNA, 30 bp, synthetic construct; identical to SEQ ID NO: 35
  source 1..30: mol_type = other DNA

SEQ ID NO: 42 - DNA, 19 bp, synthetic construct
  source 1..19: mol_type = other DNA
aaacccagtaggggcaggc

SEQ ID NO: 43 - DNA, 30 bp, synthetic construct; identical to SEQ ID NO: 35
  source 1..30: mol_type = other DNA

SEQ ID NO: 44 - DNA, 19 bp, synthetic construct
  source 1..19: mol_type = other DNA
aaacccagtaggggcaggc

SEQ ID NO: 45 - DNA, 22 bp, synthetic construct
  source 1..22: mol_type = other DNA
cttgtccctctttgccttctct

SEQ ID NO: 46 - DNA, 22 bp, synthetic construct
  source 1..22: mol_type = other DNA
gggtggatctgagtggaagaaa

SEQ ID NO: 47 - DNA, 142 bp, synthetic construct
  source 1..142: mol_type = other DNA
gcggccatgtattccaatgtgataggaactgtaacctctggaaaaaggtagaattcttagctctgtttacgtcccagcgggcatgagagtaaagaggggtgagctttccccttgcctgcccctactgggttttgtgacctcc

SEQ ID NO: 48 - DNA, 21 bp, synthetic construct
  source 1..21: mol_type = other DNA
gcggccatgtattccaatgtg

SEQ ID NO: 49 - DNA, 22 bp, synthetic construct
  source 1..22: mol_type = other DNA
ggaggtcacaaaacccagtagg

SEQ ID NO: 50 - DNA, 21 bp, synthetic construct
  source 1..21: mol_type = other DNA
gcggccatgtattccaatgtg

SEQ ID NO: 51 - DNA, 22 bp, synthetic construct
  source 1..22: mol_type = other DNA
ggaggtcacaaaacccagtagg

SEQ ID NO: 52 - DNA, 21 bp, synthetic construct
  source 1..21: mol_type = other DNA
gcggccatgtattccaatgtg

SEQ ID NO: 53 - DNA, 22 bp, synthetic construct
  source 1..22: mol_type = other DNA
ggaggtcacaaaacccagtagg

SEQ ID NO: 54 - DNA, 20 bp, synthetic construct
  source 1..20: mol_type = other DNA
cctctaaggtttgcttacga

SEQ ID NO: 55 - DNA, 20 bp, synthetic construct
  source 1..20: mol_type = other DNA
aattatggggattactagga

SEQ ID NO: 56 - DNA, 20 bp, synthetic construct
  source 1..20: mol_type = other DNA
gccaaggactcaaacccaga

SEQ ID NO: 57 - DNA, 20 bp, synthetic construct
  source 1..20: mol_type = other DNA
ccccgttctcctgtggattc

SEQ ID NO: 58 - DNA, 27 bp, synthetic construct
  source 1..27: mol_type = other DNA
aagaatgttgtgataaaaggtgatgct

SEQ ID NO: 59 - DNA, 24 bp, synthetic construct
  source 1..24: mol_type = other DNA
acacatccatgggacttctgcctc

SEQ ID NO: 60 - DNA, 26 bp, synthetic construct
  source 1..26: mol_type = other DNA
attccaatgtgataggaactgtaacc

SEQ ID NO: 61 - DNA, 19 bp, synthetic construct
  source 1..19: mol_type = other DNA
aaacccagtaggggcaggc

SEQ ID NO: 62 - DNA, 25 bp, synthetic construct
  source 1..25: mol_type = other DNA
ttccaatgtgataggaactgtaacc

SEQ ID NO: 63 - DNA, 18 bp, synthetic construct
  source 1..18: mol_type = other DNA
aacccagtaggggcaggc

SEQ ID NO: 64 - DNA, 26 bp, synthetic construct
  source 1..26: mol_type = other DNA
attccaatgtgataggaactgtaacc

SEQ ID NO: 65 - DNA, 19 bp, synthetic construct
  source 1..19: mol_type = other DNA
aaacccagtaggggcaggc

SEQ ID NO: 66 - DNA, 26 bp, synthetic construct
  source 1..26: mol_type = other DNA
attccaatgtgataggaactgtaacc

SEQ ID NO: 67 - DNA, 19 bp, synthetic construct
  source 1..19: mol_type = other DNA
aaacccagtaggggcaggc

SEQ ID NO: 68 - DNA, 26 bp, synthetic construct
  source 1..26: mol_type = other DNA
attccaatgtgataggaactgtaacc

SEQ ID NO: 69 - DNA, 19 bp, synthetic construct
  source 1..19: mol_type = other DNA
aaacccagtaggggcaggc

SEQ ID NO: 70 - DNA, 27 bp, synthetic construct
  source 1..27: mol_type = other DNA
gattccaatgtgataggaactgtaacc

SEQ ID NO: 71 - DNA, 20 bp, synthetic construct
  source 1..20: mol_type = other DNA
gaaacccagtaggggcaggc

SEQ ID NO: 72 - DNA, 20 bp, synthetic construct
  source 1..20: mol_type = other DNA
tgccctggtaacggccaaag

SEQ ID NO: 73 - DNA, 21 bp, synthetic construct
  source 1..21: mol_type = other DNA
tcggacagaaaggcattcaca

SEQ ID NO: 74 - DNA, 24 bp, synthetic construct
  source 1..24: mol_type = other DNA
caacggcaaagggagaacttaaac

SEQ ID NO: 75 - DNA, 66 bp, synthetic construct
  source 1..66: mol_type = other DNA
gataggaactgtaacctctggaaaaaggtagaattcagaggggtgagctttccccttgcctgcccc

SEQ ID NO: 76 - DNA, 66 bp, synthetic construct
  source 1..66: mol_type = other DNA
ggggcaggcaaggggaaagctcacccctctgaattctacctttttccagaggttacagttcctatc

SEQ ID NO: 77 - DNA, 76 bp, synthetic construct
  source 1..76: mol_type = other DNA
tcgttttcgagataggaactgtaacctctggaaaaaggtagaattcagaggggtgagctttccccttgcctgcccc

SEQ ID NO: 78 - DNA, 76 bp, synthetic construct
  source 1..76: mol_type = other DNA
tcgttttcgaggggcaggcaaggggaaagctcacccctctgaattctacctttttccagaggttacagttcctatc

SEQ ID NO: 79 - DNA, 76 bp, synthetic construct; identical to SEQ ID NO: 77
  source 1..76: mol_type = other DNA

SEQ ID NO: 80 - DNA, 76 bp, synthetic construct; identical to SEQ ID NO: 78
  source 1..76: mol_type = other DNA

SEQ ID NO: 81 - DNA, 67 bp, synthetic construct
  source 1..67: mol_type = other DNA
agataggaactgtaacctctggaaaaaggtagaattcagaggggtgagctttccccttgcctgcccc

SEQ ID NO: 82 - DNA, 67 bp, synthetic construct
  source 1..67: mol_type = other DNA
aggggcaggcaaggggaaagctcacccctctgaattctacctttttccagaggttacagttcctatc

SEQ ID NO: 83 - DNA, 122 bp, synthetic construct
  source 1..122: mol_type = other DNA
agtgccttgtctgtagtgtcaactcattgctgccccttccgaattcttagctctgtttacgtcccagcgggcatgagagtaatagtaatccccataatttagctctccatttcatagtcttt

SEQ ID NO: 84 - DNA, 122 bp, synthetic construct
  source 1..122: mol_type = other DNA
aaggaggaggcctaaggatggggcttttctgtcaccaatcgaattcttagctctgtttacgtcccagcgggcatgagagtaactgtccctagtggccccactgtggggtggaggggacagat

SEQ ID NO: 85 - DNA, 122 bp, synthetic construct
  source 1..122: mol_type = other DNA
tgccaagctctccctcccaggatcctctctggctccatcggaattcttagctctgtttacgtcccagcgggcatgagagtaataagcaaaccttagaggttctggcaaggagagagatggct

SEQ ID NO: 86 - DNA, 122 bp, synthetic construct
  source 1..122: mol_type = other DNA
aggccaatggggaggacatcgatgtcacctccaatgactagaattcttagctctgtttacgtcccagcgggcatgagagtaagggtgggcaaccacaaacccacgagggcagagtgctgctt

SEQ ID NO: 87 - DNA, 21 bp, synthetic construct
  source 1..21: mol_type = other DNA
agtgccttgtctgtagtgtca

SEQ ID NO: 88 - DNA, 30 bp, synthetic construct
  source 1..30: mol_type = other DNA
aaagactatgaaatggagagctaaattatg

SEQ ID NO: 89 - DNA, 21 bp, synthetic construct
  source 1..21: mol_type = other DNA
agtgccttgtctgtagtgtca

SEQ ID NO: 90 - DNA, 30 bp, synthetic construct; identical to SEQ ID NO: 88
  source 1..30: mol_type = other DNA

SEQ ID NO: 91 - DNA, 21 bp, synthetic construct
  source 1..21: mol_type = other DNA
agtgccttgtctgtagtgtca

SEQ ID NO: 92 - DNA, 30 bp, synthetic construct; identical to SEQ ID NO: 88
  source 1..30: mol_type = other DNA

SEQ ID NO: 93 - DNA, 21 bp, synthetic construct
  source 1..21: mol_type = other DNA
agtgccttgtctgtagtgtca

SEQ ID NO: 94 - DNA, 30 bp, synthetic construct; identical to SEQ ID NO: 88
  source 1..30: mol_type = other DNA

SEQ ID NO: 95 - DNA, 21 bp, synthetic construct
  source 1..21: mol_type = other DNA
agtgccttgtctgtagtgtca

SEQ ID NO: 96 - DNA, 30 bp, synthetic construct; identical to SEQ ID NO: 88
  source 1..30: mol_type = other DNA

SEQ ID NO: 97 - DNA, 21 bp, synthetic construct
  source 1..21: mol_type = other DNA
agtgccttgtctgtagtgtca

SEQ ID NO: 98 - DNA, 30 bp, synthetic construct; identical to SEQ ID NO: 88
  source 1..30: mol_type = other DNA

SEQ ID NO: 99 - DNA, 21 bp, synthetic construct
  source 1..21: mol_type = other DNA
aaggaggaggcctaaggatgg

SEQ ID NO: 100 - DNA, 18 bp, synthetic construct
  source 1..18: mol_type = other DNA
atctgtcccctccacccc

SEQ ID NO: 101 - DNA, 21 bp, synthetic construct
  source 1..21: mol_type = other DNA
aaggaggaggcctaaggatgg

SEQ ID NO: 102 - DNA, 18 bp, synthetic construct
  source 1..18: mol_type = other DNA
atctgtcccctccacccc

SEQ ID NO: 103 - DNA, 21 bp, synthetic construct
  source 1..21: mol_type = other DNA
aaggaggaggcctaaggatgg

SEQ ID NO: 104 - DNA, 18 bp, synthetic construct
  source 1..18: mol_type = other DNA
atctgtcccctccacccc

SEQ ID NO: 105 - DNA, 21 bp, synthetic construct
  source 1..21: mol_type = other DNA
aaggaggaggcctaaggatgg

SEQ ID NO: 106 - DNA, 18 bp, synthetic construct
  source 1..18: mol_type = other DNA
atctgtcccctccacccc

SEQ ID NO: 107 - DNA, 21 bp, synthetic construct
  source 1..21: mol_type = other DNA
aaggaggaggcctaaggatgg

SEQ ID NO: 108 - DNA, 18 bp, synthetic construct
  source 1..18: mol_type = other DNA
atctgtcccctccacccc

SEQ ID NO: 109 - DNA, 21 bp, synthetic construct
  source 1..21: mol_type = other DNA
aaggaggaggcctaaggatgg

SEQ ID NO: 110 - DNA, 18 bp, synthetic construct
  source 1..18: mol_type = other DNA
atctgtcccctccacccc

SEQ ID NO: 111 - DNA, 18 bp, synthetic construct
  source 1..18: mol_type = other DNA
tgccaagctctccctccc

SEQ ID NO: 112 - DNA, 21 bp, synthetic construct
  source 1..21: mol_type = other DNA
agccatctctctccttgccag

SEQ ID NO: 113 - DNA, 18 bp, synthetic construct
  source 1..18: mol_type = other DNA
tgccaagctctccctccc

SEQ ID NO: 114 - DNA, 21 bp, synthetic construct
  source 1..21: mol_type = other DNA
agccatctctctccttgccag

SEQ ID NO: 115 - DNA, 18 bp, synthetic construct
  source 1..18: mol_type = other DNA
tgccaagctctccctccc

SEQ ID NO: 116 - DNA, 21 bp, synthetic construct
  source 1..21: mol_type = other DNA
agccatctctctccttgccag

SEQ ID NO: 117 - DNA, 18 bp, synthetic construct
  source 1..18: mol_type = other DNA
tgccaagctctccctccc

SEQ ID NO: 118 - DNA, 21 bp, synthetic construct
  source 1..21: mol_type = other DNA
agccatctctctccttgccag

SEQ ID NO: 119 - DNA, 18 bp, synthetic construct
  source 1..18: mol_type = other DNA
tgccaagctctccctccc

SEQ ID NO: 120 - DNA, 21 bp, synthetic construct
  source 1..21: mol_type = other DNA
agccatctctctccttgccag

SEQ ID NO: 121 - DNA, 18 bp, synthetic construct
  source 1..18: mol_type = other DNA
tgccaagctctccctccc

SEQ ID NO: 122 - DNA, 21 bp, synthetic construct
  source 1..21: mol_type = other DNA
agccatctctctccttgccag

SEQ ID NO: 123 - DNA, 20 bp, synthetic construct
  source 1..20: mol_type = other DNA
aggccaatggggaggacatc

SEQ ID NO: 124 - DNA, 19 bp, synthetic construct
  source 1..19: mol_type = other DNA
aagcagcactctgccctcg

SEQ ID NO: 125 - DNA, 20 bp, synthetic construct
  source 1..20: mol_type = other DNA
aggccaatggggaggacatc

SEQ ID NO: 126 - DNA, 19 bp, synthetic construct
  source 1..19: mol_type = other DNA
aagcagcactctgccctcg

SEQ ID NO: 127 - DNA, 20 bp, synthetic construct
  source 1..20: mol_type = other DNA
aggccaatggggaggacatc

SEQ ID NO: 128 - DNA, 19 bp, synthetic construct
  source 1..19: mol_type = other DNA
aagcagcactctgccctcg

SEQ ID NO: 129 - DNA, 20 bp, synthetic construct
  source 1..20: mol_type = other DNA
aggccaatggggaggacatc

SEQ ID NO: 130 - DNA, 19 bp, synthetic construct
  source 1..19: mol_type = other DNA
aagcagcactctgccctcg

SEQ ID NO: 131 - DNA, 20 bp, synthetic construct
  source 1..20: mol_type = other DNA
aggccaatggggaggacatc

SEQ ID NO: 132 - DNA, 19 bp, synthetic construct
  source 1..19: mol_type = other DNA
aagcagcactctgccctcg

SEQ ID NO: 133 - DNA, 20 bp, synthetic construct
  source 1..20: mol_type = other DNA
aggccaatggggaggacatc

SEQ ID NO: 134 - DNA, 19 bp, synthetic construct
  source 1..19: mol_type = other DNA
aagcagcactctgccctcg

SEQ ID NO: 135 - DNA, 20 bp, synthetic construct
  source 1..20: mol_type = other DNA
aattatggggattactagga

SEQ ID NO: 136 - DNA, 20 bp, synthetic construct
  source 1..20: mol_type = other DNA
ggggccactagggacaggat

SEQ ID NO: 137 - DNA, 20 bp, synthetic construct
  source 1..20: mol_type = other DNA
cctctaaggtttgcttacga

SEQ ID NO: 138 - DNA, 20 bp, synthetic construct
  source 1..20: mol_type = other DNA
gtcacctccaatgactaggg

SEQ ID NO: 139 - DNA, 53 bp, synthetic construct
  source 1..53: mol_type = other DNA
acactctttccctacacgacgctcttccgatctcagaactgtccttcaggttc

SEQ ID NO: 140 - DNA, 55 bp, synthetic construct
  source 1..55: mol_type = other DNA
gtgactggagttcagacgtgtgctcttccgatctcactgtttcatttcatccgtg

SEQ ID NO: 141 - DNA, 54 bp, synthetic construct
  source 1..54: mol_type = other DNA
acactctttccctacacgacgctcttccgatctgagagatggctccaggaaatg

SEQ ID NO: 142 - DNA, 55 bp, synthetic construct
  source 1..55: mol_type = other DNA
gtgactggagttcagacgtgtgctcttccgatctcacttcaggacagcatgtttg

SEQ ID NO: 143 - DNA, 53 bp, synthetic construct
  source 1..53: mol_type = other DNA
acactctttccctacacgacgctcttccgatctgatcagtgaaacgcaccaga

SEQ ID NO: 144 - DNA, 58 bp, synthetic construct
  source 1..58: mol_type = other DNA
gtgactggagttcagacgtgtgctcttccgatctcctccttcctagtctcctgatatt

SEQ ID NO: 145 - DNA, 52 bp, synthetic construct
  source 1..52: mol_type = other DNA
acactctttccctacacgacgctcttccgatctagaagaagaagggctccca

SEQ ID NO: 146 - DNA, 51 bp, synthetic construct
  source 1..51: mol_type = other DNA
gtgactggagttcagacgtgtgctcttccgatctcagggagtggccagagt

SEQ ID NO: 147 - DNA, 502 bp, synthetic construct
  source 1..502: mol_type = other DNA
gattgcctcagatcacactatctccacttgcccagccctgtggaagattagcggccatgtattccaatgtgataggaactgtaacctctggaaaaaggtacgaattcgagggcagaggcagtctgctgacatgcggtgacgtggaagagaatcccggcccttctagataactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggtaccagaggggtgagctttccccttgcctgcccctactgggttttgtgacctccaaaggactcacaggaatgacctccaacacctttgagaagaccaggccctc

SEQ ID NO: 148 - DNA, 700 bp, synthetic construct
  source 1..700: mol_type = other DNA
gattgcctcagatcacactatctccacttgcccagccctgtggaagattagcggccatgtattccaatgtgataggaactgtaacctctggaaaaaggtacgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggtaccagaggggtgagctttccccttgcctgcccctactgggttttgtgacctccaaaggactcacaggaatgacctccaacacctttgagaagaccaggccctc

SEQ ID NO: 149 - DNA, 1219 bp, synthetic construct; identical to SEQ ID NO: 1
  source 1..1219: mol_type = other DNA

SEQ ID NO: 150 - DNA, 502 bp, synthetic construct
  source 1..502: mol_type = other DNA
ctccctccctggcccaggtgaaggtgtggttccagaaccggaggacaaagtacaaacggcagaagctggaggaggaagggcctgagtccgagcagaagaacgaattcgagggcagaggcagtctgctgacatgcggtgacgtggaagagaatcccggcccttctagataactgtgccttctagttgccagccatctgttgtttgcgcctcactcgtgccttcattgaccctggaaggtgccactcccactgtcctttcctaataaaatgaggaaattgcatcgcattgtctgagtaggtgtcattctattctggcgtatcgagtggctcaggacagcaagagcgaggattgggaagacaatagcaggcatgctggggatgcggtgggctctatggcggtaccgaagggctcccatcacatcaaccggtggcgcattgccacgaagcaggccaatggggaggacatcgatgtcacctccaatgactagggtgggcaaccacaa

SEQ ID NO: 151 - DNA, 700 bp, synthetic construct
  source 1..700: mol_type = other DNA
ctccctccctggcccaggtgaaggtgtggttccagaaccggaggacaaagtacaaacggcagaagctggaggaggaagggcctgagtccgagcagaagaacgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggagagggcgatgccacctacggcaagctgaccctgaagttcatctgcaccaccggcaagctgccagtgccctggcctaccctcgtgaccaccctgacctaactgtgccttctagttgccagccatctgttgtttgcgcctcactcgtgccttcattgaccctggaaggtgccactcccactgtcctttcctaataaaatgaggaaattgcatcgcattgtctgagtaggtgtcattctattctggcgtatcgagtggctcaggacagcaagagcgaggattgggaagacaatagcaggcatgctggggatgcggtgggctctatggcggtaccgaagggctcccatcacatcaaccggtggcgcattgccacgaagcaggccaatggggaggacatcgatgtcacctccaatgactagggtgggcaaccacaa

SEQ ID NO: 152 - DNA, 1219 bp, synthetic construct
  source 1..1219: mol_type = other DNA
ctccctccctggcccaggtgaaggtgtggttccagaaccggaggacaaagtacaaacggcagaagctggaggaggaagggcctgagtccgagcagaagaacgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggagagggcgatgccacctacggcaagctgaccctgaagttcatctgcaccaccggcaagctgccagtgccctggcctaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgtgccgaggtgaagttcgaaggcgacaccctggtgaaccgcatcgagctgaagggcatcgacttcaaggaggacggcaacatcctggggcacaagcttgagtacaactacaacagccacaacgtctatatcatggccgacaagcagaagaacggcatcaaggtgaacttcaagatccgccacaacatcgaggacggtagcgtgcagctcgctgaccactaccagcagaacactcctatcggagacggtcctgtgctgctgccagacaaccactacctgagcacacagtccgctctgagcaaagaccctaacgagaagcgcgatcacatggtcctgctggagttcgtgacagccgctgggatcactctcggcatggacgagctgtacaagtaactgtgccttctagttgccagccatctgttgtttgcgcctcactcgtgccttcattgaccctggaaggtgccactcccactgtcctttcctaataaaatgaggaaattgcatcgcattgtctgagtaggtgtcattctattctggcgtatcgagtggctcaggacagcaagagcgaggattgggaagacaatagcaggcatgctggggatgcggtgggctctatggcggtaccgaagggctcccatcacatcaaccggtggcgcattgccacgaagcaggccaatggggaggacatcgatgtcacctccaatgactagggtgggcaaccacaa

SEQ ID NO: 153 - DNA, 32 bp, synthetic construct
  source 1..32: mol_type = other DNA
gattgcctcagatcacactatctccacttgcc

SEQ ID NO: 154 - DNA, 27 bp, synthetic construct
  source 1..27: mol_type = other DNA
gagggcctggtcttctcaaaggtgttg

SEQ ID NO: 155 - DNA, 32 bp, synthetic construct; identical to SEQ ID NO: 153
  source 1..32: mol_type = other DNA

SEQ ID NO: 156 - DNA, 27 bp, synthetic construct
  source 1..27: mol_type = other DNA
gagggcctggtcttctcaaaggtgttg

SEQ ID NO: 157 - DNA, 23 bp, synthetic construct
  source 1..23: mol_type = other DNA
ctccctccctggcccaggtgaag

SEQ ID NO: 158 - DNA, 27 bp, synthetic construct
  source 1..27: mol_type = other DNA
ttgtggttgcccaccctagtcattgga

SEQ ID NO: 159 - DNA, 23 bp, synthetic construct
  source 1..23: mol_type = other DNA
ctccctccctggcccaggtgaag

SEQ ID NO: 160 - DNA, 27 bp, synthetic construct
  source 1..27: mol_type = other DNA
ttgtggttgcccaccctagtcattgga

SEQ ID NO: 161 - DNA, 20 bp, synthetic construct
  source 1..20: mol_type = other DNA
gagtccgagcagaagaagaa

SEQ ID NO: 162 - DNA, 47 bp, synthetic construct
  source 1..47: mol_type = other DNA
tttctgttggtgctgatattgcctttatgtgattgctgtatgtctcc

SEQ ID NO: 163 - DNA, 42 bp, synthetic construct
  source 1..42: mol_type = other DNA
acttgcctgtcgctctatcttcgaatctgccaggtgctgata

SEQ ID NO: 164 - DNA, 43 bp, synthetic construct
  source 1..43: mol_type = other DNA
tttctgttggtgctgatattgcctgtgctttacccagttctct

SEQ ID NO: 165 - DNA, 42 bp, synthetic construct
  source 1..42: mol_type = other DNA
acttgcctgtcgctctatcttcgctgggtctctgacatcttt

SEQ ID NO: 166 - DNA, 32 bp, synthetic construct; identical to SEQ ID NO: 153
  source 1..32: mol_type = other DNA

SEQ ID NO: 167 - DNA, 27 bp, synthetic construct
  source 1..27: mol_type = other DNA
gagggcctggtcttctcaaaggtgttg

SEQ ID NO: 168 - DNA, 32 bp, synthetic construct; identical to SEQ ID NO: 153
  source 1..32: mol_type = other DNA

SEQ ID NO: 169 - DNA, 27 bp, synthetic construct
  source 1..27: mol_type = other DNA
gagggcctggtcttctcaaaggtgttg

SEQ ID NO: 170 - DNA, 23 bp, synthetic construct
  source 1..23: mol_type = other DNA
ctccctccctggcccaggtgaag

SEQ ID NO: 171 - DNA, 27 bp, synthetic construct
  source 1..27: mol_type = other DNA
ttgtggttgcccaccctagtcattgga

SEQ ID NO: 172 - DNA, 23 bp, synthetic construct
  source 1..23: mol_type = other DNA
ctccctccctggcccaggtgaag

SEQ ID NO: 173 - DNA, 26 bp, synthetic construct
  source 1..26: mol_type = other DNA
ttgtggttgcccaccctagtcattgg

SEQ ID NO: 174 - DNA, 30 bp, synthetic construct
  source 1..30: mol_type = other DNA
gtcgtaccgactggtagatgacagcaaacc

SEQ ID NO: 175 - DNA, 29 bp, synthetic construct
  source 1..29: mol_type = other DNA
ggtctcgactatacgcccgttttcggatc

SEQ ID NO: 176 - DNA, 30 bp, synthetic construct; identical to SEQ ID NO: 174
  source 1..30: mol_type = other DNA

SEQ ID NO: 177 - DNA, 29 bp, synthetic construct
  source 1..29: mol_type = other DNA
ggtctcgactatacgcccgttttcggatc

SEQ ID NO: 178 - DNA, 30 bp, synthetic construct; identical to SEQ ID NO: 174
  source 1..30: mol_type = other DNA

SEQ ID NO: 179 - DNA, 29 bp, synthetic construct
  source 1..29: mol_type = other DNA
ggtctcgactatacgcccgttttcggatc

SEQ ID NO: 180 - DNA, 30 bp, synthetic construct; identical to SEQ ID NO: 174
  source 1..30: mol_type = other DNA

SEQ ID NO: 181 - DNA, 29 bp, synthetic construct
  source 1..29: mol_type = other DNA
ggtctcgactatacgcccgttttcggatc

SEQ ID NO: 182 - DNA, 1180 bp, synthetic construct
  source 1..1180: mol_type = other DNA
aacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaggagggatctggcgccaccaatttcagcctgctgaaacaggctggcgacgtggaagagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctca

SEQ ID NO: 183 - DNA, 1188 bp, synthetic construct
  source 1..1188: mol_type = other DNA
gtcgtaccgactggtagatgacagcaaacctgttcccttttcggctctgcaacaccgcctagaccgaccggatacacgggtagggcttccgctttacccgtctccctcctggcgcttgtcctcctctcccagtcggcaccacagcggtggctgccgggcgtggtgtcggtgggtcggttggtttttgtctcaccgttggtgtccgtgccgttcagttgcccgccatggctggatctggtggtactagtggaagcaagggtgaggagctgttcaccggagtggtgcctatcctggtcgagctggacggcgacgtaaacggtcacaagttcagcgtgcgtggtgagggcgagggcgatgccaccaacggcaagctgaccctgaagttcatctgcaccactggcaagctgcctgttccatggccaaccctcgtgactacactgacctacggcgttcagtgcttcagccgttaccctgaccatatgaagcgtcacgacttcttcaagtctgccatgcctgaaggctacgtccaggagcgtaccatcagcttcaaggacgatggcacctacaagactcgtgccgaggtgaagttcgagggtgacaccctggtgaaccgcatcgagctgaagggtatcgacttcaaggaggacggcaacatcctgggtcacaagctggagtacaacttcaacagccacaacgtctatatcaccgccgacaagcagaagaacggcatcaaggccaacttcaagattcgtcacaacgtggaggacggtagcgtgcagctcgcagaccactaccagcagaacacgcctatcggcgacggtccagtgttgctgccagacaaccactacctgagcacccagtccgtgctgagcaaagacccgaacgagaagcgcgatcacatggtcctgctggagttcgtgaccgcagccggtatcactggaaccggtgctggaagtggtgagctggatccgttcggcgcccctgccggcgcccctggcggtcccgcgctggggaacggagtggccggcgccggcgaagaagacccggctgcggccttcttggcgcagcaagagagcgagattgcgggcatcgagaacgacgaggccttcgccatcctggacggcggcgcccccgggccccagccgcacggcgagccgccgatccgaaaacgggcgtatagtcgagacc

SEQ ID NO: 184 - DNA, 1129 bp, synthetic construct
  source 1..1129: mol_type = other DNA
tcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcggatcgggtgggactagtggcagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctca

SEQ ID NO: 185 - DNA, 20 bp, synthetic construct
  source 1..20: mol_type = other DNA
cctccaaggagtaagacccc

SEQ ID NO: 186 - DNA, 20 bp, synthetic construct
  source 1..20: mol_type = other DNA
gaacggatccagctcagcca

SEQ ID NO: 187 - DNA, 20 bp, synthetic construct
  source 1..20: mol_type = other DNA
ggtagtcgtactcgtcgtcg

SEQ ID NO: 188 - DNA, 34 bp, synthetic construct
  source 1..34: mol_type = other DNA
acgaagtgttggatataagccagactgtaagtga

SEQ ID NO: 189 - DNA, 46 bp, synthetic construct
  source 1..46: mol_type = other DNA
tctaagcaattataagccatttcacataaaactcttttaggttaaa

SEQ ID NO: 190 - DNA, 34 bp, synthetic construct; identical to SEQ ID NO: 188
  source 1..34: mol_type = other DNA

SEQ ID NO: 191 - DNA, 46 bp, synthetic construct; identical to SEQ ID NO: 189
  source 1..46: mol_type = other DNA

SEQ ID NO: 192 - DNA, 41 bp, synthetic construct
  source 1..41: mol_type = other DNA
gccctgtagtctctctgtatgttatatgtcacattttgtaa

SEQ ID NO: 193 - DNA, 40 bp, synthetic construct
  source 1..40: mol_type = other DNA
aagtaattcacttacagtctggcttatatccaacacttcg

SEQ ID NO: 194 - DNA, 41 bp, synthetic construct; identical to SEQ ID NO: 192
  source 1..41: mol_type = other DNA

SEQ ID NO: 195 - DNA, 40 bp, synthetic construct; identical to SEQ ID NO: 193
  source 1..40: mol_type = other DNA

SEQ ID NO: 196 - DNA, 24 bp, synthetic construct
  source 1..24: mol_type = other DNA
agcttgctggtgaaaaggacccca

SEQ ID NO: 197 - DNA, 46 bp, synthetic construct
  source 1..46: mol_type = other DNA
aatgtgcctctctacaaatattctctaagcaattataagccatttc

SEQ ID NO: 198 - DNA, 24 bp, synthetic construct
  source 1..24: mol_type = other DNA
agcttgctggtgaaaaggacccca

SEQ ID NO: 199 - DNA, 46 bp, synthetic construct; identical to SEQ ID NO: 197
  source 1..46: mol_type = other DNA

SEQ ID NO: 200 - DNA, 34 bp, synthetic construct
  source 1..34: mol_type = other DNA
acgtcagtcttctcttttgtaatgccctgtagtc

SEQ ID NO: 201 - DNA, 45 bp, synthetic construct
  source 1..45: mol_type = other DNA
gatggttaaatgattgacaaaaaaagtaattcacttacagtctgg

SEQ ID NO: 202 - DNA, 34 bp, synthetic construct; identical to SEQ ID NO: 200
  source 1..34: mol_type = other DNA

SEQ ID NO: 203 - DNA, 45 bp, synthetic construct; identical to SEQ ID NO: 201
  source 1..45: mol_type = other DNA

SEQ ID NO: 204 - DNA, 46 bp, synthetic construct
  source 1..46: mol_type = other DNA
tgtagtctctctgtatgttatatgtcacattttgtaattaacagct

SEQ ID NO: 205 - DNA, 46 bp, synthetic construct
  source 1..46: mol_type = other DNA
atttagataaagaaaacatcacttttaaatctaatactggcaaatg

SEQ ID NO: 206 - DNA, 46 bp, synthetic construct; identical to SEQ ID NO: 204
  source 1..46: mol_type = other DNA

SEQ ID NO: 207 - DNA, 47 bp, synthetic construct
  source 1..47: mol_type = other DNA
matttagataaagaaaacatcacttttaaatctaatactggcaaatg

SEQ ID NO: 208 - DNA, 33 bp, synthetic construct
  source 1..33: mol_type = other DNA
catggtacactcagcacggatgaaatgaaacag

SEQ ID NO: 209 - DNA, 46 bp, synthetic construct
  source 1..46: mol_type = other DNA
agcaattataagccatttcacataaaactcttttaggttaaagatg

SEQ ID NO: 210 - DNA, 33 bp, synthetic construct; identical to SEQ ID NO: 208
  source 1..33: mol_type = other DNA

SEQ ID NO: 211 - DNA, 46 bp, synthetic construct; identical to SEQ ID NO: 209
  source 1..46: mol_type = other DNA

SEQ ID NO: 212 - DNA, 42 bp, synthetic construct
  source 1..42: mol_type = other DNA
tctcagattccagtttcagcaaatttgcttgatatgtacagc

SEQ ID NO: 213 - DNA, 36 bp, synthetic construct
  source 1..36: mol_type = other DNA
tgaatagagtggttgcacaaacttacggatcatttg

SEQ ID NO: 214 - DNA, 23 bp, synthetic construct
  source 1..23: mol_type = other DNA
atggtgagcaagggcgaggagct

SEQ ID NO: 215 - DNA, 22 bp, synthetic construct
  source 1..22: mol_type = other DNA
agagtgatcccggcggcggtca

SEQ ID NO: 216 - DNA, 28 bp, synthetic construct
  source 1..28: mol_type = other DNA
cccacaattcgctctcaccaaacctgag

SEQ ID NO: 217 - DNA, 61 bp, synthetic construct
  source 1..61: mol_type = other DNA
agtagtaatagtagtagtattaaataatttgataaataattttagcaatatagttttttgt

SEQ ID NO: 218 - DNA, 28 bp, synthetic construct
  source 1..28: mol_type = other DNA
cccacaattcgctctcaccaaacctgag

SEQ ID NO: 219 - DNA, 61 bp, synthetic construct; identical to SEQ ID NO: 217
  source 1..61: mol_type = other DNA

SEQ ID NO: 220 - DNA, 28 bp, synthetic construct
  source 1..28: mol_type = other DNA
cccacaattcgctctcaccaaacctgag

SEQ ID NO: 221 - DNA, 61 bp, synthetic construct; identical to SEQ ID NO: 217
  source 1..61: mol_type = other DNA

SEQ ID NO: 222 - DNA, 28 bp, synthetic construct
  source 1..28: mol_type = other DNA
cccacaattcgctctcaccaaacctgag

SEQ ID NO: 223 - DNA, 61 bp, synthetic construct; identical to SEQ ID NO: 217
  source 1..61: mol_type = other DNA

SEQ ID NO: 224 - DNA, 39 bp, synthetic construct
  source 1..39: mol_type = other DNA
ggtacaagtggatttgactaattacgagtggcttgataa

SEQ ID NO: 225 - DNA, 39 bp, synthetic construct
  source 1..39: mol_type = other DNA
aaacaatgcactcacttcttcctagagaagagtacattc

SEQ ID NO: 226 - DNA, 49 bp, synthetic construct
  source 1..49: mol_type = other DNA
cctattaaataaaagaataagcagtattattaagtagccctgcatttca

SEQ ID NO: 227 - DNA, 34 bp, synthetic construct
  source 1..34: mol_type = other DNA
catctgcttttttcccgtgtcattctctggactg

SEQ ID NO: 228 - DNA, 28 bp, synthetic construct
  source 1..28: mol_type = other DNA
cccacaattcgctctcaccaaacctgag

SEQ ID NO: 229 - DNA, 61 bp, synthetic construct; identical to SEQ ID NO: 217
  source 1..61: mol_type = other DNA

SEQ ID NO: 230 - DNA, 28 bp, synthetic construct
  source 1..28: mol_type = other DNA
cccacaattcgctctcaccaaacctgag

SEQ ID NO: 231 - DNA, 61 bp, synthetic construct; identical to SEQ ID NO: 217
  source 1..61: mol_type = other DNA

SEQ ID NO: 232 - DNA, 28 bp, synthetic construct
  source 1..28: mol_type = other DNA
cccacaattcgctctcaccaaacctgag

SEQ ID NO: 233 - DNA, 61 bp, synthetic construct; identical to SEQ ID NO: 217
  source 1..61: mol_type = other DNA

SEQ ID NO: 234 - DNA, 27 bp, synthetic construct
  source 1..27: mol_type = other DNA
ccacaattcgctctcaccaaacctgag

SEQ ID NO: 235 - DNA, 61 bp, synthetic construct; identical to SEQ ID NO: 217
  source 1..61: mol_type = other DNA

(19) United States
(12) Patent Application Publication
(10) Pub. No.: US 2024/0309350 A1
(43) Pub. Date: Sep. 19, 2024

(54) MODIFIED DOUBLE-STRANDED DONOR TEMPLATES

(71) Applicant: INTEGRATED DNA TECHNOLOGIES, INC., Coralville, IA (US)

(72) Inventors: Jessica Woodley, North Liberty, IA (US); Bernice Thommandru, Iowa City, IA (US); Joseph Dobosy, Coralville, IA (US); Mark Behlke, Coralville, IA (US); Adam Clore, Iowa City, IA (US); Garrett Rettig, Coralville, IA (US); Beimeng Sun, North Liberty, IA (US)

(21) Appl. No.: 18/679,324

(22) Filed: May 30, 2024

Related U.S. Application Data
(62) Division of application No. 17/079,097, filed on Oct. 23, 2020.
(60) Provisional application No. 62/925,366, filed on Oct. 24, 2019.

(51) Int. Cl. C12N 9/22; C12N 15/113; C12N 15/66

(52) U.S. Cl. CPC C12N 9/22; C12N 15/113; C12N 15/66; C12N 2310/122; C12N 2310/20; C12N 2310/321; C12N 2310/531; C12N 2800/80

(57) ABSTRACT

Described herein are compositions and methods for improving homology directed repair (HDR) efficiency and reducing homology-independent integration following introduction of double strand breaks with engineered nucleases. Additionally, modifications to double stranded DNA donors to improve the donor potency and efficiency of homology directed repair following introduction of double stranded breaks with programmable nucleases.

### CROSS-REFERENCE TO RELATED APPLICATIONS

This application is a divisional of U.S. patent application Ser. No. 17/079,097, filed Oct. 23, 2020, which claims priority to U.S. Provisional Patent Application No. 62/925,366, filed on Oct. 24, 2019, each of which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “013670-9060-US03_sequence_listing_xml_4-JUN-2024,” was created on Jun. 4, 2024, contains 235 sequences, has a file size of 216 kilobytes (221,184 bytes) and is incorporated by reference in its entirety into the specification.

### TECHNICAL FIELD

Described herein are compositions and methods for improving homology directed repair (HDR) efficiency and reducing homology-independent integration following introduction of double strand breaks with engineered nucleases. Additionally, modifications to double stranded DNA donors to improve the donor potency and efficiency of homology directed repair following introduction of double stranded breaks with programmable nucleases.

### BACKGROUND

Genome editing with programmable nucleases allows the site-specific introduction of DNA into target genomes of interest. A number of systems permit targeted genomic editing and these systems include transcription activator-like effector nucleases (TALENs), zinc fingers (ZFNs), or clustered, regularly interspaced, short palindromic repeat (CRISPR).

The CRISPR-Cas9 system has been widely utilized to perform site-specific genome editing in eukaryotic cells. A sequence specific guide RNA is required to recruit Cas9 protein to the target site, and then the Cas9 endonuclease cleaves both strands of the target DNA creating a double stranded break (DSB). This DSB is corrected by the cell's innate DNA damage repair pathways. Two of the main pathways of DSB repair are the error prone non-homologous end joining (NHEJ) pathway, which can lead to random insertions or deletions (indels) in the target DNA, and the homology directed repair (HDR) pathway, which uses a single or double stranded DNA molecule with homology to either side of the DSB as a repair template to generate a desired mutation in the target DNA [1].

Various forms of DNA can be used as the repair template for HDR experiments such as plasmid DNA, double stranded linear DNA (dsDNA), or single stranded DNA (ssDNA). Both dsDNA and ssDNA donors can induce an innate immune response in mammalian tissue culture cells. For short insertions (generally ≤120 bp) or mutations, a chemically synthesized oligonucleotide such as an IDT® Ultramer™ ssDNA can be used as the single stranded oligo donors (ssODN) for HDR experiments. The use of synthetic ssDNA allows for chemical modifications to be placed in the molecule to potentially improve HDR efficiency. Templates for larger insertions (generally >120 bp) are limited due to the increased complexity of synthesis. Generation of long ssDNA can be a labor intensive and costly process, while linear dsDNA can be generated quickly and in large quantities. Because, the more prevalent NHEJ repair pathway facilitates the ligation of blunt ends, a linear dsDNA donor has a higher risk for homology-independent integration into any DSB present in the cell (including the on-target Cas9 cleavage site, any Cas9 off-target sites, and any endogenous DSB) [2, 3]. When homology-independent integration occurs at the on-target site, the entire donor is incorporated including the homology arms leading to the duplication of one or both homology arm regions.

It has been reported that the addition of a 5′-biotin modification on a linear dsDNA donor can reduce the formation of concatemers and integration via the NHEJ pathway [4]. Similarly, another group reported that biotin or ssDNA overhangs on the 5′-terminus can reduce blunt insertions [5]. Another group suggested that TEG and 2′-OMe ribonucleotide adapters on the 5′-termini of dsDNA donors could potentially increase HDR rates by limiting access of the NHEJ machinery to the free ends of the donor but did not demonstrate any reduction in blunt integration [6].

There is a need for compositions of modified dsDNA templates for HDR and methods thereof that increase the efficiency of HDR and reduce undesired homology-independent integration (both at the targeted site and potential off-target or endogenous DSBs) that is typically associated with linear dsDNA donors.

### SUMMARY

One embodiment described herein is a double stranded DNA homology directed repair (HDR) donor comprising: a first homology arm region, an insert region, and a second homology arm region; wherein the first homology arm region and the second homology arm region comprise modifications to one or more nucleotides at or near the 5′-termini. In one aspect, the modifications comprise: modifications to the 2′-position of one or more nucleotides at or near the 5′-terminus of the first homology arm region and modifications to the 2′-position of one or more nucleotides at or near the 5′-terminus of the second homology arm region. In another aspect, the modifications comprise modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region. In another aspect, the modifications at or near the 5′-termini of the double stranded DNA HDR donor comprise one or more of: 2′-O-methyl (2′-OMe), 2′-fluoro (2′-F), or 2′-O-methoxylethyl (2′-MOE). In another aspect, the modifications at or near the 5′-termini of the double stranded DNA HDR donor comprise 2′-MOE. In another aspect, the modification at or near the 5′-termini are non-template mismatches relative to a target DNA. In another aspect, the first homology arm region and the second homology arm region are 40 to 150 nucleotides in length. In another aspect, the first homology arm region and the second homology arm region are at least 100 nucleotides in length. In another aspect, the double stranded DNA HDR donor further comprises universal primer sequences. In another aspect, the insert region is greater than 100 bp. In aspect, the insert region is greater than 0.25 kb, greater than 0.5 kb, greater than 1 kb, greater than 2 kb, greater than 3 kb, greater 4 kb, greater than 5 kb, greater than 6kb, greater than 7 kb, greater than 8 kb, greater than 9 kb, or greater than 10 kb. In another aspect, the double stranded HDR donor comprises a hairpin at either the 5′-terminus or the 3′-terminus. In another aspect, the double stranded HDR donor comprises a hairpin at both the 5′-terminus and the 3′-terminus. In another aspect, the double stranded DNA HDR donor improves homology directed repair efficiency and reduces homology-independent integration in a programmable nuclease system.

Another embodiment described herein is a programmable nuclease system comprising: a modified double stranded DNA homology directed repair (HDR) donor, a programmable nuclease enzyme, and a gRNA, wherein the gRNA molecule is capable of targeting the programmable nuclease molecule to a target nucleic acid. In one aspect, the modified double stranded DNA HDR donor comprises a first homology arm region, an insert region, and a second homology arm region; wherein the first homology arm region and the second homology arm region comprises modifications to one or more nucleotides at or near the 5′-termini. In another aspect, the modified double stranded DNA HDR donor comprises modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region. In another aspect, the modified double stranded DNA HDR donor comprises at least one 2′-OME, 2′-F, or 2′-MOE modifications one or more nucleotides at or near the 5′-termini. In another aspect, the modified double stranded DNA HDR donor comprises one or more 2′-MOE modifications at or near the 5′-termini. In another aspect, the modified double stranded DNA HDR donor comprises universal primer sequences. In another aspect, the modified double stranded DNA HDR donor improves homology directed repair efficiency and reduces homology-independent integration in a programmable nuclease system. In another aspect, the programmable nuclease system comprises one or more of transcription activator-like effector nucleases (TALENs), zinc fingers (ZFNs), or clustered, regularly interspaced, short palindromic repeat (CRISPR). In another aspect, the programmable nuclease system is CRISPR. In another aspect, the programmable nuclease enzyme is CRISPR associated-9 (Cas9). In another aspect, the programmable nuclease system further comprises one or more HDR enhancers.

Another embodiment described herein is a method for increasing homology directed repair (HDR) rates and reducing homology-independent integration in a programmable nuclease system comprising targeting a candidate editing target site locus with an active programmable nuclease system and a modified double stranded DNA HDR donor. In one aspect, the modified double stranded DNA HDR donor comprises a first homology arm region, an insert region, and a second homology arm region; wherein the first homology arm region and the second homology arm region comprises modifications to one or more nucleotides at or near the 5′-termini. In another aspect, the modified double stranded DNA HDR donor comprises modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region. In another aspect, the modified double stranded DNA HDR donor comprises at least one 2′-OME, 2′-F, or 2′-MOE modifications one or more nucleotides at or near the 5′-termini. In another aspect, the modified double stranded DNA HDR donor comprises one or more 2′-MOE modifications at or near the 5′-termini. In another aspect, the modified double stranded DNA HDR donor comprises universal primer sequences. In another aspect, the method further comprises one or more HDR enhancers. In another aspect, the modified double stranded DNA HDR donor improves homology directed repair efficiency and reduces homology-independent integration in a programmable nuclease system.

Another embodiment described herein is the use of modified double stranded DNA HDR donors for increasing homology directed repair (HDR) rates and reducing homology-independent integration in a programmable nuclease system, wherein the modified double stranded DNA HDR donor comprises a first homology arm region, an insert region, a second homology arm region; and optionally, one or more universal priming sequences; wherein the first homology arm region and the second homology arm region comprise modifications to one or more nucleotides at or near the 5′-termini. In one aspect, the modification comprises at least one 2′-OME, 2′-F, or 2′-MOE modifications one or more nucleotides at or near the 5′-termini of the double stranded DNA HDR donor.

Another embodiment described herein is a method for manufacturing a modified double stranded DNA HDR donor, the method comprising synthesizing an oligonucleotide comprising a first homology arm region, an insert region, a second homology arm region; and optionally, one or more universal priming sequences; wherein the first homology arm region and the second homology arm region comprise modifications to one or more nucleotides at or near the 5′-termini. In one aspect, the modification comprises at least one 2′-OME, 2′-F, or 2′-MOE modifications one or more nucleotides at or near the 5′-termini of the double stranded DNA HDR donor.

Another embodiment describe herein is a method for manufacturing a modified double stranded DNA HDR donor, the method comprising amplifying a target nucleic sequence comprising a first homology arm region, an insert region, a second homology arm region with one or more universal primers, wherein the universal priming sequences comprise modification to one or more nucleotides at or near the 5′-termini. In one aspect, the modification comprises at least one 2′-OME, 2′-F, or 2′-MOE modifications at one or more nucleotides at or near the 5′-termini of the universal primer.

### BRIEF DESCRIPTION OF THE DRAWINGS

FIG. 1A-B show schematics showing homology-independent and homology-dependent integration events when using a dsDNA HDR donor template for Cas9 directed cleavage (FIG. 1A) or endogenous double started breaks or Cas9 off-target cleavage (FIG. 1B). These homology-independent integration events lead to incorporation or duplication of homology arms at double stranded breaks introduced by programmable nucleases.

FIG. 2 shows the assessment of dsDNA donor integration via HDR or NHEJ pathways using modified linear dsDNA donors containing a 1 kb insert.

FIG. 3 shows the assessment of dsDNA donor integration via HDR or NHEJ pathways using modified linear dsDNA donors containing a 42 bp insert. Modifications were extended to multiple 2′-MOE ribonucleotides and internally placed 2′-MOE ribonucleotides.

FIG. 4 shows the assessment of dsDNA donor integration via HDR or NHEJ pathways using modified linear dsDNA donors containing a 42 bp insert. Cas9 guides targeting non-homologous sites were used to mimic off-target Cas9 cleavage.

FIG. 5 shows the assessment of dsDNA donor integration via HDR or NHEJ pathways using modified linear dsDNA donors containing a 42 bp insert. Modifications were extended to include additional 2′-modification. Cas9 guides targeting non-homologous sites were used to mimic off-target Cas9 cleavage.

FIG. 6A-B shows synthesis methods for hairpin blocked dsDNA HDR templates. Grey indicates a DNA hairpin composed of 2′-MOE ribonucleotides. Black indicates chemically synthesized unmodified DNA. White indicates a DNA template sequence with available primer binding sites. FIG. 6A shows that for short HDR inserts, hairpin blocked dsDNA donors can be generated by annealing two chemically synthesized ssDNA oligos containing the 5′-MOE hairpin. FIG. 6B shows that for longer HDR inserts, hairpin blocked dsDNA donors can be generated through PCR amplification. Primers with 5′-MOE hairpins can be used to amplify a target HDR template. The DNA polymerase should not be able to amplify through the MOE containing hairpin. After several cycles, a final dsDNA product containing MOE hairpins on both 5′-termini should be generated.

FIG. 7 shows an assessment of dsDNA donor integration via HDR or NHEJ pathways using donors with either a hairpin or a 1×MOE modified base at the 5′-termini. Donors contained 30 bp homology arms and mediated a 6 bp insert to introduce an EcoRI restriction site into the SERPCIN1 locus. Hairpins were composed of a 3 bp stem with a “TTTT” loop and contained either unmodified DNA bases (DNA-only) or 2′-MOE modified bases (MOE-modified). Hairpins were unligated for initial testing.

FIG. 8A-C show an assessment of dsDNA donor integration via HDR (FIG. 8A) or NHEJ (FIG. 8B) pathways using modified linear dsDNA donors. The ratio of HDR vs. blunt integration is shown in FIG. 8C. Donors were designed to mediate a 42 bp insertion at 4 genomic loci and were tested in 2 cell lines (n=8 per modification). Results are reported as the fold-change over the unmodified dsDNA donor for each site and cell line.

FIG. 9A shows an assessment of dsDNA donor integration via HDR or NHEJ pathways using modified linear dsDNA donors mediating a 300 bp, 500 bp, or 1 kb insert at two genomic loci. A long ssDNA donor targeting the SERPINC1 locus was provided for comparison. FIG. 9B shows comparison orthogonal analysis methods for assessment of insertion at the SERPINC1 locus. Long-read sequencing using the MinION™ system from Oxford Nanopore Technologies (ONT) was compared to amplicon length analysis where PCR amplicons from genomic DNA samples were run and quantified on a Fragment Analyzer.

FIG. 10 shows a schematic of dsDNA HDR donor template design comprising universal priming sequences. Hashed black indicates DNA sequence that is homologous between the genomic DNA target and the HDR donor (i.e., homology arms). Black indicates the desired insert DNA sequence. White indicates DNA sequence homologous to the universal priming sequences.

FIG. 11 shows an assessment of dsDNA donor integration via HDR or NHEJ pathways using modified linear dsDNA donors composed of a 500 bp insert flanked by 100 bp homology arms. Donors were synthesized with or without terminal universal priming sequences.

FIG. 12A-C show a visual assessment of HDR reads from 1×MOE donors using IGV. HDR reads from the EMX1 and SERPINC1 1×MOE dsDNA donors manufactured with the universal priming sequences were aligned against a reference containing either the correct HDR sequence (FIG. 12A) or the HDR sequence with the universal sequences (i.e., incorrect HDR) (FIG. 12B). For comparison, HDR reads from the 1×MOE dsDNA donors lacking universal sequences were aligned against the correct HDR reference (FIG. 12C). Within the IGV plots, individual reads are represented as thin horizontal lines. Individual nucleotides that do not correctly align to the reference (i.e., insertions, gaps, or mutations) are marked in black. The background error rate from the MinION™ sequencing can be assessed in FIG. 12C. A representation of the HDR reference is shown above each IGV panel. Solid black represents the desired 500 bp insert. Dashed areas represent sequence homologous to the 100 bp donor homology arms. Dotted areas represent the 30 bp universal priming sequences. Areas of interest are indicated by arrows. Misalignments against the incorrect HDR reference (FIG. 12B) are evident in every HDR read, indicating a lack of the 30 bp universal sequences after the repair. Panels for EMX1 donors represent approximately 500 reads. Panels for SERPINC1 donors represent approximately 3700 reads.

FIG. 13 shows an assessment of HDR rates when using either unmodified or 1×MOE modified dsDNA donor templates. Donors were designed to insert GFP at the N-or C-terminus of the target genes and contained 200 bp homology arms. Donors were generated with universal priming sequences. HDR rates were assessed by flow cytometry (reported as % GFP positive cells).

FIG. 14A-B show an assessment of yields when dsDNA HDR templates are manufactured with either universal primers or gene specific primers. Twelve sequences >500 bp and twelve sequences <500 bp were manufactured and PCR yields were assessed. Overall yields for each group are shown in FIG. 14A, while comparisons between templates with or without universal primers for each sequence are shown in FIG. 14B.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Preferred methods and materials are described below, although methods and materials similar or equivalent to those described herein can be used in practice or testing of the present invention.

As used herein, the terms “amino acid,” “nucleotide,” “polypeptide,” “polynucleotide,” and “vector” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or R) are used herein.

As used herein, the terms such as “include,” “including,” “contain,” “containing,” “having,” and the like mean “comprising.” The disclosure also contemplates other embodiments “comprising,” “consisting of,” and “consisting essentially of,” the embodiments, aspects, or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the phrase “an effective amount” of a compound described herein refers to an amount of the compound described herein that will elicit the biological response, for example, reduction or inhibition of an enzyme or a protein activity, or ameliorate symptoms, alleviate conditions, slow or delay disease progression, or prevent a disease, etc.

As used herein, the terms “inhibit,” “inhibition,” or “inhibiting” refer to the reduction or suppression of a given condition, symptom, or disorder, or disease, or a significant decrease in the baseline activity of a biological activity or process.

As used herein, the term “universal primer” refers to a sequence that does not have a known alignment to a target sequence. Universal primers permit the sequence independent amplification of target sequences.

Disclosed herein are methods and compositions of dsDNA donor templates for improving HDR efficiency and reducing blunt integration events. In various embodiments the disclosed methods and compositions allow for a reduction in homology-independent integration following genomic editing with programmable nucleases. In some embodiments bulky modifications are placed at the 5′-terminus of the linear dsDNA donor. In further embodiments bulky modifications are placed at or near the 5′ end of the linear dsDNA donor. Additionally, modifications may be placed at the 2′-position of the DNA (e.g., 2′-MOE, 2′-OME, or 2′-F nucleotides) of a nucleotide at or near the 5′-nucleotide or nucleotides of the dsDNA donor. These modifications demonstrate an improved efficacy at reducing homology-independent integration. Furthermore, this reduction does not seem to be mediated through increased donor stability, as other modifications that have the established ability to block nuclease degradation (PS, etc.) do not also reduce the blunt integration rate to the same extent as other 2′-modifications.

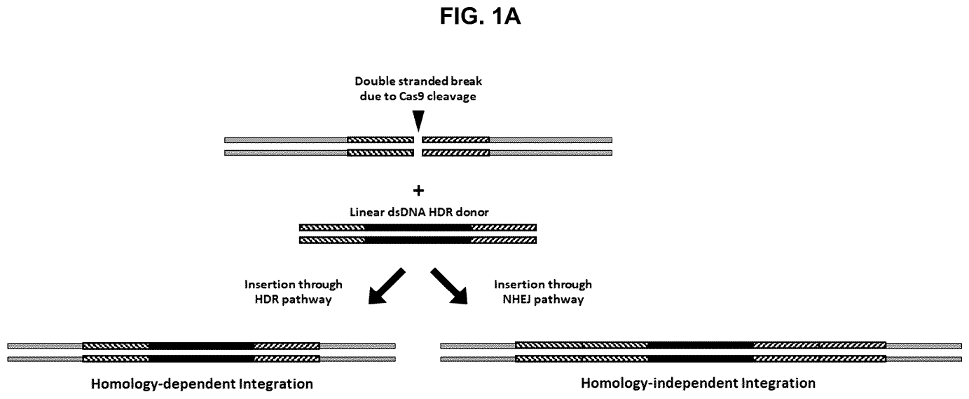

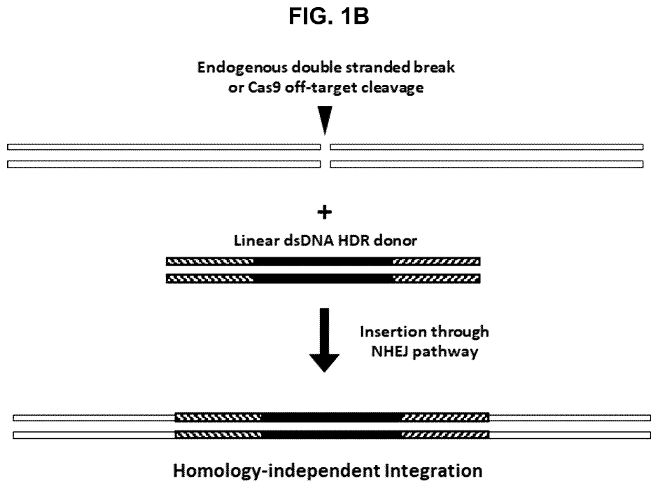

When homology-independent integration occurs at the on-target site, the entire donor is incorporated including the homology arms (FIG. 1) which leads to duplicated homology arms. FIG. 1 is a schematic of homology-independent (duplicated homology arms) and homology-dependent integration events when using a dsDNA HDR donor template. Light grey bars indicate the target genomic DNA sequence while white indicates a non-homologous genomic DNA sequence (either at an endogenous DSB or a Cas9 off-target site). Hashed black indicates DNA sequence that is homologous between the genomic DNA target and the HDR donor (i.e., homology arms). Black indicates the desired insert DNA sequence. FIG. 1A shows insertion of a dsDNA donor through the HDR or NHEJ repair pathways at the on-target Cas9 cleavage site. Insertion through the NHEJ pathway results in duplication of the donor homology arms. FIG. 1B shows insertion of a dsDNA through the NHEJ repair pathway at an endogenous DSB or at an off-target Cas9 cleavage site.

In some embodiments chemical modifications are introduced to the 5′-terminus of linear dsDNA donors. These chemical modifications are used to reduce the risk for NHEJ integration and improve their utility as repair templates in HDR experiments. In some embodiments bulky or large modifications are introduced to the 5′-terminal end of the dsDNA donor. In additional embodiments the modifications may be introduced to the terminal or 5′-DNA nucleotide of the dsDNA oligonucleotide. In some embodiments the modification may be introduced at or near the 5′-terminus of the dsDNA oligonucleotide. In some embodiments the DNA nucleotide or nucleotides at or near the 5′-terminus of the dsDNA oligonucleotide may be modified. In some embodiments, the modifications include biotin, phosphorothioate (PS), triethylene glycol (TEG), Locked Nucleic Acid (LNA, a 2′-oxygen-4′-carbon methylene linkage), hexaethylene glycol (Sp18), 1,3-propanediol (SpC3), 2′-O-methoxyethyl (MOE) ribonucleotides, 2′-O-methyl ribonucleotides (2′-OMe), 2′-fluoro (2′-F) nucleotides, or ribonucleotides. In some embodiments the modification is placed on the 5′-terminal nucleotide, the 5′-penulimate nucleotide, the 5′-antepenultimate (third) nucleotide, or a combination of the nucleotides at or near the 5′-terminus of the dsDNA donor. In additional embodiments the modification is placed at the 2′-position of the 5′-terminal nucleotide, the 5′-penulimate nucleotide, the 5′-antepenultimate (third) nucleotide, or a combination of the nucleotides at or near the 5′-terminus of the dsDNA donor. In yet an additional embodiment the modification is placed at the 2′-position of the 5′-terminal nucleotide, the 5′-penulimate nucleotide, the 5′-antepenultimate (third) nucleotide, or a combination of the nucleotides at or near the 5′-terminus of the dsDNA donor.

The use of 2′-modified ribonucleotides, particularly 2′-O-methoxyethyl (MOE), was found to give the optimal improvement when compared to biotin or other modifications. Additional experiments establishing the use of these modifications with donors mediating large insertions are described herein.

Further improvements to the manufacturing process of the dsDNA donors were evaluated. Universal priming sequences were selected to have no homology to common genomes (human, mouse, rat, zebrafish). Previous work by our group has established the utility of these priming sequences in cloning applications (i.e., highly efficient, reliable amplification). Significant improvements to (1) amplification success with a wide variety of sequences and (2) overall amplification yields can be achieved by incorporating these universal sequences into the donor manufacturing process. Described herein is testing of these universal sequences when placed flanking the complete HDR donor sequence. Due to the lowered risk of homology-independent integration when using the 5′-dsDNA modifications, these sequences do not adversely impact correct HDR rates with modified donors and are only rarely incorporated during blunt integration.

The methods and compositions disclosed herein are of dsDNA donor templates for use in improving HDR efficiency and reducing homology-independent events (blunt integration events or multimerization events). In various embodiments the disclosed methods and compositions allow for a reduction in homology-independent integration or increase in homology-dependent integration following genomic editing with programmable nucleases. In some embodiments bulky nucleotide modifications are placed at the 5′-terminus of the linear dsDNA donor. In additional embodiments modifications placed at the 2′-position of the nucleotide (e.g., 2′-MOE, 2′-OMe) of the 5′-terminal nucleotide or nucleotides near the 5′-terminus of the dsDNA demonstrate an improvement in efficacy at reducing homology-independent integration. Furthermore, this reduction does not seem to be mediated through increased donor stability, as other modifications that have the established ability to inhibit nuclease degradation (PS, etc.) do not also reduce the blunt integration rate to the same extent as other 2′-modifications.

In some embodiments chemical modifications are introduced to the 5′-terminus of linear dsDNA donors. These chemical modifications are used to reduce the risk for NHEJ integration and improve their utility as repair templates in HDR experiments. In some embodiments bulky or large modifications are introduced. In additional embodiments the modifications may be introduced near the 5′-terminus of the dsDNA oligonucleotide donor. In some embodiments the modification may be introduced at or near the 5′-terminus of the dsDNA oligonucleotide donor. In some embodiments the nucleotides at or near the 5′-terminus of the dsDNA oligonucleotide may be modified. In additional embodiments modifications include, but are not limited to: biotin (B); phosphorothioate (PS, *); triethylene glycol (TEG); Locked Nucleic Acid, e.g., a 2′-oxygen-4′-carbon methylene linkage (LNA); hexaethylene glycol (Sp18); 1,3-propanediol (SpC3); 2′-O-methoxyethyl (MOE) ribonucleotides, 2′-O-methyl ribonucleotides (2′-OMe), 2′-fluoro (2′-F) nucleotides, and ribonucleotides.

In further embodiments, the use of hairpin structures on the ends of the dsDNA donor similarly reduces blunt integration.

In one embodiment the end modified dsDNA donor templates would be suitable for use following introduction of double strand breaks by programmable nucleases. In further embodiments the programmable nucleases include transcription activator-like effector nucleases (TALENs), zinc fingers (ZFNs), or clustered, regularly interspaced, short palindromic repeat (CRISPR). In one embodiment, the programmable nuclease system is CRISPR. In one aspect, the programmable nuclease enzyme is CRISPR associated-9 (Cas9).

In one embodiment 5′-terminal modified dsDNA donors are generated by PCR amplification. Primers modified with biotin, phosphorothioate (PS) linkages, TEG, LNA, spacer 18 (SP18), C3 spacers (SpC3), or MOE are used to amplify insert regions and generate end modified dsDNA donors. In some embodiments the insert region is greater than 120 bp. In some embodiments the insert region is at least 1 kb insert regions. In some embodiments the insert region is greater than 1 kb, greater than 2 kb, greater than 3 kb, greater 4 kb, greater than 5 kb, greater than 6 kb, greater than 7 kb, greater than 8 kb, greater than 9 kb, or greater than 10 kb.

In additional embodiments, modifications at or near the 5′-terminus include biotin, phosphorothioate (PS), triethylene glycol (TEG), Locked Nucleic Acid, e.g., a 2′-oxygen-4′-carbon methylene linkage (LNA), hexaethylene glycol (Sp18), 1,3 propanediol (SpC3), 2′-O-methoxyethyl ribonucleotides (MOE), 2′-O-methyl ribonucleotides (2′-OMe), 2′-fluoro (2′-F) nucleotides, and ribonucleotides.

In further embodiments the modification at or near the 5′-terminus includes modifications of the 2′-position of the DNA nucleotide at or near the 5′-terminus of the double stranded DNA donor. In some embodiments the 2′-modification is 2′-MOE, 2′-OMe, or 2′-fluoro and the modification of the nucleotide occurs at or near the 5′-terminus of the double stranded DNA donor. In some embodiments the 5′-terminus modification is on the 5′-terminal nucleotide of the double stranded DNA donor. In additional embodiments the 5′-terminus modification is positioned at the 5′-terminal nucleotide, the 5′-penulimate nucleotide, the 5′-antepenultimate (third) nucleotide, or a combination of the nucleotides at or near the 5′-terminus of the dsDNA donor. In other embodiments the 5′-terminal modification is positioned at 5′-terminus modification is positioned at the 5′-terminal nucleotide, the 5′-penulimate nucleotide, or the 5′-antepenultimate (third) nucleotide. In yet another embodiment the 5′-terminal modification is a 2′-MOE modified ribonucleotide positioned at the terminal 5′-position, the penultimate nucleotide position from the 5′-terminus, the antepenultimate (third) nucleotide position from the 5′-terminus, or a combination thereof. In still a further embodiment the 5′-terminal modification is a 2′-MOE ribonucleotide positioned at the terminal 5′-position, the penultimate nucleotide position from the 5′-terminus, the antepenultimate (third) nucleotide position from the 5′-terminus, or a combination thereof.

In an additional embodiments HDR donors comprise homology arms on either side of an insert. The homology arms are complementary to the sequences flanking the double-stranded break introduced by the programmable nuclease. In some embodiments the homology arms vary in length from at least 20 nucleotides in length to 500 nucleotides in length. In some embodiments the homology arms are at least 40, 50, 60 70, 80, 90, 100, 150, 200, 300, 400, or 500 nucleotides in length. In some embodiments the homology arm length may be greater than 500 nucleotides in length. In additional embodiments the homology arms are preferably at least 40 nucleotides in length and more preferably at least 100 nucleotides in length.

In some embodiments the inserts are placed between homology arms. In some embodiments the inserts are greater than 20 nucleotides in length. In some embodiments the inserts are from at least 1 nucleotide in length to 4 kb in length. In some embodiments the inserts range from 1-2 kb in length. In some embodiments the inserts may be at least 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb in length. In yet an additional embodiment the insert may be 10 kb or longer in length.

In an additional embodiment HDR donor comprise homology arms on either side of an insert where the insert may include SNPs, MNPs, or deletions. In some embodiments the inserts are from at least 1 nucleotide in length to 4 kb in length. In some embodiments the inserts range from 1-2 kb in length. In some embodiments the inserts may be at least 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb in length. In yet an additional embodiment the insert may be 10 kb or longer in length.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% identical to (a) nucleotide sequences, or degenerate, homologous, or codon-optimized variants thereof; or (b) nucleotide sequences capable of hybridizing to the complement of any of the nucleotide sequences in (a).

By a polynucleotide having a nucleotide sequence at least, for example, 90-99% “identical” to a reference nucleotide sequence is intended that the nucleotide sequence of the polynucleotide be identical to the reference sequence except that the polynucleotide sequence can include up to about 10 to 1 point mutations, additions, or deletions per each 100 nucleotides of the reference nucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′-or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

In some embodiments the programmable nucleases (e.g., CRISPR enzyme) or components (e.g. gRNA) can be introduced into the cell using various approaches. Examples include plasmid or viral expression vectors (which lead to endogenous expression of either the enzyme, the gRNA, or both), delivery of the enzyme with separate gRNA/crRNA transfection, or delivery of the enzyme with the gRNA or crRNA as a ribonucleoprotein (RNP) complex.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

### REFERENCES

1. Chang et al., “Non-homologous DNA end joining and alternative pathways to double-strand break repair.” Nature Reviews Molecular Cell Biology 18:495-506 (2017).
2. Roth et al., “Reprogramming human T cell function and specificity with non-viral genome targeting,” Nature 559 (7714): 405-409 (2018).
3. Li et al., “Design and specificity of long ssDNA donors for CRISPR-based knock-in,” bioRxiv doi: 10.1101/178905 (2017).
4. Gutierrez-Triann et al., “Efficient single-copy HDR by 5′ modified long dsDNA donors,” eLife 2018;7:e39468; DOI: 10.7554/eLife.39468 (2018).
5. Canaj et al., “Deep profiling reveals substantial heterogeneity of integration outcomes in CRISPR knock-in experiments,” bioRxiv doi: 10.1101/841098 (2019).
6. Ghanta et al., “5′ Modifications Improve Potency and Efficacy of DNA Donors for Precision Genome Editing,” bioRxiv doi: 10.1101/354480 (2018).
7. Robinson et al., “Integrative Genomics Viewer,” Nature Biotechnology 29: 24-26 (2011).

### EMBODIMENTS

A1. A double stranded DNA homology directed repair (HDR) donor comprising: a first homology arm region, an insert region, and a second homology arm region; wherein the first homology arm region and the second homology arm region comprise modifications to one or more nucleotides at or near the 5′-termini.
A2. The double stranded DNA HDR donor of A1, wherein the modifications comprise: modifications to the 2′-position of one or more nucleotides at or near the 5′-terminus of the first homology arm region and modifications to the 2′-position of one or more nucleotides at or near the 5′-terminus of the second homology arm region.
A3. The double stranded DNA HDR donor of A1-A2, wherein the modifications comprise modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region.
A4 The double stranded DNA HDR donor of A1, wherein the modifications at or near the 5′-termini of the double stranded DNA HDR donor comprise one or more of: 2′-OME, 2′-F, or 2′-MOE.
A5. The double stranded DNA HDR donor of A1-A4, wherein the modifications at or near the 5′-termini of the double stranded DNA HDR donor comprise 2′-MOE.
A6 The double stranded DNA HDR donor of A1-A5, wherein the modification at or near the 5′-termini are non-template mismatches relative to a target DNA.
A7 The double stranded DNA HDR donor of A1-A6, wherein the first homology arm region and the second homology arm region are 40 to 150 nucleotides in length.
A8 The double stranded DNA HDR donor of A1-A7, wherein the first homology arm region and the second homology arm region are at least 100 nucleotides in length.
A9 The double stranded DNA HDR donor of A1-A8, wherein the double stranded DNA HDR donor further comprises universal primer sequences.
A10. The double stranded DNA HDR donor of A1-A9, wherein the insert region is greater than 100 bp.
A11. The double stranded DNA HDR donor of A1-A10, wherein the insert region is greater than 0.25 kb, greater than 0.5 kb, greater than 1 kb, greater than 2 kb, greater than 3 kb, greater 4 kb, greater than 5 kb, greater than 6 kb, greater than 7 kb, greater than 8 kb, greater than 9 kb, or greater than 10 kb.
A12 The double stranded DNA HDR donor of A1-A11, wherein the double stranded HDR donor comprises a hairpin at either the 5′-terminus or the 3′-terminus.
A13. The double stranded DNA HDR donor of A1-A12, wherein the double stranded HDR donor comprises a hairpin at both the 5′-terminus and the 3′-terminus.
A14. The double stranded DNA HDR donor of A1-A13, wherein the double stranded DNA HDR donor improves homology directed repair efficiency and reduces homology-independent integration in a programmable nuclease system.
B1. A programmable nuclease system comprising: a modified double stranded DNA homology directed repair (HDR) donor, a programmable nuclease enzyme, and a gRNA, wherein the gRNA molecule is capable of targeting the programmable nuclease molecule to a target nucleic acid.
B2. The programmable nuclease system of B1, wherein the modified double stranded DNA HDR donor comprises a first homology arm region, an insert region, and a second homology arm region; wherein the first homology arm region and the second homology arm region comprises modifications to one or more nucleotides at or near the 5′-termini.
B3. The programmable nuclease system of B1-B2, wherein the modified double stranded DNA HDR donor comprises modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region.
B4 The programmable nuclease system of B1-B3, wherein the modified double stranded DNA HDR donor comprises at least one 2′-OME, 2′-F, or 2′-MOE modifications one or more nucleotides at or near the 5′-termini.
B5. The programmable nuclease system of B1-B4, wherein the modified double stranded DNA HDR donor comprises one or more 2′-MOE modifications at or near the 5′-termini.
B6. The programmable nuclease system of B1-B5, wherein the modified double stranded DNA HDR donor comprises universal primer sequences.
B7. The programmable nuclease system of B1-B6, wherein the modified double stranded DNA HDR donor improves homology directed repair efficiency and reduces homology-independent integration in a programmable nuclease system.
B8. The programmable nuclease system of B1-B7, wherein the programmable nuclease system comprises one or more of transcription activator-like effector nucleases (TALENs), zinc fingers (ZFNs), or clustered, regularly interspaced, short palindromic repeat (CRISPR).
B9. The programmable nuclease system of B1-B8, wherein the programmable nuclease system is CRISPR.
B10. The programmable nuclease system of B1-B9, wherein the programmable nuclease enzyme is CRISPR associated-9 (Cas9).
B11. The programmable nuclease system of B1-B10, wherein the programmable nuclease system further comprises one or more HDR enhancers.
C1. A method for increasing homology directed repair (HDR) rates and reducing homology-independent integration in a programmable nuclease system comprising targeting a candidate editing target site locus with an active programmable nuclease system and a modified double stranded DNA HDR donor.
C1. The method of C1, wherein the modified double stranded DNA HDR donor comprises a first homology arm region, an insert region, and a second homology arm region; wherein the first homology arm region and the second homology arm region comprises modifications to one or more nucleotides at or near the 5′-termini.
C2. The method of C1, wherein the modified double stranded DNA HDR donor comprises modifications to the 2′-position of the 5′-terminal nucleotide, the 5′-penulimate nucleotide, the 5′-antepenultimate (third) nucleotide, or a combination of the nucleotides at or near the 5′-terminus of the first homology arm region and the second homology arm region.
C3. The method of C1-C2, wherein the modified double stranded DNA HDR donor comprises at least one 2′-OME, 2′-F, or 2′-MOE modifications one or more nucleotides at or near the 5′-termini.
C4. The method of C1-C3, wherein the modified double stranded DNA HDR donor comprises one or more 2′-MOE modifications at or near the 5′-termini.
C5. The method of C1-C4, wherein the modified double stranded DNA HDR donor comprises universal primer sequences.
C6. The method of C1-C5, wherein the method further comprises one or more HDR enhancers.
C7. The method of C1-C6, wherein the modified double stranded DNA HDR donor improves homology directed repair efficiency and reduces homology-independent integration in a programmable nuclease system.
D1. A use of modified double stranded DNA HDR donors for increasing homology directed repair (HDR) rates and reducing homology-independent integration in a programmable nuclease system, wherein the modified double stranded DNA HDR donor comprises a first homology arm region, an insert region, a second homology arm region; and optionally, one or more universal priming sequences; wherein the first homology arm region and the second homology arm region comprise modifications to one or more nucleotides at or near the 5′-termini.
D2. The use of D1, wherein the modification comprises at least one 2′-OME, 2′-F, or 2′-MOE modifications one or more nucleotides at or near the 5′-termini of the double stranded DNA HDR donor.
E1. A method for manufacturing a modified double stranded DNA HDR donor, the method comprising synthesizing a first oligonucleotide comprising a first homology arm region, an insert region, a second homology arm region; and optionally, one or more universal priming sequences, synthesizing a second complementary oligonucleotide sequence, and hybridizing the first oligonucleotide and second oligonucleotide sequence; wherein the first homology arm region and the second homology arm region comprise modifications to one or more nucleotides at or near the 5′-termini.
E2. The method of E1, wherein the modification comprises at least one 2′-OME, 2′-F, or 2′-MOE modifications one or more nucleotides at or near the 5′-termini of the double stranded DNA HDR donor.
F1. A method for manufacturing a modified double stranded DNA HDR donor, the method comprising amplifying a target nucleic sequence comprising a first homology arm region, an insert region, a second homology arm region with one or more universal primers, wherein the universal priming sequences comprise modification to one or more nucleotides at or near the 5′-termini.
F2. The method of F1, wherein the modification comprises at least one 2′-OME, 2′-F, or 2′-MOE modifications at one or more nucleotides at or near the 5′-termini of the universal primer.

### EXAMPLES

### Example 1

HDR Rates Are Increased, and NHEJ Insertions Are Reduced With Modified dsDNA Donors.

Initial tests were performed to compare the homology-independent (i.e., blunt) integration relative to HDR insertion rates of unmodified linear dsDNA, donors containing 5′-biotin modification, or donors with alternative modifications on or near the 5′-termini. dsDNA donors were generated by PCR amplification of a plasmid containing a 1 kb insert with 100 bp of flanking homology arms targeting the human SERPINC1 gene (100-1000-100; SEQ ID NO: 1; see Table 1 for amplification primer sequences; SEQ ID NO: 2-21). Amplification primers were designed with either unmodified sequence or the indicated modifications. Purified dsDNA donors were delivered at 100 nM (1 μg) in a final volume of 28 μL nucleofection buffer with 2 μM Cas9 V3™ RNP (IDT, Coralville, lowa) targeting SERPINC1 into 3.5×10⁵ HEK-293 cells using Lonza nucleofection (Lonza, Basel, Switzerland). The SC1 (SERPINC1) protospacer sequence used can be found in Table 1 (SEQ ID NO: 22). Cells were lysed after 48 hours using QuickExtract™ DNA extraction solution (Lucigen, Madison, WI). HDR and blunt integration rates were assessed by digital-droplet PCR (ddPCR) (Bio-Rad, Hercules, CA) using PCR assays with primers flanking the junction between the target DNA and insert (Table 1; SEQ ID NO: 23-27). Both HDR and blunt junction assays contained one primer external to the homology arm sequence to avoid amplification from non-integrated donor. The HDR assay probe (SEQ ID NO: 25) covered the junction of the target site and insert sequence. The blunt assay probe (SEQ ID NO: 27) covered the junction between the target site and integrated homology arm sequence.

TABLE 1
Sequences of primers, probes, crRNAs, and
templates used in Example 1.

| SEQ | | |
|---|---|---|
| ID | | |
| NO. | Name | Sequence |
| SEQ | SC1 | GATTGCCTCAGATCACACTATCTCCACTTGCCCAGCCCT |
| ID | 100- | GTGGAAGATTAGCGGCCATGTATTCCAATGTGATAGGAA |
| NO: | 1000- | CTGTAACCTCTGGAAAAAGGTACGAATTCGAGGGCAGAG |
| 1 | 100 | GCAGTCTGCTGACATGCGGTGACGTGGAAGAGAATCCCG |
| | donor | GCCCTTCTAGAATGGTTAGCAAGGGCGAGGAGCTGTTCA |
| | | CCGGGGTGGTGCCCATCCTGGTCGAGCTGGACGGCGACG |
| | | TAAACGGCCACAAGTTCAGCGTGTCCGGCGAGGGCGAGG |
| | | GCGATGCCACCTACGGCAAGCTGACCCTGAAGTTCATCT |
| | | GCACCACCGGCAAGCTGCCCGTGCCCTGGCCCACCCTCG |
| | | TGACCACCCTGACCTACGGCGTGCAGTGCTTCAGCCGCT |
| | | ACCCCGACCACATGAAGCAGCACGACTTCTTCAAGTCCG |
| | | CCATGCCCGAAGGCTACGTCCAGGAGCGCACCATCTTCT |
| | | TCAAGGACGACGGCAACTACAAGACCCGCGCCGAGGTGA |
| | | AGTTCGAGGGCGACACCCTGGTGAACCGCATCGAGCTGA |
| | | AGGGCATCGACTTCAAGGAGGACGGCAACATCCTGGGGC |
| | | ACAAGCTTGAGTACAACTACAACAGCCACAACGTCTATA |
| | | TCATGGCCGACAAGCAGAAGAACGGCATCAAGGTGAACT |
| | | TCAAGATCCGCCACAACATCGAGGACGGCAGCGTGCAGC |
| | | TCGCCGACCACTACCAGCAGAACACCCCCATCGGCGACG |
| | | GCCCCGTGCTGCTGCCCGACAACCACTACCTGAGCACCC |
| | | AGTCCGCCCTGAGCAAAGACCCCAACGAGAAGCGCGATC |
| | | ACATGGTCCTGCTGGAGTTCGTGACCGCCGCCGGGATCA |
| | | CTCTCGGCATGGACGAGCTGTACAAGTAACTGTGCCTTC |
| | | TAGTTGCCAGCCATCTGTTGTTTGCCCCTCCCCCGTGCC |
| | | TTCCTTGACCCTGGAAGGTGCCACTCCCACTGTCCTTTC |
| | | CTAATAAAATGAGGAAATTGCATCGCATTGTCTGAGTAG |
| | | GTGTCATTCTATTCTGGGGGGTGGGGTGGGGCAGGACAG |
| | | CAAGGGGGAGGATTGGGAAGACAATAGCAGGCATGCTGG |
| | | GGATGCGGTGGGCTCTATGGCGGTACCAGAGGGGTGAGC |
| | | TTTCCCCTTGCCTGCCCCTACTGGGTTTTGTGACCTCCA |
| | | AAGGACTCACAGGAATGACCTCCAACACCTTTGAGAAGA |
| | | CCAGGCCCTC |
| SEQ | SC1 | GATTGCCTCAGATCACACTATCTCC |
| ID | 100 | |
| NO: | Fwd | |
| 2 | unmod | |
| SEQ | SC1 | GAGGGCCTGGTCTTCTCAAAG |
| ID | 100 | |
| NO: | Rev | |
| 3 | unmod | |
| SEQ | SC1 | B-GATTGCCTCAGATCACACTATCTCC |
| ID | 100 | |
| NO: | Fwd | |
| 4 | Biotin | |
| SEQ | SC1 | B-GAGGGCCTGGTCTTCTCAAAG |
| ID | 100 | |
| NO: | Rev | |
| 5 | Biotin | |
| SEQ | SC1 | G*A*TTGCCTCAGATCACACTATCTCC |
| ID | 100 | |
| NO: | Fwd | |
| 6 | 2 × PS | |
| SEQ | SC1 | G*A*GGGCCTGGTCTTCTCAAAG |
| ID | 100 | |
| NO: | Rev | |
| 7 | 2 × PS | |
| SEQ | SC1 | G*A*T*TGCCTCAGATCACACTATCTCC |
| ID | 100 | |
| NO: | Fwd | |
| 8 | 3 × P5 | |
| SEQ | SC1 | G*A*G*GGCCTGGTCTTCTCAAAG |
| ID | 100 | |
| NO: | Rev | |
| 9 | 3 × PS | |
| SEQ | SC1 | G*A*T*T*G*C*CTCAGATCACACTATCTCC |
| ID | 100 | |
| NO: | Fwd | |
| 10 | 6 × PS | |
| SEQ | SC1 | G*A*G*G*G*C*CTGGTCTTCTCAAAG |
| ID | 100 | |
| NO: | Rev | |
| 11 | 6 × PS | |
| SEQ | SC1 | TEG-GATTGCCTCAGATCACACTATCTCC |
| ID | 100 | |
| NO: | Fwd | |
| 12 | TEG | |
| SEQ | SC1 | TEG-GAGGGCCTGGTCTTCTCAAAG |
| ID | 100 | |
| NO: | Rev | |
| 13 | TEG | |
| SEQ | SC1 | +GATTGCCTCAGATCACACTATCTCC |
| ID | 100 | |
| NO: | Fwd | |
| 14 | LNA | |
| SEQ | SC1 | +GAGGGCCTGGTCTTCTCAAAG |
| ID | 100 | |
| NO: | Rev | |
| 15 | LNA | |
| SEQ | SC1 | Sp18-GATTGCCTCAGATCACACTATCTCC |
| ID | 100 | |
| NO: | Fwd | |
| 16 | Sp18 | |
| SEQ | SC1 | Sp18-GAGGGCCTGGTCTTCTCAAAG |
| ID | 100 | |
| NO: | Rev | |
| 17 | Sp18 | |
| SEQ | SC1 | SpC3-GATTGCCTCAGATCACACTATCTCC |
| ID | 100 | |
| NO: | Fwd | |
| 18 | SpC3 | |
| SEQ | SC1 | SpC3-GAGGGCCTGGTCTTCTCAAAG |
| ID | 100 | |
| NO: | Rev | |
| 19 | SpC3 | |
| SEQ | SC1 | MGATTGCCTCAGATCACACTATCTCC |
| ID | 100 | |
| NO: | Fwd | |
| 20 | MOE | |
| SEQ | SC1 | MGAGGGCCTGGTCTTCTCAAAG |
| ID | 100 | |
| NO: | Rev | |
| 21 | MOE | |
| SEQ | SC1- | ACCTCTGGAAAAAGGTAAGA |
| ID | 166S | |
| NO: | guide | |
| 22 | proto- | |
| | spacer | |
| SEQ | SC1 | AGAACCAGTTTTCAGGCGG |
| ID | ddPCR | |
| NO: | For | |
| 23 | | |
| SEQ | SC1 | ACCGCATGTCAGCAGAC |
| ID | ddPCR | |
| NO: | HDR | |
| 24 | Rev | |
| SEQ | SC1 | FAM-TGGAAAAAG-ZEN-GTACGAATTCGAGGGCA-FQ |
| ID | ddPCR | |
| NO: | HDR | |
| 25 | Probe | |
| SEQ | SC1 | CGCTAATCTTCCACAGGG |
| ID | ddPCR | |
| NO: | Blunt | |
| 26 | Rev | |
| SEQ | SC1 | FAM-TCTGGAAAA-ZEN-AGGTAGATTGCCTCAGATCA- |
| ID | ddPCR | FQ |
| NO: | Blunt | |
| 27 | Probe | |

DNA is uppercase; B- is a 5′-biotin moiety; phosphorothioate (PS) modified linkages are shown with an asterisk (*); triethylene glycol spacer is indicated by an uppercase TEG; 5′-locked ribonucleotides (2′-oxygen-4′-carbon methylene linkage) are shown as a + before the modified ribonucleotide; hexaethylene glycol spacer 18 is shown as Sp18; 1,3-propanediol spacer is shown as SpC3; 2′-O-methoxyethyl modified ribonucleotides are shown with an uppercase M preceeding the modified ribonucleotide; 2′-O-methyl modified ribonucleotides are shown with shown with a lower-case m preceeding the modified ribonucleotide; FAM is 5,6 fluorescein dye; FQ is Iowa Black ™ FQ fluorescent quencher; and ZEN is ZEN ™
fluorescent quencher. SC1 is SERPINC1. All primers, probes and templates were synthesized by IDT (Coralville, IA).

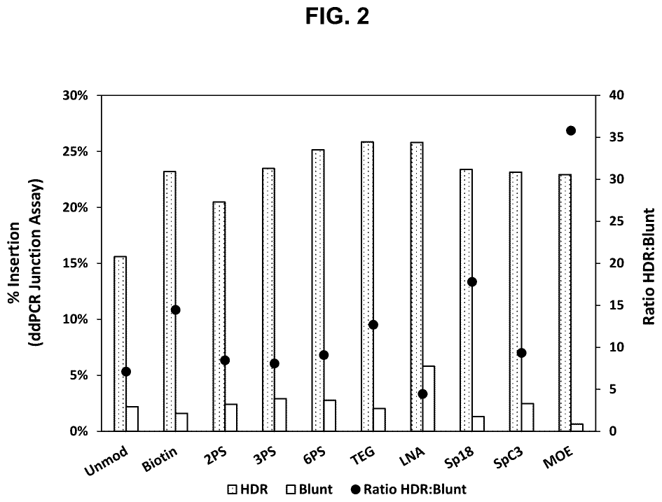

dsDNA donors containing known nuclease resistant modifications, such as phosphorothioate linkages (2×PS, 3×PS, or 6×PS) or an LNA nucleotide on the 5′-terminus did not improve the HDR:Blunt ratio above unmodified (unmod) donors, as the modifications increased the rates for both HDR and blunt integration (FIG. 2). 5′-modifications (Biotin, TEG, Sp18, and SpC3) on the donor resulted in increased HDR rates with varying degrees of decreased blunt integration. Of these donors, TEG, Biotin, and Sp18 showed increases in the HDR:Blunt ratio (1.8-, 2.0-, and 2.5-fold improvements over unmodified, respectively). See FIG. 2. A donor containing a 2′-O-methoxy-ethyl (2′-MOE) modified ribonucleotide at both of the 5′-termini gave the greatest increase in the HDR: Blunt ratio (5.0-fold improvement over the unmodified donor). See FIG. 2. The HDR rate was similar between the 2′-MOE modification and the other modified donors, suggesting the increased stability alone was not responsible for the increased HDR: Blunt ratio. Furthermore, as stated earlier, the LNA modified donor and the PS-modified donors did not increase the HDR: Blunt rate, indicating that decreased blunt integration is likely not arising from increased nuclease resistance of the template. It also demonstrates that using any 2′-modified ribonucleotide near the 5′-termini of the donor is insufficient to lower blunt integration, and that 2′-MOE modified templates are the most competent for this activity amongst the modifications tested here.

### Example 2

### 5′-Modifications Demonstrate Lower Off-Target Integration When Using Shorter Donor Templates.

Homology-independent integration rates depend on the total donor length, with blunt insertion increasing with decreased donor size. To determine whether 5′-terminal modification would reduce blunt insertion rates with a smaller 42 bp insert (compared with the 1 kb insert tested in Example 1), modified dsDNA donors were generated targeting the SERPINC1 locus described in Example 1 (SC1-166S; SEQ ID NO: 22). Donors consisted of a 42 bp insert containing an EcoRI restriction site with 40 bp homology arms (SC1 40-42-40; SEQ ID NO: 28) and were generated by PCR amplification of a plasmid containing the 42 bp insert with 100 bp of flanking homology arms (see Table 2 for amplification primer sequences; SEQ ID NO: 29-44).

Three modifications (Biotin, Sp18, and MOE) from Example 1 were selected for additional testing, while a 6×PS modification was included as a moderately performing control. Donors with three 2′-MOE ribonucleotides (3×MOE) on the 5′-termini were also included to determine if blunt integration could be further reduced by additional modified residues. A 2′-MOE ribonucleotide was also tested at varying distances from the 5′-termini (Int MOE-3 and-5, with a 2′-MOE positioned 3 or 5 nucleotides from the 5′-terminus; SEQ ID NO: 41-44). Modified and unmodified donors were delivered at 500 nM (1.1 μg) with 2 μM Cas9 V3™ (IDT, Coralville, IA) RNP targeting the SERPINC1 locus into 3.5×10⁵ HEK-293 cells in a 28 μL final volume using Lonza nucleofection (Lonza, Basel Switzerland). DNA was extracted after 48 hours using QuickExtract™ DNA extraction solution (Lucigen, Madison, WI). Integration rates were assessed by RFLP using EcoRI digestion, using a Fragment Analyzer™ machine for band quantification (Advanced Analytical, Ames, IA). HDR and blunt integration events could be distinguished by a 40 bp size difference due to the homology arm duplication.

TABLE 2
Sequences of primers and templates used
in Example 2. SEQ ID NO: 22 used for
crRNA (Table 1).

| SEQ ID NO. | Name | Sequence |
|---|---|---|
| SEQ ID NO: 28 | SC1 40-42-40 | ATTCCAATGTGA |
| | donor | TAGGAACTGTAA |
| | | CCTCTGGAAAAA |
| | | GGTAGAATTCTT |
| | | AGCTCTGTTTAC |
| | | GTCCCAGCGGGC |
| | | ATGAGAGTAAAG |
| | | AGGGGTGAGCTT |
| | | TCCCCTTGCCTG |
| | | CCCCTACTGGGT |
| | | TT |
| SEQ ID NO: 29 | SC1 40 Fwd | ATTCCAATGTGA |
| | unmod | TAGGAACTGTAA |
| | | CC |
| SEQ ID NO: 30 | SC1 40 Rev | AAACCCAGTAGG |
| | unmod | GGCAGGC |
| SEQ ID NO: 31 | SC1 40 Fwd | MATTCCAATGTG |
| | 1 × MOE | ATAGGAACTGTA |
| | | ACC |
| SEQ ID NO: 32 | SC1 40 Rev | MAAACCCAGTAG |
| | 1 × MOE | GGGCAGGC |
| SEQ ID NO: 33 | SC1 40 Fwd | MAMTMTCCAATG |
| | 3 × MOE | TGATAGGAACTG |
| | | TAACC |
| SEQ ID NO: 34 | SC1 40 Rev | MAMAMACCCAGT |
| | 3 × MOE | AGGGGCAGGC |
| SEQ ID NO: 35 | SC1 40 Fwd | A*T*T*C*C*A* |
| | 6 × PS | ATGTGATAGGAA |
| | | CTGTAACCTCTG |
| SEQ ID NO: 36 | SC1 40 Rev | A*A*A*C*C*C* |
| | 6 × PS | AGTAGGGGCAGG |
| | | C |
| SEQ ID NO: 37 | SC1 40 Fwd | B-ATTCCAATGT |
| | Biotin | GATAGGAACTGT |
| | | AACC |
| SEQ ID NO: 38 | SC1 40 Rev | B-AAACCCAGTA |
| | Biotin | |
| | | GGGGCAGGC |
| SEQ ID NO: 39 | SC1 40 Fwd | Sp18-ATTCCAA |
| | Sp18 | TGTGATAGGAAC |
| | | TGTAACC |
| SEQ ID NO: 40 | SC1 40 Rev | Sp18-AAACCCA |
| | Sp18 | GTAGGGGCAGGC |
| SEQ ID NO: 41 | SC1 40 Fwd | ATMTCCAATGTG |
| | IntMOE(-3) | ATAGGAACTGTA |
| | | ACCTCTG |
| SEQ ID NO: 42 | SC1 40 Rev | AAMACCCAGTAG |
| | IntMOE(-3) | GGGCAGGC |
| SEQ ID NO: 43 | SC1 40 Fwd | ATTCMCAATGTG |
| | IntMOE(-5) | ATAGGAACTGTA |
| | | ACCTCTG |
| SEQ ID NO: 44 | SC1 40 Rev | AAACMCCAGTAG |
| | IntMOE(-5) | GGGCAGGC |
| SEQ ID NO: 45 | SC1 RFLP For | CTTGTCCCTCTT |
| | | TGCCTTCTCT |
| SEQ ID NO: 46 | SC1 RFLP Rev | GGGTGGATCTGA |
| | | GTGGAAGAAA |

DNA is uppercase; B- is a 5′-biotin moiety; phosphorothioate (PS) modified linkages are shown with an asterisk (*); hexaethylene glycol spacer is shown as Sp18; and 2′-O-methoxyethyl modified ribonucleotides are shown with an uppercase M preceeding the modified ribonucleotide. SC1 is SERPINC1. All primers, guides, and templates were
synthesized by IDT (Coralville, IA).

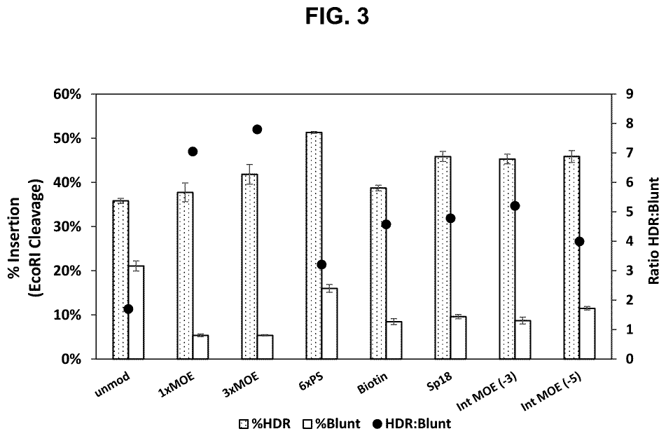

1×MOE and 3×MOE modified donors resulted in the greatest improvement in the HDR:Blunt ratio (4.1-and 4.6-fold improvement over unmod respectively). See FIG. 3. As previously observed, 6×PS, biotin, and Sp18 yielded some improvement (1.9-, 2.7-, and 2.8-fold improvement over unmod respectively) but did not reduce blunt integration to the same extent as the MOE modified donors. See FIG. 3. Interestingly, the position of the 2′-MOE ribonucleotide within the donor did slightly impact its utility for reducing blunt integration. Shifting the MOE ribonucleotide either 3-or 5-nucleotides from the 5′-termini of the donor resulted in only a 3.1-or 2.4-fold improvement in the HDR:Blunt ratio compared to unmodified donor. See FIG. 3. As such, a user skilled in the art would predict that a 2′-MOE ribonucleotide placed within 2-3 nucleotides of the 5′-termini of a donor template would yield a large reduction in NHEJ-mediated insertion.

### Example 3

### 2′-MOE Modifications Lower Integration at Non-Homologous Double Strand Breaks

In addition to blunt integration at the targeted cleavage site, double-strand donors can potentially integrate at any other double-stranded break in the genome, including off-target Cas9 cleavage sites and endogenous breaks in dsDNA. To assess the impact of the 2′-MOE modification on donor integration at potential non-homologous DSBs, dsDNA donors with either unmodified or modified 5′-termini (unmod, 1×MOE, or 6×PS) were generated by PCR amplification (see Table 3 for amplification primer sequences; SEQ ID NO: 48-53) and co-delivered with Cas9 complexed with either the target gRNA (SC1-166S; SEQ ID NO: 22) or a mock “off-target” gRNA with no homology to the donor (AAVS1-670AS; SEQ ID NO: 54; HPRT 38087; SEQ ID NO: 55).

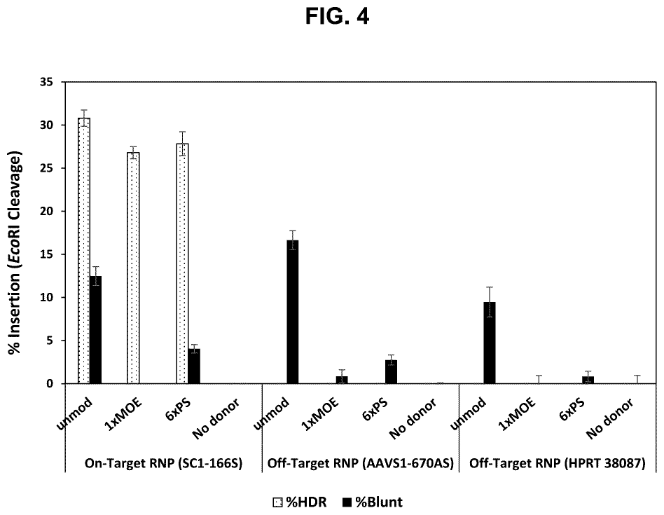

Donors consisted of a 42 bp insert containing an EcoRI restriction site and 50 bp homology arms targeting the SERPINC1 locus (SC1 50-42-50; SEQ ID NO: 47). Donors were delivered at a 100 nM dose (0.3 μg) with 2 μM Cas9 V3™ RNP (IDT, Coralville, IA) and 2 μM Alt-R™ Cas9 Electroporation Enhancer™ into 3×10⁵ K562 cells in a final 28 μL volume by Lonza nucleofection (Lonza, Basel Switzerland). DNA was extracted after 48 hrs using QuickExtract™ DNA extraction solution (Lucigen, Madison, WI). Integration rates were assessed by RFLP using EcoRI digestion, with each band quantified on a Fragment Analyzer™ machine (Advanced Analytical, Ames, IA). HDR and blunt integration events could be distinguished by a 50 bp size difference due to the homology arm duplication (FIG. 4).

TABLE 3
Sequences of primers, crRNA guides,
and templates used in Example 3.

| SEQ ID NO. | Name | Sequence |
|---|---|---|
| SEQ ID NO: 47 | SC2 | GCGGCCATGTATTCCAA |
| | 50-42-50 | TGTGATAGGAACTGTAA |
| | donor | CCTCTGGAAAAAGGTAG |
| | | AATTCTTAGCTCTGTTT |
| | | ACGTCCCAGCGGGCATG |
| | | AGAGTAAAGAGGGGTGA |
| | | GCTTTCCCCTTGCCTGC |
| | | CCCTACTGGGTTTTGTG |
| | | ACCTCC |
| SEQ ID NO: 48 | SC1 50 Fwd | GCGGCCATGTATTCCAA |
| | unmod | TGTG |
| SEQ ID NO: 49 | SC1 50 Rev | GGAGGTCACAAAACCCA |
| | unmod | GTAGG |
| SEQ ID NO: 50 | SC1 50 Fwd | MGCGGCCATGTATTCC |
| | 1 × MOE | AATGTG |
| SEQ ID NO: 51 | SC1 50 Rev | MGGAGGTCACAAAACCC |
| | 1 × MOE | AGTAGG |
| SEQ ID NO: 52 | SC1 50 Fwd | G*C*G*G*C*C*ATGTAT |
| | 6 × PS | TCCAATGTG |
| SEQ ID NO: 53 | SC1 50 Rev | G*G*A*G*G*T*CACAAA |
| | 6 × PS | ACCCAGTAGG |
| SEQ ID NO: 54 | AAVS1-670A5 | CCTCTAAGGTTTGCTTA |
| | guide | CGA |
| | protospacer | |
| SEQ ID NO: 55 | HPRT 38087 | AATTATGGGGATTACTA |
| | guide | GGA |
| | protospacer | |
| SEQ ID NO: 56 | AAVS1 RFLP | GCCAAGGACTCAAACCC |
| | Fwd | AGA |
| SEQ ID NO: 57 | AAVS1 RFLP | CCCCGTTCTCCTGTGGA |
| | Rev | TTC |
| SEQ ID NO: 58 | HPRT RFLP | AAGAATGTTGTGATAAA |
| | Fwd | AGGTGATGCT |
| SEQ ID NO: 59 | HPRT RFLP | ACACATCCATGGGACTT |
| | Rev | CTGCCTC |

DNA is uppercase; phosphorothioate (PS) modified linkages are shown with an asterisk (*); hexaethylene glycol spacer is shown as Sp18; and 2′-O-methoxyethyl modified ribonucleotides are shown with an uppercase M preceeding the modified ribonucleotide. SC1 is SERPINC1. All primers, guides, and templates were synthesized by IDT (Coralville, IA).

Blunt integration rates >9% were observed for unmodified dsDNA at the on-target Cas9 site and both “off-target” Cas9 sites. See FIG. 4. Reduced blunt integration (<1%) was observed with the 2′-MOE modified donor, demonstrating that 2′-MOE modifications can also reduce NHEJ-mediated insertions at non-homologous DSBs. See FIG. 4. As previously observed, a 6×PS modification resulted in moderate decrease in blunt integration.

### Example 4

### Reduction of Off-Target Integration is Not the Result of Increased Nuclease Protection, and Specific 2′-Modifications Are Required For Efficient Reduction

To determine whether the ability of 2′-MOE modification to reduce blunt integration was specific or a general function of modifications at the 2′-position of the 5′-most nucleotides, additional 2′-modifications were tested (RNA, LNA, 2′-OMe, or 2′-F), as well as a non-template 2′-MOE ribonucleotide (SEQ ID NO: 70-71) on the 5′-termini. (Non-template ribonucleotide defined as non-homologous to the target DNA sequence.) Donors consisted of the sequence previously described in Example 2 (SC1 40-42-40; SEQ ID NO: 28), a 42 bp insert containing an EcoRI restriction site and with 40 bp homology arms targeting the SerpinC1 locus. Donors were generated by PCR amplification as previously described (primer sequences unique to Example 4 listed in Table 4; SEQ ID NO: 60-71).

TABLE 4
Sequences of primers, probes, crRNAs,
and templates used in Example 4.

| SEQ ID NO. | Name | Sequence |
|---|---|---|
| SEQ ID NO: 60 | SC1 40 Fwd | MA*T*TCCAATGTGA |
| | 1 × MOE 2 × PS | TAGGAACTGTAACC |
| SEQ ID NO: 61 | SC1 40 Rev | MA*A*ACCCAGTAG |
| | 1 × MOE 2 × PS | GGGCAGGC |
| SEQ ID NO: 62 | SC1 40 Fwd | aTTCCAATGTGATA |
| | 5′-RNA | GGAACTGTAACC |
| SEQ ID NO: 63 | SC1 40 Rev | aAACCCAGTAGGG |
| | 5′-RNA | GCAGGC |
| SEQ ID NO: 64 | SC1 40 Fwd | +ATTCCAATGTGATAG |
| | LNA | GAACTGTAACC |
| SEQ ID NO: 65 | SC1 40 Rev | +AAACCCAGTAGGG |
| | LNA | GCAGGC |
| SEQ ID NO: 66 | SC1 40 Fwd | mATTCCAATGTGATAG |
| | 2′-OMe | GAACTGTAACC |
| SEQ ID NO: 67 | SC1 40 Rev | mAAACCCAGTAGGGGC |
| | 2′-OMe | AGGC |
| SEQ ID NO: 68 | SC1 40 Fwd | fATTCCAATGTGATAG |
| | 2′-F | GAACTGTAACC |
| SEQ ID NO: 69 | SC1 40 Rev | fAAACCCAGTAGGGGC |
| | 2′-F | AGGC |
| SEQ ID NO: 70 | SC1 40 Fwd Non- | MGATTCCAATGTGATA |
| | template 2′-MOE | GGAACTGTAACC |
| SEQ ID NO: 71 | SC1 40 Rev Non- | MGAAACCCAGTAGGGG |
| | template 2′-MOE | CAGGC |
| SEQ ID NO: 72 | TNPO3 gRNA | TGCCCTGGTAAC |
| | protospacer | GGCCAAAG |
| SEQ ID NO: 73 | TNPO3 RFLP | TCGGACAGAAAGG |
| | Fwd | CATTCACA |
| SEQ ID NO: 74 | TNPO3 RFLP | CAACGGCAAAGGG |
| | Rev | AGAACTTAAAC |

DNA is uppercase; RNA is lowercase; locked nucleic acids are shown as a + preceeding the modified nucleotide; 2′-O-methoxyethyl modified ribonucleotides are shown with an uppercase M preceeding the modified ribonucleotide; 2′-O-methyl modified ribonucleotides are shown with a lower-case m preceeding the modified ribonucleotide; 2′-fluoro modified ribonucleotides are shown with a lowercase f preceeding the modified ribonucleotide; and non-templated 2′-MOE modified ribonucleotides are shown underlined. SC1 is SERPINC1. All primers and templates were synthesized by IDT (Coralville, IA).

dsDNA donors were co-delivered with Cas9 complexed with either the target gRNA (SC1-166S; SEQ ID NO: 22) or a gRNA with no homology to the donor (TNPO3; SEQ ID NO: 72). Donors were delivered at a 250 nM dose (0.6 μg) with 2 μM Cas9 V3 RNP into HEK-293 cells in a final 28 μL volume by Lonza nucleofection (Lonza, Basel Switzerland). DNA was extracted after 48 hrs using QuickExtract™ DNA extraction solution (Lucigen, Madison, WI). Integration rates were assessed by RFLP using EcoRI, with each band quantified on a Fragment Analyzer™ machine (Advanced Analytical, Ames, IA).

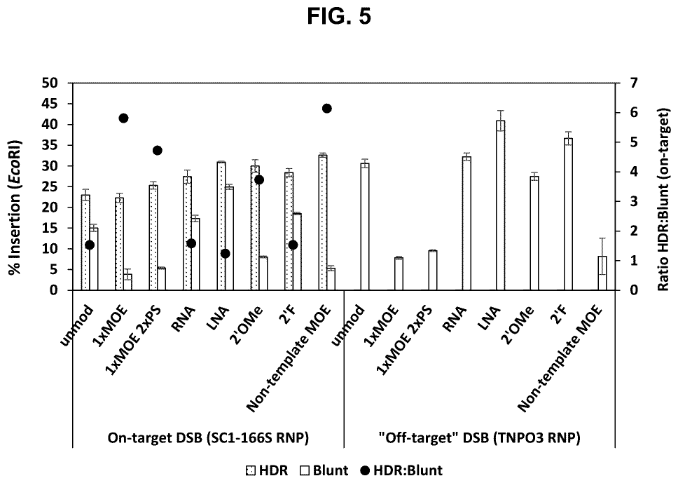

While HDR was either not impacted or slightly boosted by the various modifications, blunt integration was decreased at both the on-target and off-target DSBs whenever a 2′-MOE modification was present (FIG. 5). In contrast, most of the additional 2′-modifications either did not impact or increased the blunt integration rate. The 2′-OMe modification did reduce the blunt integration rate at the on-target DSB, but not to the same extent as the 2′-MOE modifications. See FIG. 5. The 2′-OMe modification did not significantly decrease the blunt integration rate at the off-target DSB.

Taken together, these data suggest that the ability of 2′-MOE modifications to reduce homology-independent integration is (a) not a function of increased stability by promoting nuclease resistance as other stabilizing modifications do not result in a similar outcome and (b) not a generalized function of 2′-modifications on the 5′-most ribonucleotide as other 2′-modifications do not result in a similar outcome.

### Example 5

### Use of Hairpins as Blocking Groups to Reduce Homology-Independent Integration.

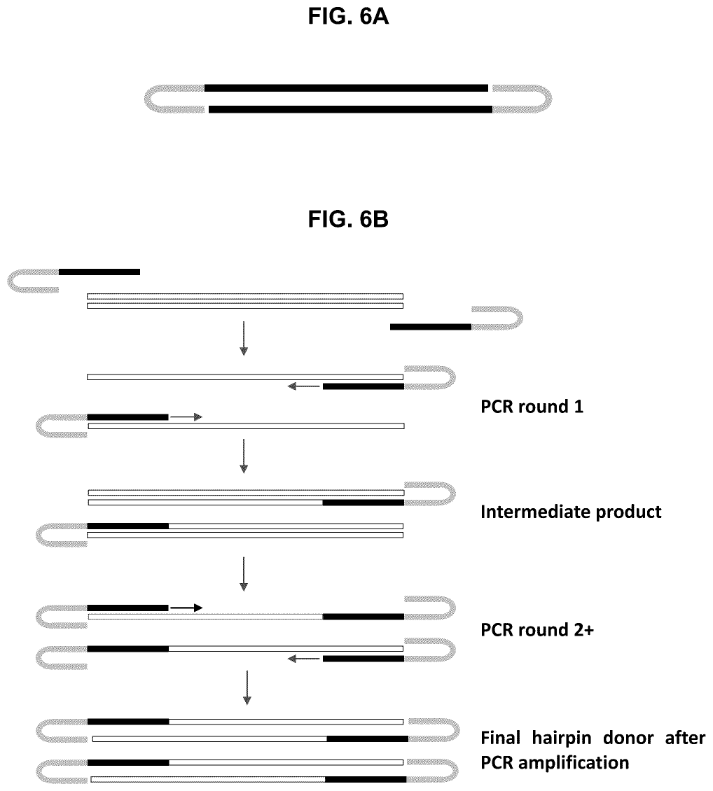

In addition to chemical modifications, the use of DNA hairpins at the ends of dsDNA donors can be used to reduce homology-independent integration. Generation of these hairpin-blocked donors is achieved in several methods (FIG. 6A-B). In the case of small HDR events (generally ≤120 bp insert with 40 bp homology arms), both DNA strands were chemically synthesized with a 5′-MOE hairpin sequence. These MOE adapters contain complementary sequences allowing for the formation of a hairpin structure. The DNA strands were annealed to form a dsDNA HDR donor. In the case of larger HDR events, DNA primers containing a similar 5′-MOE hairpin were chemically synthesized and used for amplification of the desired HDR donor. Use of MOE ribonucleotides within the hairpin structure prevents the procession of the DNA polymerase through the hairpin. For both synthesis methods, hairpin-blocked donors can be used as a nicked HDR template or ligated to generate a fully closed molecule.

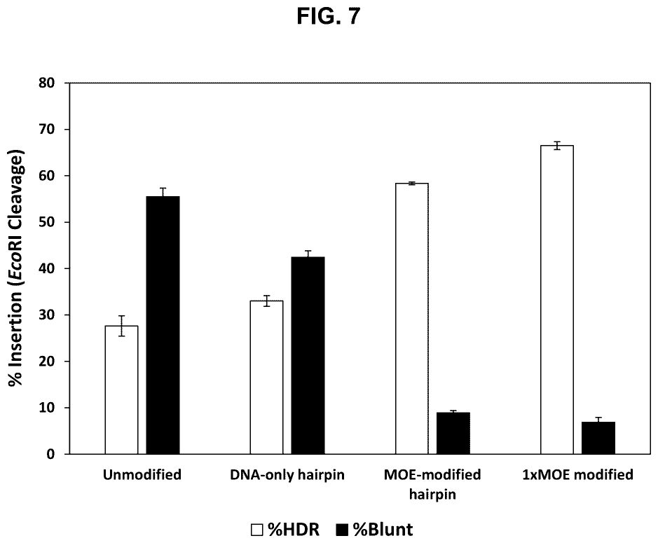

The use of hairpins on chemically synthesized short oligos was functionally tested in cells. A 66-nt sequence was designed to mediate a 6 base GAATTC insertion in the SERPINC 1 locus. This sequence and its reverse complement were synthesized as ssODNs that were either fully unmodified (Table 5; SEQ ID NO: 75-76), contained an unmodified hairpin at the 5′-termini (SEQ ID NO: 77-78), contained a MOE-modified hairpin at the 5′ end (SEQ ID NO: 79-80), or contained a non-template MOE-modified base at the 5′-termini (SEQ ID NO: 81-82). Paired ssODNs were diluted to 100 μM and then mixed at a 1:1 ratio to generate a 50 μM final duplex. The oligo mixtures were heated at 95° C. for 1 min and then slow cooled to room temperature to allow the strands to anneal. Duplexed dsDNA donors were co-delivered with Cas9 complexed with the target gRNA (SEQ ID NO: 22). Donors were delivered at a 2 μM concentration e with 2 μM Cas9 V3 RNP and 2 μM Alt-RR Cas9 Electroporation Enhancer into HEK-293 cells in a final 28 μL volume by Lonza nucleofection (Lonza, Basel Switzerland). DNA was extracted after 48 hrs using QuickExtract™ DNA extraction solution (Lucigen, Madison, WI). Integration rates were assessed by RFLP using EcoRI, with each band quantified on a Fragment Analyzer™ machine (Advanced Analytical, Ames, IA) (FIG. 7).

TABLE 5
Sequences of primers and templates
used in Example 5.

| SEQ ID NO. | Name | Sequence |
|---|---|---|
| SEQ ID NO: 75 | Top Unmod | GATAGGAACTGTAACCT |
| | | CTGGAAAAAGGTAGA |
| | | ATTCAGAGGGGTGAGC |
| | | TTTCCCCTTGCCTGCC |
| | | CC |
| SEQ ID NO: 76 | Bottom Unmod | GGGGCAGGCAAGGGGA |
| | | AAGCTCACCCCTCTGA |
| | | ATTCTACCTTTTTCCA |
| | | GAGGTTACAGTTCCTA |
| | | TC |
| SEQ ID NO: 77 | Top 3DNA HP | pTCGTTTTCGAGATAG |
| | | GAACTGTAACCTCTGG |
| | | AAAAAGGTAGAATTCA |
| | | GAGGGGTGAGCTTTCC |
| | | CCTTGCCTGCCCC |
| SEQ ID NO: 78 | Bottom 3DNA HP | pTCGTTTTCGAGGGGC |
| | | AGGCAAGGGGAAAGCT |
| | | CACCCCTCTGAATTCT |
| | | ACCTTTTTCCAGAGGT |
| | | TACAGTTCCTATC |
| SEQ ID NO: 79 | Top 3MOE HP | pMTMCMGMTMTMTMTM |
| | | CMGMAGATAGGAACTG |
| | | TAACCTCTGGAAAAAG |
| | | GTAGAATTCAGAGGGG |
| | | TGAGCTTTCCCCTTGC |
| | | CTGCCCC |
| SEQ ID NO: 80 | Bottom | pMTMCMGMTMTMTMTM |
| | 3MOE HP | CMGMAGGGGCAGGCAA |
| | | GGGGAAAGCTCACCCC |
| | | TCTGAATTCTACCTTT |
| | | TTCCAGAGGTTACAGT |
| | | TCCTATC |
| SEQ ID NO: 81 | Top 1 × MOE | MAGATAGGAACTGTAA |
| | | CCTCTGGAAAAAGGTA |
| | | GAATTCAGAGGGGTVG |
| | | AGCTTTCCCCTTGCCT |
| | | GCCCC |
| SEQ ID NO: 82 | Bottom | MAGGGGCAGGCAAGGG |
| | 1 × MOE | GAAAGCTCACCCCTCT |
| | | GAATTCTACCTTTTTC |
| | | CAGAGGTTACAGTTCC |
| | | TATC |

DNA is uppercase; p indicates at 5′-phosphate modification; 2′-O-methoxyethyl modified ribonucleotides are shown with an uppercase M preceeding the modified ribonucleotide; non-templated 2′-MOE modified ribonucleotides are shown underlined. Hairpin structures are indicated with italics. All primers and templates were synthesized by IDT (Coralville, IA).

Use of the shorter 66 bp unmodified dsDNA donor resulted in efficient integration through the NHEJ pathway relative to the HDR pathway (55.5% Blunt vs. 27.6% HDR). Introduction of the DNA-only hairpin to the ends of the donor provided an improvement in the repair profile (42.4% Blunt vs. 33.0% HDR). Inclusion of MOE modifications within the hairpin significantly improved this function to similar levels observed with the single 1×MOE on the 5′-termini (58.4% HDR vs. 8.9% Blunt for MOE-modified hairpin; 66.5% HDR vs. 6.9% Blunt for 1×MOE). Additional optimization to the modified hairpins (i.e., ligation, stem-loop length optimization, etc) could be implemented to further improve this function.

### Example 6

### 2′-MOE Modifications Improve Desired Repair Outcomes at Multiple Sites and in Multiple Cell Lines.

In the following experiments, all guides were tested as Alt-R™ crRNA:tracrRNA complexed to Alt-R™ S. pyogenes Cas9 nuclease. RNP complexes and dsDNA donors were delivered to cells of interest using Lonza nucleofection following recommended protocols.

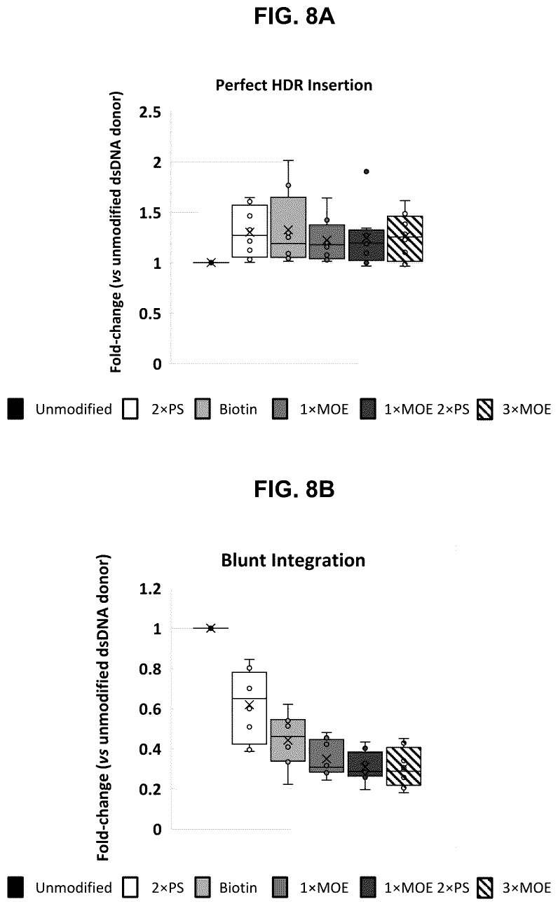

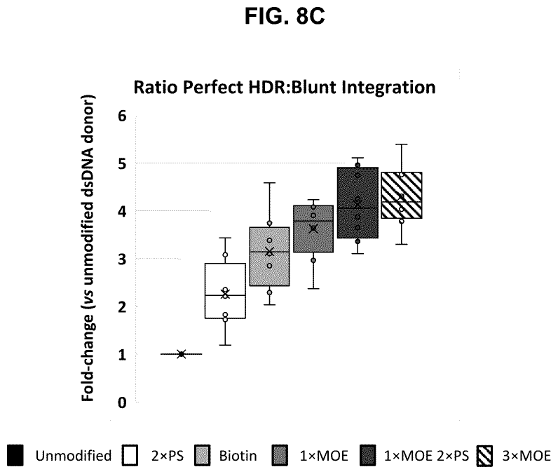

To further validate the ability of the 2′-MOE modification to drive correct repair through higher HDR rates and reduced blunt integration, unmodified and 1×MOE modified dsDNA donors were tested at 4 additional genomic loci (HPRT, AAVS1 670, AAVS1 T2, EMX1) in 2 cell lines (HEK293, K562). Donors were designed to mediate a 42 bp insert and had 40 bp homology arms (SEQ ID NO: 83-86). Donors were generated by PCR amplification as previously described (primer sequences unique to Example 6 listed in Table 6, SEQ ID NO: 95-142). Donors were delivered at 250 nM in a final volume of 28 μL nucleofection buffer with 2 μM Cas9 V3™ RNP (IDT, Coralville, lowa) and 2 μM Alt-R™ Cas9 Electroporation Enhancer™ into the indicated cell lines using recommended protocols for Lonza nucleofection (Lonza, Basel, Switzerland). The protospacer sequences used can be found in Table 6 (SEQ ID NO: 135-138). Cells were lysed after 48 hours using QuickExtract™ DNA extraction solution (Lucigen, Madison, WI). Repair events were quantified by NGS amplicon sequencing (rhAmpSeq™) on the Illumina MiSeq platform (locus specific sequencing primers listed in Table 6, SEQ ID NO: 139-146) and data analysis was performed using IDT's in-house data analysis pipeline (CRISPAltRations), described in U.S. patent application Ser. No. 16/919,577, which is incorporated herein by reference for such teachings (FIG. 8).

TABLE 6
Sequences of primers, crRNAs, and templates
used in Eample 6.

| SEQ | | |
|---|---|---|
| ID | | |
| NO. | Name | Sequence |
| SEQ | HPRT | AGTGCCTTGTCTGTAGTGTCAACTCATTGCTGCCCCT |
| ID | 40-42- | TCCGAATTCTTAGCTCTGTTTACGTCCCAGCGGGCAT |
| NO: | 40 | GAGAGTAATAGTAATCCCCATAATTTAGCTCTCCATT |
| 83 | donor | TCATAGTCTTT |
| SEQ | AAVS1 | AAGGAGGAGGCCTAAGGATGGGGCTTTTCTGTCACCA |
| ID | site1 | ATCGAATTCTTAGCTCTGTTTACGTCCCAGCGGGCAT |
| NO: | 40-42- | GAGAGTAACTGTCCCTAGTGGCCCCACTGTGGGGTGG |
| 84 | 40 | AGGGGACAGAT |

donor

| | | |
|---|---|---|
| SEQ | AAVS1 | TGCCAAGCTCTCCCTCCCAGGATCCTCTCTGGCTCCA |
| ID | site2 | TCGGAATTCTTAGCTCTGTTTACGTCCCAGCGGGCAT |
| NO: | 40-42- | GAGAGTAATAAGCAAACCTTAGAGGTTCTGGCAAGGA |
| 85 | 40 | GAGAGATGGCT |
| | donor | |
| | | AGGCCAATGGGGAGGACATCGATGTCACCTCCAATGA |
| SEQ | EMX1 | CTAGAATTCTTAGCTCTGTTTACGTCCCAGCGGGCAT |
| ID | site | GAGAGTAAGGGTGGGCAACCACAAACCCACGAGGGCA |
| NO: | 40-42- | GAGTGCTGCTT |
| 86 | 40 | |
| | donor | |
| SEQ | HPRT | AGTGCCTTGTCTGTAGTGTCA |
| ID | For | |
| NO: | Unmod | |
| 87 | | |
| SEQ | HPRT | AAAGACTATGAAATGGAGAGCTAAATTATG |
| ID | Rev | |
| NO: | Unmod | |
| 88 | | |
| SEQ | HPRT | A*G*TGCCTTGTCTGTAGTGTCA |
| ID | For | |
| NO: | 2 × PS | |
| 89 | | |
| SEQ | HPRT | A*A*AGACTATGAAATGGAGAGCTAAATTATG |
| ID | Rev | |
| NO: | 2 × PS | |
| 90 | | |
| SEQ | HPRT | B-AGTGCCTTGTCTGTAGTGTCA |
| ID | For | |
| NO: | Biotin | |
| 91 | | |
| SEQ | HPRT | B-AAAGACTATGAAATGGAGAGCTAAATTATG |
| ID | Rev | |
| NO: | Biotin | |
| 92 | | |
| SEQ | HPRT | MAGTGCCTTGTCTGTAGTGTCA |
| ID | For | |
| NO: | 1 × MOE | |
| 93 | | |
| SEQ | HPRT | MAAAGACTATGAAATGGAGAGCTAAATTATG |
| ID | Rev | |
| NO: | 1 × MOE | |
| 94 | | |
| SEQ | HPRT | MA*G*TGCCTTGTCTGTAGTGTCA |
| ID | For | |
| NO: | 1 × MOE | |
| 95 | 2 × PS | |
| SEQ | HPRT | MA*A*AGACTATGAAATGGAGAGCTAAATTATG |
| ID | Rev | |
| NO: | 1 × MOE | |
| 96 | 2 × PS | |
| SEQ | HPRT | MAMGMTGCCTTGTCTGTAGTGTCA |
| ID | For | |
| NO: | 3 × MOE | |
| 97 | | |
| SEQ | HPRT | MAMAMAGACTATGAAATGGAGAGCTAAATTATG |
| ID | Rev | |
| NO: | 3 × MOE | |
| 98 | | |
| SEQ | AAVS1 | AAGGAGGAGGCCTAAGGATGG |
| ID | T2 For | |
| NO: | Unmod | |
| 99 | | |
| SEQ | AAVS1 | ATCTGTCCCCTCCACCCC |
| ID | T2Rev | |
| NO: | Unmod | |
| 100 | | |
| SEQ | AAVS1 | A*A*GGAGGAGGCCTAAGGATGG |
| ID | T2 For | |
| NO: | 2 × PS | |
| 101 | | |
| SEQ | AAVS1 | A*T*CTGTCCCCTCCACCCC |
| ID | T2 Rev | |
| NO: | 2 × PS | |
| 102 | | |
| SEQ | AAVS1 | B-AAGGAGGAGGCCTAAGGATGG |
| ID | T2 For | |
| NO: | Biotin | |
| 103 | | |
| SEQ | AAVS1 | B-ATCTGTCCCCTCCACCCC |
| ID | T2 Rev | |
| NO: | Biotin | |
| 104 | | |
| SEQ | AAVS1 | MAAGGAGGAGGCCTAAGGATGG |
| ID | T2 For | |
| NO: | 1 × MOE | |
| 105 | | |
| SEQ | AAVS1T | MATCTGTCCCCTCCACCCC |
| ID | 2 Rev | |
| NO: | 1 × MOE | |
| 106 | | |
| SEQ | AAVS1 | MA*A*GGAGGAGGCCTAAGGATGG |
| ID | T2 For | |
| NO: | 1 × MOE | |
| 107 | 2 × PS | |
| SEQ | AAVS1 | MA*T*CTGTCCCCTCCACCCC |
| ID | T2 Rev | |
| NO: | 1 × MOE | |
| 108 | 2 × PS | |
| SEQ | AAVS1 | MAMAMGGAGGAGGCCTAAGGATGG |
| ID | T2 For | |
| NO: | 3 × MOE | |
| 109 | | |
| SEQ | AAVS1 | MAMTMCTGTCCCCTCCACCCC |
| ID | T2 Rev | |
| NO: | 3 × MOE | |
| 110 | | |
| SEQ | AAVS1 | TGCCAAGCTCTCCCTCCC |
| ID | 670 For | |
| NO: | Unmod | |
| 111 | | |
| SEQ | AAVS1 | AGCCATCTCTCTCCTTGCCAG |
| ID | 670 Rev | |
| NO: | Unmod | |
| 112 | | |
| SEQ | AAVS1 | T*G*CCAAGCTCTCCCTCCC |
| ID | 670 For | |
| NO: | 2 × PS | |
| 113 | | |
| SEQ | AAVS1 | A*G*CCATCTCTCTCCTTGCCAG |
| ID | 670 Rev | |
| NO: | 2 × PS | |
| 114 | | |
| SEQ | AAVS1 | B-TGCCAAGCTCTCCCTCCC |
| ID | 670 For | |
| NO: | Biotin | |
| 115 | | |
| SEQ | AAVS1 | B-AGCCATCTCTCTCCTTGCCAG |
| ID | 670 Rev | |
| NO: | Biotin | |
| 116 | | |
| SEQ | AAVS1 | MTGCCAAGCTCTCCCTCCC |
| ID | 670 For | |
| NO: | 1 × MOE | |
| 117 | | |
| SEQ | AAVS1 | MAGCCATCTCTCTCCTTGCCAG |
| ID | 670 Rev | |
| NO: | 1 × MOE | |
| 118 | | |
| SEQ | AAVS1 | MT*G*CCAAGCTCTCCCTCCC |
| ID | 670 For | |
| NO: | 1 × MOE | |
| 119 | 2 × PS | |
| SEQ | AAVS1 | MA*G*CCATCTCTCTCCTTGCCAG |
| ID | 670 Rev | |
| NO: | 1 × MOE | |
| 120 | 2 × PS | |
| SEQ | AAVS1 | MTMGMCCAAGCTCTCCCTCCC |
| ID | 670 For | |
| NO: | 3 × MOE | |
| 121 | | |
| SEQ | AAVS1 | MAMGMCCATCTCTCTCCTTGCCAG |
| ID | 670 Rev | |
| NO: | 3 × MOE | |
| SEQ | EMX1 | AGGCCAATGGGGAGGACATC |
| ID | For | |
| NO: | Unmod | |
| 123 | | |
| SEQ | EMX1 | AAGCAGCACTCTGCCCTCG |
| ID | Rev | |
| NO: | Unmod | |
| 124 | | |
| SEQ | EMX1 | A*G*GCCAATGGGGAGGACATC |
| ID | For | |
| NO: | 2 × PS | |
| 125 | | |
| SEQ | EMX1 | A*A*GCAGCACTCTGCCCTCG |
| ID | Rev | |
| NO: | 2 × PS | |
| 126 | | |
| SEQ | EMX1 | B-AGGCCAATGGGGAGGACATC |
| ID | For | |
| NO: | Biotin | |
| 127 | | |
| SEQ | EMX1 | B-AAGCAGCACTCTGCCCTCG |
| ID | Rev | |
| NO: | Biotin | |
| 128 | | |
| SEQ | EMX1 | MAGGCCAATGGGGAGGACATC |
| ID | For | |
| NO: | 1 × MOE | |
| 129 | | |
| SEQ | EMX1 | MAAGCAGCACTCTGCCCTCG |
| ID | Rev1 | |
| NO: | × MOE | |
| 130 | | |
| SEQ | EMX1 | MA*G*GCCAATGGGGAGGACATC |
| ID | For | |
| NO: | 1 × MOE | |
| 131 | 2 × PS | |
| SEQ | EMX1 | MA*A*GCAGCACTCTGCCCTCG |
| ID | Rev | |
| NO: | 1 × MOE | |
| 132 | 2 × PS | |
| SEQ | EMX1 | MAMGMGCCAATGGGGAGGACATC |
| ID | For | |
| NO: | 3 × MOE | |
| 133 | | |
| SEQ | EMX1 | MAMAMGCAGCACTCTGCCCTCG |
| ID | Rev | |
| NO: | 3 × MOE | |
| 134 | | |
| SEQ | HPRT | AATTATGGGGATTACTAGGA |
| ID | gRNA | |
| NO: | proto- | |
| 135 | spacer | |
| SEQ | AAVS1 | GGGGCCACTAGGGACAGGAT |
| ID | T2 | |
| NO: | gRNA | |
| 136 | proto- | |
| | spacer | |
| SEQ | AAVS1 | CCTCTAAGGTTTGCTTACGA |
| ID | 670 | |
| NO: | gRNA | |
| 137 | proto- | |
| | spacer | |
| SEQ | EMX1 | GTCACCTCCAATGACTAGGG |
| ID | gRNA | |
| NO: | proto- | |
| 138 | spacer | |
| SEQ | HPRT | ACACTCTTTCCCTACACGACGCTCTTCCGA |
| ID | NGS | TCTCAGAACTGTCCTTCAGGTTC |
| NO: | For | |
| 139 | | |
| SEQ | HPRT | GTGACTGGAGTTCAGACGTGTGCTCTTCCG |
| ID | NGS | ATCTCACTGTTTCATTTCATCCGTG |
| NO: | Rev | |
| 140 | | |
| SEQ | AAVS1 | ACACTCTTTCCCTACACGACGCTCTTCCGA |
| ID | T2 | TCTGAGAGATGGCTCCAGGAAATG |
| NO: | NGS | |
| 141 | For | |
| SEQ | AAVS1 | GTGACTGGAGTTCAGACGTGTGCTCTTCCG |
| ID | T2 | ATCTCACTTCAGGACAGCATGTTTG |
| NO: | NGS | |
| 142 | Rev | |
| SEQ | AAVS1 | ACACTCTTTCCCTACACGACGCTCTTCCGA |
| ID | 670 | TCTGATCAGTGAAACGCACCAGA |
| NO: | NGS | |
| 143 | For | |
| SEQ | AAVS1 | GTGACTGGAGTTCAGACGTGTGCTCTTCCG |
| ID | 670 | ATCTCCTCCTTCCTAGTCTCCTGATATT |
| NO: | NGS | |
| 144 | Rev | |
| SEQ | EMX1 | ACACTCTTTCCCTACACGACGCTCTTCCGA |
| ID | NGS | TCTAGAAGAAGAAGGGCTCCCA |
| NO: | For | |
| 145 | | |
| SEQ | EMX1 | GTGACTGGAGTTCAGACGTGTGCTCTTCCG |
| ID | NGS | ATCTCAGGGAGTGGCCAGAGT |
| NO: | Rev | |
| 146 | | |

DNA is uppercase; RNA is lowercase; B- is a 5′-biotin moiety; phosphorothioate (PS) modified linkages are shown with an asterisk (*); and 2′-O-methoxyethyl modified ribonucleotides are shown with an uppercase M preceeding the modified ribonucleotide. All primers and templates were synthesized by IDT (Coralville, IA).

As previously observed, the 5′-modifications all improved HDR rates over unmodified dsDNA donors (A). While the fold-improvement in HDR over an unmodified dsDNA varied across sites and cell lines, the average improvement in HDR rates were relatively similar for all modifications tested (ranging from a 1.2 to 1.3-fold improvement). In contrast, MOE modified donors displayed a greater reduction in blunt integration compared to the 2×PS and Biotin donors (B). On average, the fold reductions in blunt integration relative to unmodified dsDNA were 1.6 (2×PS), 2.3 (Biotin), 2.9 (1×MOE), 3.2 (1×MOE, 2×PS), and 3.3 (3×MOE). When the improvements in HDR and blunt integration were assessed together for each site (C, reported as the ratio of HDR:Blunt repair events), MOE modified dsDNA donors outperformed other modifications. The average fold change over unmodified dsDNA were 2.3 (2×PS), 3.1 (Biotin), 3.6 (1×MOE), 4.1 (1×MOE 2×PS), and 4.3 (3×MOE). Taken together, these results demonstrate that MOE modifications are the most efficient at driving the correct repair event following CRISPR editing.

### Example 7

HDR Rates Are Increased, and NHEJ Insertions Are Reduced With Modified dsDNA Donors Mediating Large Insertions.

As a follow-up to the work with short insertions, experiments were performed to compare the HDR and blunt integration rates when using dsDNA donors mediating 300 bp, 500 bp, or 1000 bp insertions at two genomic loci (SERPINC1 and EMX1; see Table 7 for donor sequences and amplification primers, SEQ ID NO: 147-154). Donors were generated by PCR amplification of plasmids containing the desired inserts with 100 bp of flanking homology arms. Amplification primers were designed with either unmodified sequence or the indicated modifications. Long ssDNA (Megamers™) were ordered for comparison at the SERPINC1 locus. Donors were delivered at 100 nM in a final volume of 28 μL nucleofection buffer with 2 μM Cas9 V3™ RNP (IDT, Coralville, lowa) targeting SERPINC1 or EMX1 into 3.5×10⁵ HEK-293 cells using Lonza nucleofection (Lonza, Basel, Switzerland). Cells were treated with the IDT Alt-R™ HDR Enhancer V2 (1 μM) for 24 hrs post-transfection. The protospacer sequences used is shown in Table 1 (SEQ ID NO: 22) and Table 7 (SEQ ID NO: 161).

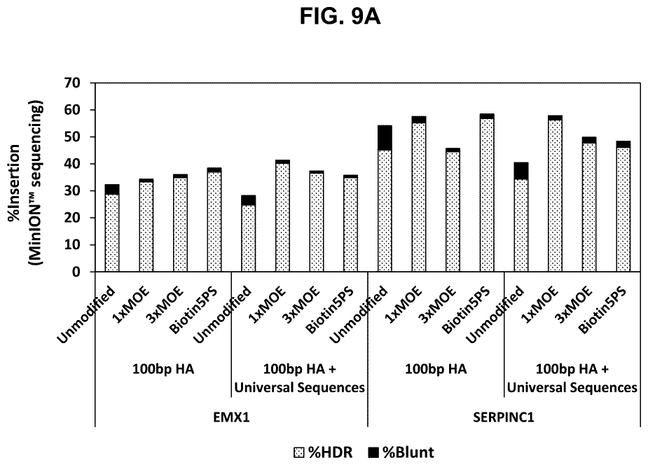

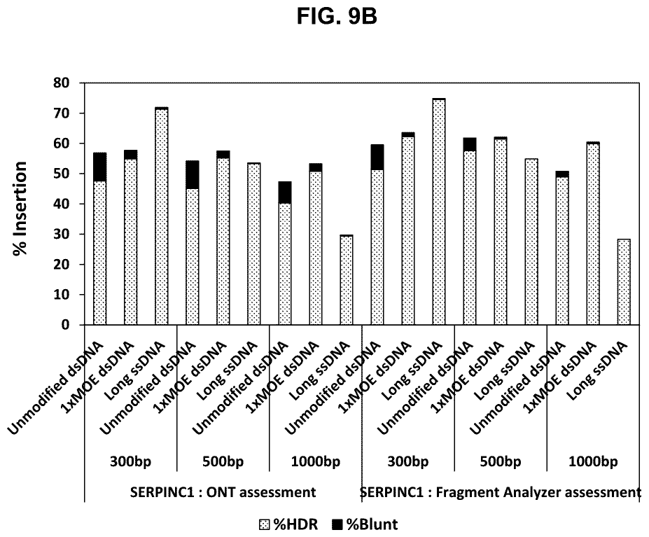

Cells were lysed after 48 hours using QuickExtract™ DNA extraction solution (Lucigen, Madison, WI). HDR and blunt integration rates were assessed by long-read sequencing using the MinION™ platform (Oxford Nanopore Technologies, Oxford, UK). Locus specific amplification primers used are listed (Table 7, SEQ ID NO: 162-165). Final sequencing libraries were prepared with the PCR Barcoding Expansion and Ligation Sequencing Kit following the manufacturer's recommended protocols. Final data analysis was performed using IDT's in-house data analysis pipeline (CRISPAltRations) (FIG. 9A). Insertion rates were independently assessed by amplicon length analysis for the SERPINC1 samples. Isolated gDNA was PCR amplified using the SERPINC1 RFLP primers (SEQ ID NO: 45-46). Amplicons were run on a Fragment Analyzer™ machine for band quantification. Insertion events were identified based on expected amplicon sizes for integration events (FIG. 9B).

TABLE 7
Sequences of Primers and Templates used in Example 7. SEQ ID NO: 22 used for
SERPINC1 crRNA (Table 1).

| SEQ ID NO. | Name | Sequence |
|---|---|---|
| SEQ ID NO: 147 | SC1 100-300-100 | GATTGCCTCAGATCACACTATCTCCACTTGCCCAGCCCTGT |
| | donor | GGAAGATTAGCGGCCATGTATTCCAATGTGATAGGAACTGT |
| | | AACCTCTGGAAAAAGGTACGAATTCGAGGGCAGAGGCAGTC |
| | | TGCTGACATGCGGTGACGTGGAAGAGAATCCCGGCCCTTCT |
| | | AGATAACTGTGCCTTCTAGTTGCCAGCCATCTGTTGTTTGC |
| | | CCCTCCCCCGTGCCTTCCTTGACCCTGGAAGGTGCCACTCC |
| | | CACTGTCCTTTCCTAATAAAATGAGGAAATTGCATCGCATT |
| | | GTCTGAGTAGGTGTCATTCTATTCTGGGGGGTGGGGTGGGG |
| | | CAGGACAGCAAGGGGGAGGATTGGGAAGACAATAGCAGGCA |
| | | TGCTGGGGATGCGGTGGGCTCTATGGCGGTACCAGAGGGGT |
| | | GAGCTTTCCCCTTGCCTGCCCCTACTGGGTTTTGTGACCTC |
| | | CAAAGGACTCACAGGAATGACCTCCAACACCTTTGAGAAGA |
| | | CCAGGCCCTC |
| SEQ ID NO: 148 | SC1 100-500-100 | GATTGCCTCAGATCACACTATCTCCACTTGCCCAGCCCTGT |
| | donor | GGAAGATTAGCGGCCATGTATTCCAATGTGATAGGAACTGT |
| | | AACCTCTGGAAAAAGGTACGAATTCGAGGGCAGAGGCAGTC |
| | | TGCTGACATGCGGTGACGTGGAAGAGAATCCCGGCCCTTCT |
| | | AGAATGGTTAGCAAGGGCGAGGAGCTGTTCACCGGGGTGGT |
| | | GCCCATCCTGGTCGAGCTGGACGGCGACGTAAACGGCCACA |
| | | AGTTCAGCGTGTCCGGCGAGGGCGAGGGCGATGCCACCTAC |
| | | GGCAAGCTGACCCTGAAGTTCATCTGCACCACCGGCAAGCT |
| | | GCCCGTGCCCTGGCCCACCCTCGTGACCACCCTGACCTAAC |
| | | TGTGCCTTCTAGTTGCCAGCCATCTGTTGTTTGCCCCTCCC |
| | | CCGTGCCTTCCTTGACCCTGGAAGGTGCCACTCCCACTGTC |
| | | CTTTCCTAATAAAATGAGGAAATTGCATCGCATTGTCTGAG |
| | | TAGGTGTCATTCTATTCTGGGGGGTGGGGTGGGGCAGGACA |
| | | GCAAGGGGGAGGATTGGGAAGACAATAGCAGGCATGCTGGG |
| | | GATGCGGTGGGCTCTATGGCGGTACCAGAGGGGTGAGCTTT |
| | | CCCCTTGCCTGCCCCTACTGGGTTTTGTGACCTCCAAAGGA |
| | | CTCACAGGAATGACCTCCAACACCTTTGAGAAGACCAGGCC |
| | | CTC |
| SEQ ID NO: 1 | SC1 100-1000-100 | GATTGCCTCAGATCACACTATCTCCACTTGCCCAGCCCTGT |
| SEQ ID NO: 149 | donor2 | GGAAGATTAGCGGCCATGTATTCCAATGTGATAGGAACTGT |
| | | AACCTCTGGAAAAAGGTACGAATTCGAGGGCAGAGGCAGTC |
| | | TGCTGACATGCGGTGACGTGGAAGAGAATCCCGGCCCTTCT |
| | | AGAATGGTTAGCAAGGGCGAGGAGCTGTTCACCGGGGTGGT |
| | | GCCCATCCTGGTCGAGCTGGACGGCGACGTAAACGGCCACA |
| | | AGTTCAGCGTGTCCGGCGAGGGCGAGGGCGATGCCACCTAC |
| | | GGCAAGCTGACCCTGAAGTTCATCTGCACCACCGGCAAGCT |
| | | GCCCGTGCCCTGGCCCACCCTCGTGACCACCCTGACCTACG |
| | | GCGTGCAGTGCTTCAGCCGCTACCCCGACCACATGAAGCAG |
| | | CACGACTTCTTCAAGTCCGCCATGCCCGAAGGCTACGTCCA |
| | | GGAGCGCACCATCTTCTTCAAGGACGACGGCAACTACAAGA |
| | | CCCGCGCCGAGGTGAAGTTCGAGGGCGACACCCTGGTGAAC |
| | | CGCATCGAGCTGAAGGGCATCGACTTCAAGGAGGACGGCAA |
| | | CATCCTGGGGCACAAGCTTGAGTACAACTACAACAGCCACA |
| | | ACGTCTATATCATGGCCGACAAGCAGAAGAACGGCATCAAG |
| | | GTGAACTTCAAGATCCGCCACAACATCGAGGACGGCAGCGT |
| | | GCAGCTCGCCGACCACTACCAGCAGAACACCCCCATCGGCG |
| | | ACGGCCCCGTGCTGCTGCCCGACAACCACTACCTGAGCACC |
| | | CAGTCCGCCCTGAGCAAAGACCCCAACGAGAAGCGCGATCA |
| | | CATGGTCCTGCTGGAGTTCGTGACCGCCGCCGGGATCACTC |
| | | TCGGCATGGACGAGCTGTACAAGTAACTGTGCCTTCTAGTT |
| | | GCCAGCCATCTGTTGTTTGCCCCTCCCCCGTGCCTTCCTTG |
| | | ACCCTGGAAGGTGCCACTCCCACTGTCCTTTCCTAATAAAA |
| | | TGAGGAAATTGCATCGCATTGTCTGAGTAGGTGTCATTCTA |
| | | TTCTGGGGGGTGGGGTGGGGCAGGACAGCAAGGGGGAGGAT |
| | | TGGGAAGACAATAGCAGGCATGCTGGGGATGCGGTGGGCTC |
| | | TATGGCGGTACCAGAGGGGTGAGCTTTCCCCTTGCCTGCCC |
| | | CTACTGGGTTTTGTGACCTCCAAAGGACTCACAGGAATGAC |
| | | CTCCAACACCTTTGAGAAGACCAGGCCCTC |
| SEQ ID NO: 150 | EMX 1 100-300-100 | CTCCCTCCCTGGCCCAGGTGAAGGTGTGGTTCCAGAACCGG |
| | donor | AGGACAAAGTACAAACGGCAGAAGCTGGAGGAGGAAGGGCC |
| | | TGAGTCCGAGCAGAAGAACGAATTCGAGGGCAGAGGCAGTC |
| | | TGCTGACATGCGGTGACGTGGAAGAGAATCCCGGCCCTTCT |
| | | AGATAACTGTGCCTTCTAGTTGCCAGCCATCTGTTGTTTGC |
| | | GCCTCACTCGTGCCTTCATTGACCCTGGAAGGTGCCACTCC |
| | | CACTGTCCTTTCCTAATAAAATGAGGAAATTGCATCGCATT |
| | | GTCTGAGTAGGTGTCATTCTATTCTGGCGTATCGAGTGGCT |
| | | CAGGACAGCAAGAGCGAGGATTGGGAAGACAATAGCAGGCA |
| | | TGCTGGGGATGCGGTGGGCTCTATGGCGGTACCGAAGGGCT |
| | | CCCATCACATCAACCGGTGGCGCATTGCCACGAAGCAGGCC |
| | | AATGGGGAGGACATCGATGTCACCTCCAATGACTAGGGTGG |
| | | GCAACCACAA |
| SEQ ID NO: 151 | EMX1 100-500-100 | CTCCCTCCCTGGCCCAGGTGAAGGTGTGGTTCCAGAACCGG |
| | donor | AGGACAAAGTACAAACGGCAGAAGCTGGAGGAGGAAGGGCC |
| | | TGAGTCCGAGCAGAAGAACGAATTCGAGGGCAGAGGCAGTC |
| | | TGCTGACATGCGGTGACGTGGAAGAGAATCCCGGCCCTTCT |
| | | AGAATGGTTAGCAAGGGCGAGGAGCTGTTCACCGGGGTGGT |
| | | GCCCATCCTGGTCGAGCTGGACGGCGACGTAAACGGCCACA |
| | | AGTTCAGCGTGTCCGGCGAGGGAGAGGGCGATGCCACCTAC |
| | | GGCAAGCTGACCCTGAAGTTCATCTGCACCACCGGCAAGCT |
| | | GCCAGTGCCCTGGCCTACCCTCGTGACCACCCTGACCTAAC |
| | | TGTGCCTTCTAGTTGCCAGCCATCTGTTGTTTGCGCCTCAC |
| | | TCGTGCCTTCATTGACCCTGGAAGGTGCCACTCCCACTGTC |
| | | CTTTCCTAATAAAATGAGGAAATTGCATCGCATTGTCTGAG |
| | | TAGGTGTCATTCTATTCTGGCGTATCGAGTGGCTCAGGACA |
| | | GCAAGAGCGAGGATTGGGAAGACAATAGCAGGCATGCTGGG |
| | | GATGCGGTGGGCTCTATGGCGGTACCGAAGGGCTCCCATCA |
| | | CATCAACCGGTGGCGCATTGCCACGAAGCAGGCCAATGGGG |
| | | AGGACATCGATGTCACCTCCAATGACTAGGGTGGGCAACCA |
| | | CAA |
| SEQ ID NO: 152 | EMX1 100-1000-100 | CTCCCTCCCTGGCCCAGGTGAAGGTGTGGTTCCAGAACCGG |
| | donor | AGGACAAAGTACAAACGGCAGAAGCTGGAGGAGGAAGGGCC |
| | | TGAGTCCGAGCAGAAGAACGAATTCGAGGGCAGAGGCAGTC |
| | | TGCTGACATGCGGTGACGTGGAAGAGAATCCCGGCCCTTCT |
| | | AGAATGGTTAGCAAGGGCGAGGAGCTGTTCACCGGGGTGGT |
| | | GCCCATCCTGGTCGAGCTGGACGGCGACGTAAACGGCCACA |
| | | AGTTCAGCGTGTCCGGCGAGGGAGAGGGCGATGCCACCTAC |
| | | GGCAAGCTGACCCTGAAGTTCATCTGCACCACCGGCAAGCT |
| | | GCCAGTGCCCTGGCCTACCCTCGTGACCACCCTGACCTACG |
| | | GCGTGCAGTGCTTCAGCCGCTACCCCGACCACATGAAGCAG |
| | | CACGACTTCTTCAAGTCCGCCATGCCCGAAGGCTACGTCCA |
| | | GGAGCGCACCATCTTCTTCAAGGACGACGGCAACTACAAGA |
| | | CCCGTGCCGAGGTGAAGTTCGAAGGCGACACCCTGGTGAAC |
| | | CGCATCGAGCTGAAGGGCATCGACTTCAAGGAGGACGGCAA |
| | | CATCCTGGGGCACAAGCTTGAGTACAACTACAACAGCCACA |
| | | ACGTCTATATCATGGCCGACAAGCAGAAGAACGGCATCAAG |
| | | GTGAACTTCAAGATCCGCCACAACATCGAGGACGGTAGCGT |
| | | GCAGCTCGCTGACCACTACCAGCAGAACACTCCTATCGGAG |
| | | ACGGTCCTGTGCTGCTGCCAGACAACCACTACCTGAGCACA |
| | | CAGTCCGCTCTGAGCAAAGACCCTAACGAGAAGCGCGATCA |
| | | CATGGTCCTGCTGGAGTTCGTGACAGCCGCTGGGATCACTC |
| | | TCGGCATGGACGAGCTGTACAAGTAACTGTGCCTTCTAGTT |
| | | GCCAGCCATCTGTTGTTTGCGCCTCACTCGTGCCTTCATTG |
| | | ACCCTGGAAGGTGCCACTCCCACTGTCCTTTCCTAATAAAA |
| | | TGAGGAAATTGCATCGCATTGTCTGAGTAGGTGTCATTCTA |
| | | TTCTGGCGTATCGAGTGGCTCAGGACAGCAAGAGCGAGGAT |
| | | TGGGAAGACAATAGCAGGCATGCTGGGGATGCGGTGGGCTC |
| | | TATGGCGGTACCGAAGGGCTCCCATCACATCAACCGGTGGC |
| | | GCATTGCCACGAAGCAGGCCAATGGGGAGGACATCGATGTC |
| | | ACCTCCAATGACTAGGGTGGGCAACCACAA |
| SEQ ID NO: 2 | SC1 100 Fwd unmod2 | GATTGCCTCAGATCACACTATCTCCACTTGCC |
| SEQ ID NO: 153 | | |
| SEQ ID NO: 3 | SC1 100 Rev unmod2 | GAGGGCCTGGTCTTCTCAAAGGTGTTG |
| SEQ ID NO: 154 | | |
| SEQ ID NO: 20 | SC1 100 Fwd MOE2 | MGATTGCCTCAGATCACACTATCTCCACTTGCC |
| SEQ ID NO: 155 | | |
| SEQ ID NO: 21 | SC1 100 Rev MOE2 | MGAGGGCCTGGTCTTCTCAAAGGTGTTG |
| SEQ ID NO: 156 | | |
| SEQ ID NO: 157 | EMX1 100 Fwd unmod | CTCCCTCCCTGGCCCAGGTGAAG |
| SEQ ID NO: 158 | EMX1 100 Rev unmod | TTGTGGTTGCCCACCCTAGTCATTGGA |
| SEQ ID NO: 159 | EMX1 100 Fwd MOE | MCTCCCTCCCTGGCCCAGGTGAAG |
| SEQ ID NO: 160 | EMX1 100 Rev MOE | MTTGTGGTTGCCCACCCTAGTCATTGGA |
| SEQ ID NO: 161 | EMX1 guide | GAGTCCGAGCAGAAGAAGAA |
| | protospacer | |
| SEQ ID NO: 162 | SC1 ONT For | TTTCTGTTGGTGCTGATATTGCCTTTATGTGATTGCTGTAT |
| | | GTCTCC |
| SEQ ID NO: 163 | SC1 ONT Rev | ACTTGCCTGTCGCTCTATCTTCGAATCTGCCAGGTGCTGAT |
| | | A |
| SEQ ID NO: 164 | EMX1 ONT For | TTTCTGTTGGTGCTGATATTGCCTGTGCTTTACCCAGTTCT |
| | | CT |
| SEQ ID NO: 165 | EMX1 ONT Rev | ACTTGCCTGTCGCTCTATCTTCGCTGGGTCTCTGACATCTT |
| | | T |

DNA is uppercase; and 2′-O-methoxyethyl modified ribonucleotides are shown with an uppercase M preceeding the modified ribonucleotide. SC1 is SERPINC1. All primers and templates were synthesized by IDT (Coralville, IA).

1×MOE modified donors resulted in higher HDR rates (on average from 28.6% to 30.9% at EMX1 and 44.4% to 53.6% at SERPINC1) and lower blunt integration rates (on average from 3.2% to 1.1% at EMX1 and 8.4% to 2.5% at SERPINC1) when compared to unmodified dsDNA donors mediating large insertions at both genomic loci. Long ssDNA donors mediating the same insertions at the SERPINC1 locus resulted in extremely low blunt integration rates (<1%). The long ssDNA donor mediated the highest rates of HDR for the 300 bp insert (71.3% vs. 54.9% with a modified dsDNA donor). However, the modified dsDNA donor performed as well or better than the long ssDNA for HDR with the larger insertions (55.2% vs. 53.3% for a 500 bp insert, 50.8% vs. 29.4% for a 1000 bp insert). Similar trends were observed for the SERPINC1 samples when an orthologous method of assessment was used (FIG. 9B).

### Example 8

Utilization of Universal Priming Sequences for Manufacturing Modified dsDNA Donors Does Not Adversely Affect HDR Repair.

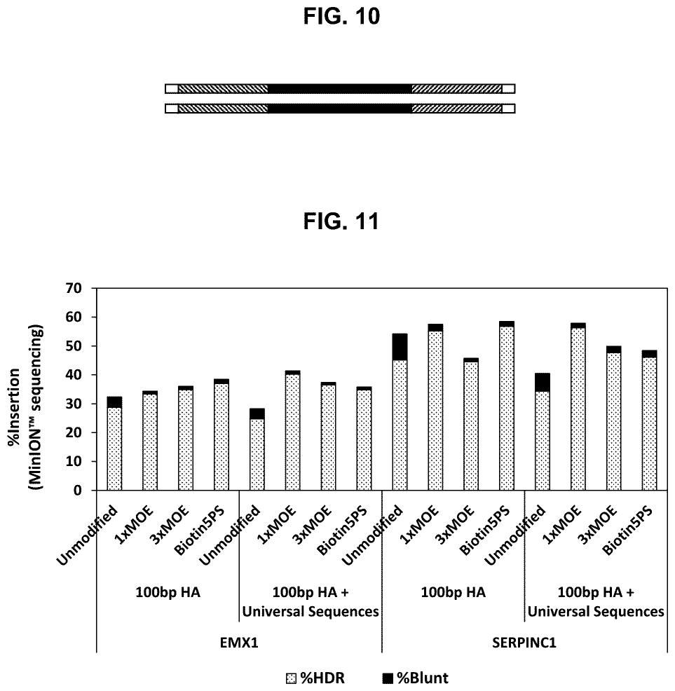

To assess the impact of incorporating universal priming sequences into the donor template, dsDNA donors mediating a 500 bp insert at EMX1 and SERPINC1 (see Table 7, SEQ ID NO: 148 and 151) were prepared with either locus specific primers or with universal primers (Table 7 SEQ ID NO: 153-160; Table 8 SEQ ID NO: 166-181). Placement of the universal priming sequences relative to the donor is shown in FIG. 10. Modifications tested included 1×MOE, 3×MOE, and Biotin with phosphorothioate modifications (Biotin5×PS, as described in [5]). Donors were delivered at 100 nM in a final volume of 28 μL nucleofection buffer with 2 μM Cas9 V3™ RNP (IDT, Coralville, lowa) targeting SERPINC1 or EMX1 into 3.5×10⁵ HEK-293 cells using Lonza nucleofection (Lonza, Basel, Switzerland). Cells were treated with the IDT Alt-R™M HDR Enhancer V2 (1 μM) for 24 hrs post-transfection. Cells were lysed after 48 hours using QuickExtract™ DNA extraction solution (Lucigen, Madison, WI). HDR and blunt integration rates were assessed by long-read sequencing using the MinION™ platform (Oxford Nanopore Technologies, Oxford, UK) and analyzed as previously described (FIG. 11).

TABLE 8
Sequences of Primers for dsDNA
Donor Synthesis in Example 8

| SEQ ID NO. | Name | Sequence |
|---|---|---|
| SEQ ID NO: 166 | SC1 100 Fwd | MGMAMTTGCC |
| | 3 × MOE | TCAGATCACA |
| | | CTATCTCCAC |
| | | TTGCC |
| SEQ ID NO: 167 | SC1 100 Rev | MGMAMGGGCC |
| | 3 × MOE | TGGTCTTCTC |
| | | AAAGGTGTTG |
| SEQ ID NO: 168 | SC1 100 Fwd | B-G*A*T*T* |
| | Biotin5PS | G*CCTCAGAT |
| | | CACACTATCT |
| | | CCACTTGCC |
| SEQ ID NO: 169 | SC1 100 Rev | B-G*A*G*G* |
| | Biotin5 × PS | G*CCTGGTCT |
| | | TCTCAAAGGT |
| | | GTTG |
| SEQ ID NO: 170 | EMX1 100 Fwd | MCMTMCCCTC |
| | 3 × MOE | CCTGGCCCAG |
| | | GTGAAG |
| SEQ ID NO: 171 | EMX1 100 Rev | MTMTMGTGGT |
| | 3 × MOE | TGCCCACCCT |
| | | AGTCATTGGA |
| SEQ ID NO: 172 | EMX1 100 Fwd | B-C*T*C*C* |
| | Biotin5 × PS | C*TCCCTGGC |
| | | CCAGGTGAAG |
| SEQ ID NO: 173 | EMX1 100 Rev | B-T*T*G*T* |
| | Biotin5 × PS | G*GTTGCCCA |
| | | CCCTAGTCAT |
| | | TGGA |
| SEQ ID NO: 174 | Universal For | GTCGTACCGA |
| | unmod | CTGGTAGATG |
| | | ACAGCAAACC |
| SEQ ID NO: 175 | Universal Rev | GGTCTCGACT |
| | unmod | ATACGCCCGT |
| | | TTTCGGATC |
| SEQ ID NO: 176 | Universal | MGTCGTACCG |
| | For 1 × MOE | ACTGGTAGAT |
| | | GACAGCAAAC |
| | | C |
| SEQ ID NO: 177 | Universal Rev | MGGTCTCGAC |
| | 1 × MOE | TATACGCCCG |
| | | TTTTCGGATC |
| SEQ ID NO: 178 | Universal For | MGMTMCGTAC |
| | 3 × MOE | CGACTGGTAG |
| | | ATGACAGCAA |
| | | ACC |
| SEQ ID NO: 179 | Universal Rev | MGMGMTCTCG |
| | 3 × MOE | ACTATACGCC |
| | | CGTTTTCGGA |
| | | TC |
| SEQ ID NO: 180 | Universal For | B-G*T*C*G* |
| | Biotin5 × PS | T*ACCGACTG |
| | | GTAGATGACA |
| | | GCAAACC |
| SEQ ID NO: 181 | Universal Rev | B-G*G*T*C* |
| | Biotin5 × PS | T*CGACTATA |
| | | CGCCCGTTTT |
| | | CGGATC |

DNA is uppercase; 2′-O-methoxyethyl modified ribonucleotides are shown with an uppercase M preceeding the modified ribonucleotide; B- is a 5′-biotin moiety; and phosphorothioate (PS) modified linkages are shown with an asterisk (*). SC1 is SERPINC1. All primers and templates were synthesized by IDT (Coralville, IA).

HDR and blunt integration rates were relatively similar for dsDNA donors generated with or without the universal priming sequences. For donors without universal priming sequences, the improvement to HDR and reduction in blunt rates were similar across the various modifications. The major exception to this trend was the 3×MOE modification for the SERPINC1 site, where blunt insertion was still reduced relative to an unmodified dsDNA but HDR was not improved to the same extent as 1×MOE or Biotin5×PS (unmod: 45.1% HDR, 9.0% Blunt; 1×MOE: 55.2% HDR, 2.3% Blunt, 3×MOE: 44.5% HDR, 1.2% Blunt). In contrast, a much larger difference in performance was observed with the modified donors manufactured with the universal priming sequences. Interestingly, the unmodified dsDNA donors for both sites had lower integration rates when universal priming sites were included in the donor sequence (EMX1: 28.8% vs 24.8% HDR, SERPINC 1: 45.2% vs 34.4% HDR). For both sites, the 1×MOE modification offered the greatest improvement to the HDR rate when incorporated into donors containing the universal sequences.

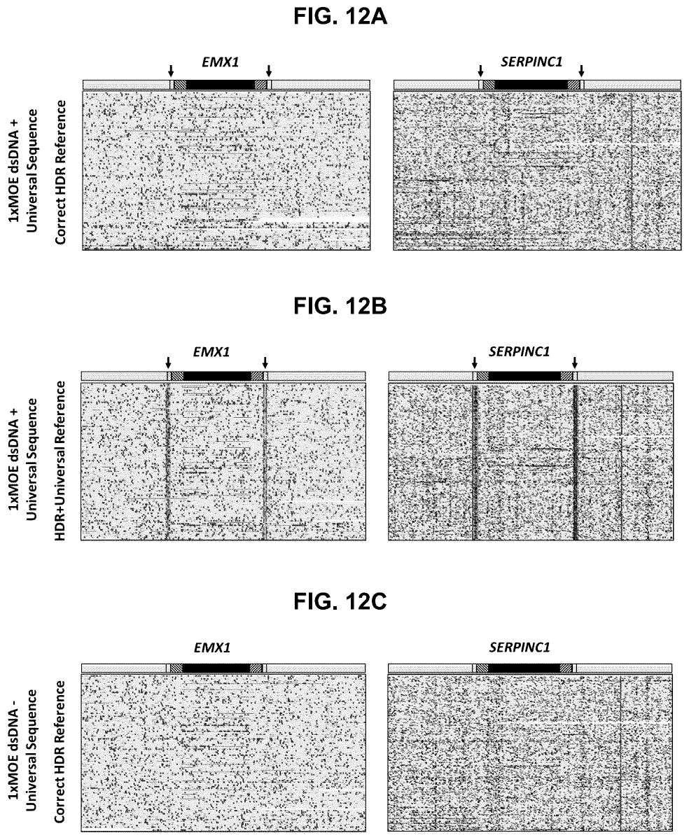

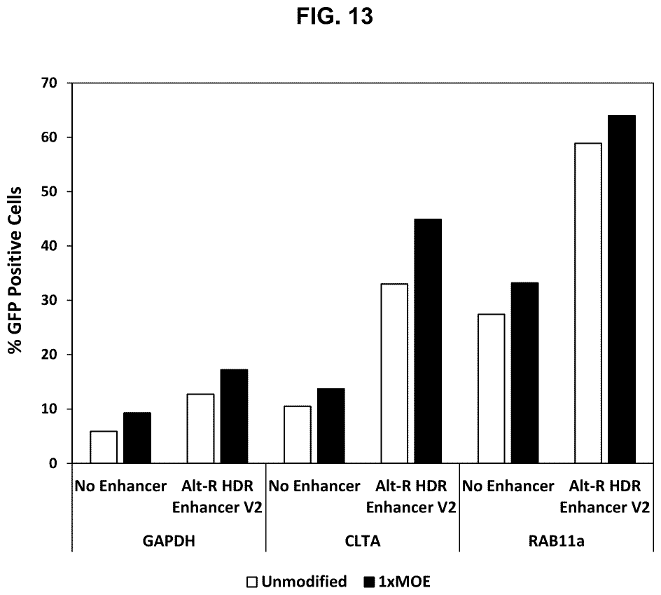

Further analysis of the HDR reads from the 1×MOE modified donors was conducted in the Integrative Genomics Viewer [7] (IGV, Broad Institute, Cambridge, MA). When HDR reads were aligned against either a reference amplicon containing the correct HDR sequence (FIG. 12A) or a reference amplicon containing both the desired insert and the universal priming sequences (FIG. 12B), no evidence of universal sequence incorporation was observed in the HDR reads. Thus, the universal sequences can be incorporated into the manufacturing process of modified dsDNA donors without adversely impacting functional performance.

### Example 9

Use of Modified dsDNA Donors Manufactured With Universal Priming Sequences to Generate GFP Fusions in Human Cell Lines.

To assess the functional performance of modified dsDNA donor templates in applications such as protein tagging, donors were designed to generate GFP tagged GAPDH (C-terminal fusion), CLTA (N-terminal fusion), and RAB11a (N-terminal fusion). Donors were manufactured with universal priming sequences as previously described, using either unmodified or 1×MOE modified primers. Guide and donor sequences used are listed in Table 9. Donors were delivered at 50 nM in a final volume of 28 μL nucleofection buffer with 2 μM Cas9 V3™ RNP (IDT, Coralville, lowa) targeting GAPDH, CLTA, or RAB11a into 3.5×10⁵ K562 cells using Lonza nucleofection (Lonza, Basel, Switzerland). Following the transfection, cells were plated in duplicate wells. For one set of wells, cells were treated with the IDT Alt-R™ HDR Enhancer V2 (1 μM) for 24 hrs post-transfection. Cells were passaged for 7 days, at which point HDR rates were assessed by flow cytometry. Briefly, cells were washed in PBS and then resuspended at 1-2×10⁶ cells/mL. Hoechst 33258 was added to the cell suspension at a final concentration of 4 μg/ml shortly before analysis for viability staining. Cells were analyzed on a Becton Dickinson LSR II cytometer (BD Bioscience, San Jose, CA) to assess GFP expression levels (FIG. 12).

TABLE 9
Sequences of Primers for dsDNA Donor Synthesis
in Example 9

| SEQ | | |
|---|---|---|
| ID | | |
| NO. | Name | Sequence |
| SEQ | GAPDH | AACGACCACTTTGTCAAGCTCATTTCCTGGTATGTGG |
| ID | C-term | CTGGGGCCAGAGACTGGCTCTTAAAAAGTGCAGGGTC |
| NO: | GFP | TGGCGCCCTCTGGTGGCTGGCTCAGAAAAAGGGCCCT |
| 182 | donor | GACAACTCTTTTCATCTTCTAGGTATGACAACGAATT |
| | | TGGCTACAGCAACAGGGTGGTGGACCTCATGGCCCAC |
| | | ATGGCCTCCAAGGAGGGATCTGGCGCCACCAATTTCA |
| | | GCCTGCTGAAACAGGCTGGCGACGTGGAAGAGAACCC |
| | | TGGACCTGTGAGCAAGGGCGAGGAGCTGTTCACCGGG |
| | | GTGGTGCCCATCCTGGTCGAGCTGGACGGCGACGTAA |
| | | ACGGCCACAAGTTCAGCGTGTCCGGCGAGGGCGAGGG |
| | | CGATGCCACCTACGGCAAGCTGACCCTGAAGTTCATC |
| | | TGCACCACCGGCAAGCTGCCCGTGCCCTGGCCCACCC |
| | | TCGTGACCACCCTGACCTACGGCGTGCAGTGCTTCAG |
| | | CCGCTACCCCGACCACATGAAGCAGCACGACTTCTTC |
| | | AAGTCCGCCATGCCCGAAGGCTACGTCCAGGAGCGCA |
| | | CCATCTTCTTCAAGGACGACGGCAACTACAAGACCCG |
| | | CGCCGAGGTGAAGTTCGAGGGCGACACCCTGGTGAAC |
| | | CGCATCGAGCTGAAGGGCATCGACTTCAAGGAGGACG |
| | | GCAACATCCTGGGGCACAAGCTGGAGTACAACTACAA |
| | | CAGCCACAACGTCTATATCATGGCCGACAAGCAGAAG |
| | | AACGGCATCAAGGTGAACTTCAAGATCCGCCACAACA |
| | | TCGAGGACGGCAGCGTGCAGCTCGCCGACCACTACCA |
| | | GCAGAACACCCCCATCGGCGACGGCCCCGTGCTGCTG |
| | | CCCGACAACCACTACCTGAGCACCCAGTCCGCCCTGA |
| | | GCAAAGACCCCAACGAGAAGCGCGATCACATGGTCCT |
| | | GCTGGAGTTCGTGACCGCCGCCGGGATCACTCTCGGC |
| | | ATGGACGAGCTGTACAAGTAAGACCCCTGGACCACCA |
| | | GCCCCAGCAAGAGCACAAGAGGAAGAGAGAGACCCTC |
| | | ACTGCTGGGGAGTCCCTGCCACACTCAGTCCCCCACC |
| | | ACACTGAATCTCCCCTCCTCACAGTTGCCATGTAGAC |
| | | CCCTTGAAGAGGGGAGGGGCCTAGGGAGCCGCACCTT |
| | | GTCATGTACCATCAATAAAGTACCCTGTGCTCA |
| SEQ | CLTA | GTCGTACCGACTGGTAGATGACAGCAAACCTGTTCCC |
| ID | N-term | TTTTCGGCTCTGCAACACCGCCTAGACCGACCGGATA |
| NO: | GFP | CACGGGTAGGGCTTCCGCTTTACCCGTCTCCCTCCTG |
| 183 | donor | GCGCTTGTCCTCCTCTCCCAGTCGGCACCACAGCGGT |
| | | GGCTGCCGGGCGTGGTGTCGGTGGGTCGGTTGGTTTT |
| | | TGTCTCACCGTTGGTGTCCGTGCCGTTCAGTTGCCCG |
| | | CCATGGCTGGATCTGGTGGTACTAGTGGAAGCAAGGG |
| | | TGAGGAGCTGTTCACCGGAGTGGTGCCTATCCTGGTC |
| | | GAGCTGGACGGCGACGTAAACGGTCACAAGTTCAGCG |
| | | TGCGTGGTGAGGGCGAGGGCGATGCCACCAACGGCAA |
| | | GCTGACCCTGAAGTTCATCTGCACCACTGGCAAGCTG |
| | | CCTGTTCCATGGCCAACCCTCGTGACTACACTGACCT |
| | | ACGGCGTTCAGTGCTTCAGCCGTTACCCTGACCATAT |
| | | GAAGCGTCACGACTTCTTCAAGTCTGCCATGCCTGAA |
| | | GGCTACGTCCAGGAGCGTACCATCAGCTTCAAGGACG |
| | | ATGGCACCTACAAGACTCGTGCCGAGGTGAAGTTCGA |
| | | GGGTGACACCCTGGTGAACCGCATCGAGCTGAAGGGT |
| | | ATCGACTTCAAGGAGGACGGCAACATCCTGGGTCACA |
| | | AGCTGGAGTACAACTTCAACAGCCACAACGTCTATAT |
| | | CACCGCCGACAAGCAGAAGAACGGCATCAAGGCCAAC |
| | | TTCAAGATTCGTCACAACGTGGAGGACGGTAGCGTGC |
| | | AGCTCGCAGACCACTACCAGCAGAACACGCCTATCGG |
| | | CGACGGTCCAGTGTTGCTGCCAGACAACCACTACCTG |
| | | AGCACCCAGTCCGTGCTGAGCAAAGACCCGAACGAGA |
| | | AGCGCGATCACATGGTCCTGCTGGAGTTCGTGACCGC |
| | | AGCCGGTATCACTGGAACCGGTGCTGGAAGTGGTGAG |
| | | CTGGATCCGTTCGGCGCCCCTGCCGGCGCCCCTGGCG |
| | | GTCCCGCGCTGGGGAACGGAGTGGCCGGCGCCGGCGA |
| | | AGAAGACCCGGCTGCGGCCTTCTTGGCGCAGCAAGAG |
| | | AGCGAGATTGCGGGCATCGAGAACGACGAGGCCTTCG |
| | | CCATCCTGGACGGCGGCGCCCCCGGGCCCCAGCCGCA |
| | | CGGCGAGCCGCCGATCCGAAAACGGGCGTATAGTCGA |
| | | GACC |
| SEQ | RAB11a | TCAGGGGCGGGGCGCCGCCCCCGGAAGTACTTCCCCT |
| ID | N-term | TAAAGGCTGGGGCCTGCCGGAAATGGCGCAGCGGCAG |
| NO: | GFP | GGAGGGGCTCTTCACCCAGTCCGGCAGTTGAAGCTCG |
| 184 | donor | GCGCTCGGGTTACCCCTGCAGCGACGCCCCCTGGTCC |
| | | CACAGATACCACTGCTGCTCCCGCCCTTTCGCTCCTC |
| | | GGCCGCGCAATGGGCGGATCGGGTGGGACTAGTGGCA |
| | | GCAAGGGCGAGGAGCTGTTCACCGGGGTGGTGCCCAT |
| | | CCTGGTCGAGCTGGACGGCGACGTAAACGGCCACAAG |
| | | TTCAGCGTGCGCGGCGAGGGCGAGGGCGATGCCACCA |
| | | ACGGCAAGCTGACCCTGAAGTTCATCTGCACCACCGG |
| | | CAAGCTGCCCGTGCCCTGGCCCACCCTCGTGACCACC |
| | | CTGACCTACGGCGTGCAGTGCTTCAGCCGCTACCCCG |
| | | ACCACATGAAGCGCCACGACTTCTTCAAGTCCGCCAT |
| | | GCCCGAAGGCTACGTCCAGGAGCGCACCATCAGCTTC |
| | | AAGGACGACGGCACCTACAAGACCCGCGCCGAGGTGA |
| | | AGTTCGAGGGCGACACCCTGGTGAACCGCATCGAGCT |
| | | GAAGGGCATCGACTTCAAGGAGGACGGCAACATCCTG |
| | | GGGCACAAGCTGGAGTACAACTTCAACAGCCACAACG |
| | | TCTATATCACCGCCGACAAGCAGAAGAACGGCATCAA |
| | | GGCCAACTTCAAGATCCGCCACAACGTGGAGGACGGC |
| | | AGCGTGCAGCTCGCCGACCACTACCAGCAGAACACCC |
| | | CCATCGGCGACGGCCCCGTGCTGCTGCCCGACAACCA |
| | | CTACCTGAGCACCCAGTCCGTGCTGAGCAAAGACCCC |
| | | AACGAGAAGCGCGATCACATGGTCCTGCTGGAGTTCG |
| | | TGACCGCCGCCGGGATCACTGGAACCGGTGCTGGAAG |
| | | TGGTACACGCGACGACGAGTACGACTACCTCTTTAAA |
| | | GGTGAGGCCATGGGCTCTCGCACTCTACACAGTCCTC |
| | | GTTCGGGGACCCGGGCCACTCCCGGTGGACCCTCGTG |
| | | CCGGCCACCCCTGCACTGATATAGGCCTCCCTCAGCC |
| | | CTTCCTTTTTGTGCGGTTCCGTCTCCTACCCAGCTCA |
| | | GCCTCTTCTCCCCCGCTCA |
| SEQ | GAPDH | CCTCCAAGGAGTAAGACCCC |
| ID | guide | |
| NO: | proto- | |
| 185 | spacer | |
| SEQ | CLTA | GAACGGATCCAGCTCAGCCA |
| ID | guide | |
| NO: | proto- | |
| 186 | spacer | |
| SEQ | RAB11a | GGTAGTCGTACTCGTCGTCG |
| ID | guide | |
| NO: | proto- | |
| 187 | spacer | |

DNA is uppercase. All primers and templates were synthesized by IDT (Coralville, IA).

Overall HDR rates varied across the sites tested, with maximum GFP positive rates of 17.2% (GAPDH), 44.9% (CLTA), and 64% (RAB11a) achieved under optimal conditions. No GFP signal was observed in cells that received a dsDNA donor without RNP (data not shown). HDR rates were increased with modified dsDNA donor templates in both untreated conditions (1.6, 1.3, and 1.2-fold improvement over unmodified dsDNA for GAPDH, CLTA, and RAB11a respectively) and in HDR Enhancer treated conditions (1.4, 1.4, and 1.1-fold improvements over unmodified dsDNA respectively). On average, use of 1×MOE modified dsDNA donors increased HDR rates 1.3-fold over unmodified dsDNA donors across all conditions. In comparison, use of the Alt-R HDR Enhancer V2 increased HDR rates on average 2.4-fold across all sites and conditions. The combined use of modified donors and HDR Enhancer boosted HDR rates 3.2-fold on average across all sites. Taken together, this demonstrates the combined utility of using optimal reagents (i.e. modified donors and small molecule enhancers) in HDR experiments.

### Example 10

Use of Universal Priming Sequences Enables Greater Consistency and Improved Yields When Manufacturing dsDNA HDR Templates

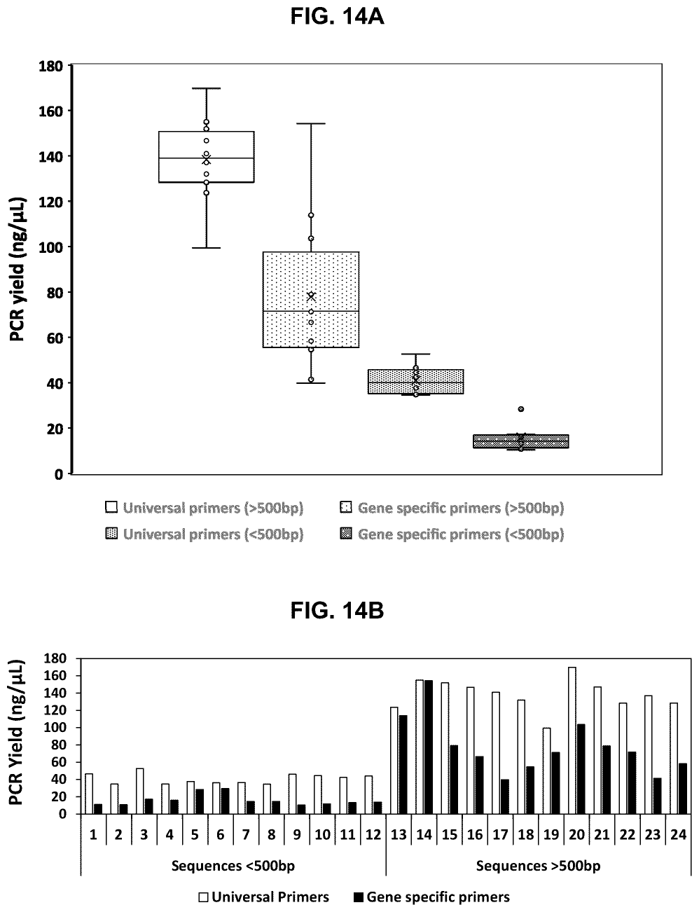

In order to assess the impact of universal priming sequences on the manufacturing process of dsDNA HDR templates, 24 sequences were generated using either universal priming sequences (Table 8, SEQ ID NO: 172-181) or gene specific primers (Table 10, SEQ ID NO: 188-235) with varying modifications. As previously described, all donors were produced by amplification from a plasmid (pUCIDT Amp or pUCIDT Kan vectors) containing the sequence of interest. PCR amplifications were conducted using KOD Hot Start DNA Polymerase (EMD) according to the manufacturer's recommendations, with 200 nM primers and 10 ng plasmid DNA in a 50 μL final reaction volume. Thermocycling was conducted using a Bio-Rad S1000 thermal cycler with the following cycling conditions: a 3 min incubation at 95° C., followed by 36 amplification cycles (95° C. for 20 sec; 65° C. for 10 sec; 70° C. for 20-30 sec/kb). Annealing temperatures were adjusted according to the gene specific primer melting temperatures. Following a SPRI bead cleanup, all products were analyzed using Fragment Analyzer (Agilent) and sequence verified by NGS using the Illumina-Nextera DNA Library Preparation Kit. Overall amplification efficiencies from universal primers or gene specific primers were assessed by measuring final yields, reported as ng/μL (FIG. 14A).

TABLE 10
Sequences of Primers for dsDNA Donor
Synthesis in Example 10.

| | | | Am- |
|---|---|---|---|
| | | | plicon |
| SEQ ID | | | Length |
| NO. | Name | Sequence | (bp) |
| SEQ ID | Gene | ACGAAGTGTTGGATATA | 152 |
| NO: | specific | AGCCAGACTGTAAGTGA | |
| 188 | F1 | | |
| SEQ ID | Gene | TCTAAGCAATTATAAGC | 152 |
| NO: | specific | CATTTCACATAAAACTC | |
| 189 | R1 | TTTTAGGTTAAA | |
| SEQ ID | Gene | MACGAAGTGTTGGATATA | 152 |
| NO: | specific | AGCCAGACTGTAAGTGA | |
| 190 | F2 | | |
| SEQ ID | Gene | MTCTAAGCAATTATAAGC | 152 |
| NO: | specific | CATTTCACATAAAACTCT | |
| 191 | R2 | TTTAGGTTAAA | |
| SEQ ID | Gene | GCCCTGTAGTCTCTCTGT | 198 |
| NO: | specific | ATGTTATATGTCACATTT | |
| 192 | F3 | TGTAA | |
| SEQ ID | Gene | AAGTAATTCACTTACAGT | 198 |
| NO: | specific | CTGGCTTATATCCAACAC | |
| 193 | R3 | TTCG | |
| SEQ ID | Gene | MGCCCTGTAGTCTCTCTG | 198 |
| NO: | specific | TATGTTATATGTCACATT | |
| 194 | F4 | TTGTAA | |
| SEQ ID | Gene | MAAGTAATTCACTTACAG | 198 |
| NO: | specific | TCTGGCTTATATCCAACA | |
| 195 | R4 | CTTCG | |
| SEQ ID | Gene | AGCTTGCTGGTGAAAAGG | 282 |
| NO: | specific | ACCCCA | |
| 196 | F5 | | |
| SEQ ID | Gene | AATGTGCCTCTCTACAAA | 282 |
| NO: | specific | TATTCTCTAAGCAATTAT | |
| 197 | R5 | AAGCCATTTC | |
| SEQ ID | Gene | MAGCTTGCTGGTGAAAAG | 282 |
| NO: | specific | GACCCCA | |
| 198 | F6 | | |
| SEQ ID | Gene | MAATGTGCCTCTCTACAA | 282 |
| NO: | specific | ATATTCTCTAAGCAATTA | |
| 199 | R6 | TAAGCCATTTC | |
| SEQ ID | Gene | ACGTCAGTCTTCTCTTT | 951 |
| NO: | specific | TGTAATGCCCTGTAGTC | |
| 200 | F7 | | |
| SEQ ID | Gene | GATGGTTAAATGATTGAC | 1456 |
| NO: | specific | AAAAAAAGTAATTCACTT | |
| 201 | R7 | ACAGTCTGG | |
| SEQ ID | Gene | MACGTCAGTCTTCTCTTT | 2170 |
| NO: | specific | TGTAATGCCCTGTAGTC | |
| 202 | F8 | | |
| SEQ ID | Gene | MGATGGTTAAATGATTGA | 2170 |
| NO: | specific | CAAAAAAAGTAATTCACT | |
| 203 | R8 | TACAGTCTGG | |
| SEQ ID | Gene | TGTAGTCTCTCTGTATGT | 2170 |
| NO: | specific | TATATGTCACATTTTGTA | |
| 204 | F9 | ATTAACAGCT | |
| SEQ ID | Gene | ATTTAGATAAAGAAAACA | 2170 |
| NO: | specific | TCACTTTTAAATCTAATA | |
| 205 | R9 | CTGGCAAATG | |
| SEQ ID | Gene | MTGTAGTCTCTCTGTATG | 2567 |
| NO: | specific | TTATATGTCACATTTTGT | |
| 206 | F10 | AATTAACAGCT | |
| SEQ ID | Gene | MATTTAGATAAAGAAAAC | 2814 |
| NO: | specific | ATCACTTTTAAATCTAAT | |
| 207 | R10 | ACTGGCAAATG | |
| SEQ ID | Gene | CATGGTACACTCAGCACG | 2955 |
| NO: | specific | GATGAAATGAAACAG | |
| 208 | F11 | | |
| SEQ ID | Gene | AGCAATTATAAGCCATTT | 2955 |
| NO: | specifi | CACATAAAACTCTTTTAG | |
| 209 | R11 | GTTAAAGATG | |
| SEQ ID | Gene | MCATGGTACACTCAGCA | 2955 |
| NO: | specific | CGGATGAAATGAAACAG | |
| 210 | F12 | | |
| SEQ ID | Gene | MAGCAATTATAAGCCATT | 2955 |
| NO: | specific | TCACATAAAACTCTTTTA | |
| 211 | R12 | GGTTAAAGATG | |
| SEQ ID | Gene | MTCTCAGATTCCAGTTTC | 152 |
| NO: | specific | AGCAAATTTGCTTGATAT | |
| 212 | F13 | GTACAGC | |
| SEQ ID | Gene | MTGAATAGAGTGGTTGCA | 152 |
| NO: | specific | CAAACTTACGGATCATTT | |
| 213 | R13 | G | |
| SEQ ID | Gene | MATGGTGAGCAAGGGCGA | 152 |
| NO: | specific | GGAGCT | |
| 214 | F14 | | |
| SEQ ID | Gene | MAGAGTGATCCCGGCGGC | 152 |
| NO: | specific | GGTCA | |
| 215 | R14 | | |
| SEQ ID | Gene | CCCACAATTCGCTCTCAC | 198 |
| NO: | specific | CAAACCTGAG | |
| 216 | F15 | | |
| SEQ ID | Gene | AGTAGTAATAGTAGTAGT | 198 |
| NO: | specific | ATTAAATAATTTGATAAA | |
| 217 | R15 | TAATTTTAGCAATATAGT | |
| | | TTTTTGT | |
| SEQ ID | Gene | MCCCACAATTCGCTCTC | 198 |
| NO: | specific | ACCAAACCTGAG | |
| 218 | F16 | | |
| SEQ ID | Gene | MAGTAGTAATAGTAGTAG | 198 |
| NO: | specific | TATTAAATAATTTGATAA | |
| 219 | R16 | ATAATTTTAGCAATATAG | |
| | | TTTTTTGT | |
| SEQ ID | Gene | MCMCMCACAATTCGCTCT | 282 |
| NO: | specific | CACCAAACCTGAG | |
| 220 | F17 | | |
| SEQ ID | Gene | MAMGMTAGTAATAGTAGT | 282 |
| NO: | specific | AGTATTAAATAATTTGAT | |
| 221 | R17 | AAATAATTTTAGCAATAT | |
| | | AGTTTTTTGT | |
| SEQ ID | Gene | B-C*C*C*A*C*AATTC | 282 |
| NO: | specific | GCTCTCACCAAACCTGAG | |
| 222 | F18 | | |
| SEQ ID | Gene | B-A*G*T*A*G*TAATAG | 282 |
| NO: | specific | TAGTAGTATTAAATAATT | |
| 223 | R18 | TGATAAATAATTTTAGCA | |
| | | ATATAGTTTTTTGT | |
| SEQ ID | Gene | B-G*G*T*A*C*AAGTGG | 951 |
| NO: | specific | ATTTGACTAATTACGAGT | |
| 224 | F19 | GGCTTGATAA | |
| SEQ ID | Gene | B-A*A*A*C*A*ATGCAC | 1456 |
| NO: | specific | TCACTTCTTCCTAGAGAA | |
| 225 | R19 | GAGTACATTC | |
| SEQ ID | Gene | MCCTATTAAATAAAAGAA | 2170 |
| NO: | specific | TAAGCAGTATTATTAAGT | |
| 226 | F20 | AGCCCTGCATTTCA | |
| SEQ ID | Gene | MCATCTGCTTTTTTCCCG | 2170 |
| NO: | specific | TGTCATTCTCTGGACTG | |
| 227 | | R20 | |
| SEQ ID | Gene | CCCACAATTCGCTCTCA | 2170 |
| NO: | specific | CCAAACCTGAG | |
| 228 | F21 | | |
| SEQ ID | Gene | AGTAGTAATAGTAGTAGT | 2170 |
| NO: | specific | ATTAAATAATTTGATAAA | |
| 229 | R21 | TAATTTTAGCAATATAGT | |
| | | TTTTTGT | |
| SEQ ID | Gene | MCCCACAATTCGCTCTC | 2567 |
| NO: | specific | ACCAAACCTGAG | |
| 230 | F22 | | |
| SEQ ID | Gene | MAGTAGTAATAGTAGTA | 2814 |
| NO: | specific | GTATTAAATAATTTGAT | |
| 231 | R22 | AAATAATTTTAGCAATA | |
| | | TAGTTTTTTGT | |
| SEQ ID | Gene | MCMCMCACAATTCGCTC | 2955 |
| NO: | specific | TCACCAAACCTGAG | |
| 232 | F23 | | |
| SEQ ID | Gene | MAMGMTAGTAATAGTAG | 2955 |
| NO: | specific | TAGTATTAAATAATTTG | |
| 233 | R23 | ATAAATAATTTTAGCAA | |
| | | TATAGTTTTTTGT | |
| SEQ ID | Gene | B-C*C*C*A*C*AATTCG | 2955 |
| NO: | specific | CTCTCACCAAACCTGAG | |
| 234 | F24 | | |
| SEQ ID | Gene | B-A*G*T*A*G*TAATAG | 2955 |
| NO: | specific | TAGTAGTATTAAATAATT | |
| 235 | R24 | TGATAAATAATTTTAGCA | |
| | | ATATAGTTTTTTGT | |

DNA is uppercase; 2′-O-methoxyethyl modified ribonucleotides are shown with an uppercase M preceeding the modified ribonucleotide; B- is a 5′-biotin moiety; and phosphorothioate (PS) modified linkages are shown with an asterisk (*). All primers and templates were synthesized by IDT (Coralville, IA).

Owing to differences in the yields for short (<500 bp) and long (>500 bp) amplicons, overall yields following amplification with either universal or gene specific primers were assessed separately for 12 short and 12 long HDR templates (FIG. 14A). Overall, yields were significantly higher with the use of universal primers for both short and long amplicons. For long amplicons, use of universal primers resulted in an average concentration of 138.3 ng/μL (+18.0 SD) following cleanup while use of gene specific primers resulted in an average concentration of 77.8 ng/μL (±32.6 SD). For short amplicons, use of universal primers resulted in an average concentration of 40.9 ng/μL (±5.9 SD) following cleanup while use of gene specific primers resulted in an average concentration of 15.9 ng/μL (±6.4 SD). Direct comparisons between each sequence amplified with universal or gene specific primers (FIG. 14B) reveals large variation in the yields when using gene specific primers. In contrast, use of universal primers results in both higher yields (2.9-and 2.0-fold improvements on average for short and long amplicons, respectively) and greater consistency in the yields across sequences of similar length. Owing to the higher yields and greater consistency, the use of universal primers will better support the development of high-throughput manufacturing processes.

What is claimed is:

1. A programmable nuclease system comprising:
a modified double stranded DNA homology directed repair (HDR) donor comprising:
a first homology arm region,
an insert region,
a second homology arm region; and
universal primer sequences terminally flanking the first homology arm region and the second homology arm region;
wherein:
the first homology arm region and the second homology arm region comprise one or more 2′-OME, 2′-MOE, 2′-F, or 2′-oxygen-4′-carbon methylene (Locked Nucleic Acid) modifications of the 5′-terminal nucleotide, the 5′-penulimate nucleotide, the 5′-antepenultimate (third) nucleotide, or a combination of the nucleotides at or near the 5′-terminus of the first homology arm region and the second homology arm region;
the 2′-OME and 2′-MOE modifications improve homology directed repair efficiency and reduce homology-independent integration; and
the Locked Nucleic Acid and 2′-F modifications improve homology directed repair efficiency and increase homology-independent integration;
a programmable nuclease enzyme, and
a gRNA, wherein the gRNA molecule is capable of targeting the programmable nuclease molecule to a target nucleic acid.

2. The programmable nuclease system of claim 1, wherein the modified double stranded DNA HDR donor comprises one or more 2′-MOE modifications at or near the 5′-termini.

3. The programmable nuclease system of claim 1, wherein the modification at or near the 5′-termini are non-template mismatches relative to a target DNA.

4. The programmable nuclease system of claim 1, wherein the first homology arm region and the second homology arm region are 40 to 150 nucleotides in length.

5. The programmable nuclease system of claim 1, wherein the first homology arm region and the second homology arm region are at least 100 nucleotides in length.

6. The programmable nuclease system of claim 1, wherein the insert region is greater than 100 bp.

7. The programmable nuclease system of claim 1, wherein the insert region is greater than 0.25 kb, greater than 0.5 kb, greater than 1 kb, greater than 2 kb, greater than 3 kb, greater 4 kb, greater than 5 kb, greater than 6 kb, greater than 7 kb, greater than 8 kb, greater than 9 kb, or greater than 10 kb.

8. The programmable nuclease system of claim 1, wherein the programmable nuclease system comprises one or more of transcription activator-like effector nucleases (Taalense), zinc fingers (ZFNs), or clustered, regularly interspaced, short palindromic repeat (CRISPR).

9. The programmable nuclease system of claim 1, wherein the programmable nuclease system is CRISPR.

10. The programmable nuclease system of claim 1, wherein the programmable nuclease enzyme is CRISPR associated-9 (Cas9).

11. The programmable nuclease system of claim 1, wherein the programmable nuclease system further comprises one or more HDR enhancers.

12. A method for increasing homology directed repair (HDR) rates and reducing homology-independent integration in a programmable nuclease system comprising targeting a candidate editing target site locus with an active programmable nuclease system and a modified double stranded DNA HDR donor, wherein the modified double stranded donor comprises:
a first homology arm region,
an insert region,
a second homology arm region; and
universal primer sequences terminally flanking the first homology arm region and the second homology arm region;
wherein:
the first homology arm region and the second homology arm region comprise one or more 2′-OME, 2′-MOE, 2′-F, or 2′-oxygen-4′-carbon methylene (Locked Nucleic Acid) modifications of the 5′-terminal nucleotide, the 5′-penulimate nucleotide, the 5′-antepenultimate (third) nucleotide, or a combination of the nucleotides at or near the 5′-terminus of the first homology arm region and the second homology arm region;
the 2′-OME and 2′-MOE modifications improve homology directed repair efficiency and reduce homology-independent integration; and
the Locked Nucleic Acid and 2′-F modifications improve homology directed repair efficiency and increase homology-independent integration

13. The method of claim 9, wherein the modified double stranded DNA HDR donor comprises one or more 2′-MOE modifications at or near the 5′-termini.

14. The method of claim 9, wherein the method further comprises one or more HDR enhancers.

15. The method of claim 9, wherein the first homology arm region and the second homology arm region are at least 100 nucleotides in length.

16. The method of claim 9, wherein the insert region is greater than 100 bp.

17. A method for manufacturing a modified double stranded DNA HDR donor, the method comprising synthesizing or amplifying a first oligonucleotide comprising a first homology arm region, an insert region, a second homology arm region; and a second oligonucleotide that is complementary to the first oligonucleotide; and hybridizing the first and second oligonucleotide; wherein the first homology arm region and the second homology arm region comprise modifications to one or more nucleotides at or near the 5′-termini; wherein the modified double stranded DNA HDR donor comprises:
the first homology arm region,
the insert region,
the second homology arm region; and
universal primer sequences terminally flanking the first homology arm region and the second homology arm region;
wherein:
the first homology arm region and the second homology arm region comprise one or more 2′-OME, 2′-MOE, 2′-F, or 2′-oxygen-4′-carbon methylene (Locked Nucleic Acid) modifications of the 5′-terminal nucleotide, the 5′-penulimate nucleotide, the 5′-antepenultimate (third) nucleotide, or a combination of the nucleotides at or near the 5′-terminus of the first homology arm region and the second homology arm region;
the 2′-OME and 2′-MOE modifications improve homology directed repair efficiency and reduce homology-independent integration; and
the Locked Nucleic Acid and 2′-F modifications improve homology directed repair efficiency and increase homology-independent integration.

18. The method of claim 17, wherein the modified double stranded DNA HDR donor comprises one or more 2′-MOE modifications at or near the 5′-termini.

19. The method of claim 17, wherein the first homology arm region and the second homology arm region are at least 100 nucleotides in length.

20. The method of claim 17, wherein the insert region is greater than 100 bp.
